SEQUENCE LISTING (12 sequences)

SEQ ID NO: 1 - DNA, 24 bp, synthetic construct
  source 1..24: mol_type = other DNA
gaaatcccatcaccatcttccagg

SEQ ID NO: 2 - DNA, 20 bp, synthetic construct
  source 1..20: mol_type = other DNA
gagccccagccttctccatg

SEQ ID NO: 3 - DNA, 20 bp, synthetic construct
  source 1..20: mol_type = other DNA
gggagggttgattgcctaaa

SEQ ID NO: 4 - DNA, 23 bp, synthetic construct
  source 1..23: mol_type = other DNA
gacaagttgcctgtgtgtttatt

SEQ ID NO: 5 - DNA, 24 bp, synthetic construct
  source 1..24: mol_type = other DNA
tgtcctggatgttgactgccttga

SEQ ID NO: 6 - DNA, 24 bp, synthetic construct
  source 1..24: mol_type = other DNA
tgtcgcaccactgataccctgaaa

SEQ ID NO: 7 - DNA, 21 bp, synthetic construct
  source 1..21: mol_type = other DNA
gatccaggcaaatctgcaata

SEQ ID NO: 8 - DNA, 20 bp, synthetic construct
  source 1..20: mol_type = other DNA
tggtctaaccagggtctctt

SEQ ID NO: 9 - DNA, 24 bp, synthetic construct
  source 1..24: mol_type = other DNA
gacaagaggagaagcagagattag

SEQ ID NO: 10 - DNA, 19 bp, synthetic construct
  source 1..19: mol_type = other DNA
tgaggtattagtcgcattt

SEQ ID NO: 11 - DNA, 23 bp, synthetic construct
  source 1..23: mol_type = other DNA
gaagaggagatagtggagcaaac

SEQ ID NO: 12 - DNA, 22 bp, synthetic construct
  source 1..22: mol_type = other DNA
gctgacgatgtcccatgatatt

(19) United States
(12) Patent Application Publication
(10) Pub. No.: US 2026/0335323 A1
(43) Pub. Date: Oct. 29, 2026

(54) CHIMERIC MOLECULE

(71) Applicant: UCL Business Ltd, London (GB)

(72) Inventors: Robin Ketteler, London (DE); Niyaz Zaman, London (GB); David Selwood, London (GB)

(21) Appl. No.: 19/485,520

(22) PCT Filed: May 17, 2024
(86) PCT No.: PCT/GB2024/051303

§ 371 Date: Nov. 18, 2025

(30) Foreign Application Priority Data
May 18, 2023 (GB) 2307491.7
May 23, 2023 (GB) 2307725.8

(51) Int. Cl. A61K 31/519; A61K 31/167; A61K 47/54; A61K 47/55; C07D 495/14

(52) U.S. Cl. CPC A61K 31/519; A61K 31/167; A61K 47/545; A61K 47/55; C07D 495/14

(57) ABSTRACT

The present invention provides a chimeric molecule for inducing degradation of a target in a cell, the chimeric molecule comprising an autophagy-inducing moiety and a targeting moiety.

### FIELD OF THE INVENTION

The present invention relates to a chimeric molecule for inducing degradation of a target in a cell, the chimeric molecule comprising an autophagy-inducing moiety and a targeting moiety.

### BACKGROUND TO THE INVENTION

Autophagy is a cellular process that eliminates molecules and other sub-cellular entities such as organelles through lysosome-mediated degradation. In autophagy, a double-membraned organelle called an autophagosome forms and elongates around matter to be degraded, such as dysfunctional organelles and protein aggregates. After encapsulating the target, the autophagosome fuses with a lysosome, and the hydrolytic enzymes therein degrade the target. Autophagy of mitochondria is referred to as mitophagy. Autophagy of peroxisomes is referred to as pexophagy. The cellular molecular machinery of autophagy is described in review articles such as Dikic and Elazar Nat Rev Mol Cell Biol 19, 349-364 (2018) and Aman et al. Nature Aging, Vol 1, August 2021, 634-650.

Targeted manipulation of autophagy offers opportunities for new discoveries into the regulation of cell biological processes and the development of therapeutic strategies for indications such as cancer and neurodegenerative diseases. Genetic interference with key autophagy genes has proven very useful to dissect the molecular basis of autophagy, identify potential drug targets and understand the role of autophagy in development and disease. However, genetic modifications are technically challenging and time-consuming. On the other hand, chemical approaches involving small molecule inhibitors and activators operate on a much shorter time scale and modulate autophagy in an acute, reversible manner. Compounds have been developed to target a selected number of a few key proteins in the pathway such as, p62/SQSTM1 inhibitors4, ULK1/2 inhibitors and activators and ATG4B inhibitors. There are several limitations in small molecule discovery, notably the inability to target what is considered the non-druggable proteome and an inability to direct the modulation locally. Strategies for targeting other mechanisms such as protein-protein interactions or transcription has been successful in some instances, yet the field is still in its infancy.

c-MYC is a transcription factor involved in a range of growth and metabolic signalling. c-MYC is aberrantly expressed in over 70% of human cancers, where it drives proliferation of cancer cells.

BRD4 is a soluble protein involved in enhancing c-MYC-dependent transcription, including the transcription of c-MYC itself.

Neurodegeneration refers to a progressive structural and functional loss of neurons causing heterogeneous clinical and pathological expressions followed by deterioration of functional anatomy. This progressive neuronal cell death often leads to various neurodegenerative disorders (NDDs) such as Parkinson's disease (PD), Huntington's disease (HD), Alzheimer's disease (AD), amyotrophic lateral sclerosis (ALS), brain trauma (BT), prion disease (PrD), progressive supranuclear palsy (PSP), and spinocerebellar ataxias (SCA). It includes associated neuropathology, disease based anatomical vulnerability, and aggregation of some major selective proteins during disease conditions. In the last few decades, several approaches have been taken to understand the mechanisms of neuronal cell death. The oxidative and nitrosative stress due to the overproduction of reactive oxygen species (ROS) and reactive nitrogen species (RNS) with the deterioration of cellular antioxidant defense systems are found to be the major reasons behind this neuronal cell damage which might further lead to NDDs.

The treatment of these neurodegenerative diseases are mostly symptomatic such as dopaminergic treatment for PD and movement disorders, anti-inflammatory and analgesic for neuronal infections and pain, cholinesterase for cognitive disorders, antipsychotic for dementia, etc. though, further progress in therapeutic management is needed to treat many other progressive and serious symptoms of the diseases. Integrative treatments along with medicinal therapies are also in the frontline of research to improve the endogenous antioxidant systems targeting the oxidoreductase enzymes and thereby the activity of daily life of the neurodegenerative patients. These integrated treatments act by protecting against oxidative and nitrosative stress related neuropsychiatric disorders, sensory and other symptoms of non-motor fluctuations, fatigue, etc.

There remains a need for further approaches for therapeutic interventions in fields, such as cancer and neurodegenerative diseases—in particular, where disease aetiology may be associated with aberrant protein expression or an accumulation of dysfunctional cellular material (e.g. proteins and/or organelles).

### SUMMARY OF THE INVENTION

The present invention provides a chimeric molecule for inducing degradation of a target in a cell, the chimeric molecule comprising an autophagy-inducing moiety and a targeting moiety.

The overall function of the chimeric molecule of the invention is to direct the endogenous autophagy machinery of the cell towards a deleterious target, such as an aberrantly expressed protein, a protein aggregate or a dysfunctional organelle, to thereby treat the cause of disease. The targeting moiety associates with the target whilst the autophagy-inducing moiety initiates formation of an autophagosome, which engulfs the target leading to its degradation. Thus, the chimeric molecule co-locates the autophagic machinery with the target to be degraded.

Particular examples described herein are chimeric molecules comprising either c-MYC-targeting moiety or a BRD4-targeting moiety, along with an autophagy-inducing moiety that is an ULK1 agonist. Such molecules direct autophagy towards c-MYC or the c-MYC-enhancing molecule BRD4, which may be useful in the treatment of cancer, particularly cancers that aberrantly express c-MYC.

Also described herein are chimeric molecules comprising a mitochondria-targeting moiety and an autophagy-inducing moiety that is an ULK1 agonist. Such molecules direct autophagy towards dysfunctional mitochondria (i.e. the molecules induce mitophagy), which may be useful in the treatment of Parkinson's Disease.

The chimeric molecules of the present invention are particularly advantageous in that they induce autophagy at the site of the target. In particular, the autophagy-inducing moiety initiates autophagosome formation (i.e. nucleation and extension of the autophagosome membrane) at the target. In contrast, known molecules for targeting autophagy to targets are reliant on the presence of an existing autophagosome and the ability to traffic the target to the autophagosome. The chimeric molecules of the present invention overcome these limitations.

A further advantage is the increased potency of the chimeric molecules of the invention compared to other mitochondrial degraders such as AUTAC 4 (as shown in Example 1 herein). Another advantage is that cellular homeostasis is not disrupted as existing autophagosomes which are important for maintaining cellular homeostasis are not used by the invention; instead, new, targeted autophagosomes are created.

In one embodiment, the (BRD4-targeting) chimeric molecule has the following structure:

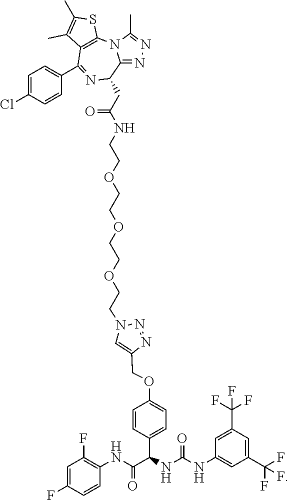 [SMILES: Cc1sc2c(c1C)C(c1ccc(Cl)cc1)=N[C@@H](CC(=O)NCCOCCOCCOCCn1cc(COc3ccc([C@@H](NC(=O)Nc4cc(C(F)(F)F)cc(C(F)(F)F)c4)C(=O)Nc4ccc(F)cc4F)cc3)nn1)c1nnc(C)n1-2]

In one embodiment, the (c-MYC-targeting) chimeric molecule has the following structure:

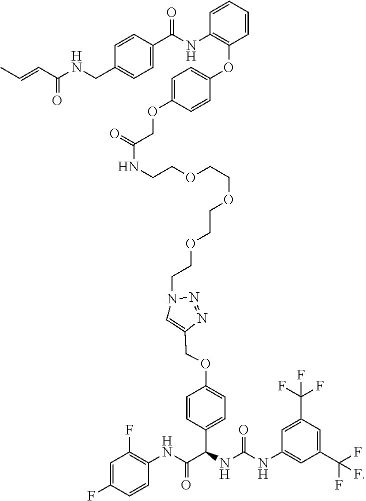 [SMILES: C/C=C/C(=O)NCc1ccc(C(=O)Nc2ccccc2Oc2ccc(OCC(=O)NCCOCCOCCOCCn3cc(COc4ccc([C@@H](NC(=O)Nc5cc(C(F)(F)F)cc(C(F)(F)F)c5)C(=O)Nc5ccc(F)cc5F)cc4)nn3)cc2)cc1]

In one embodiment, the (mitochondria-targeting) chimeric molecule has the following structure:

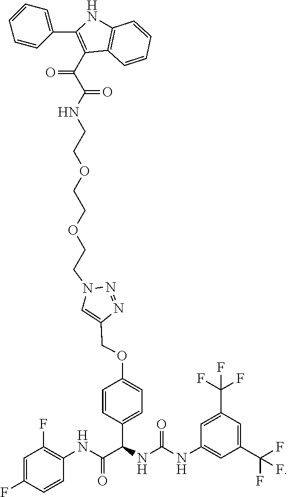 [SMILES: O=C(Nc1cc(C(F)(F)F)cc(C(F)(F)F)c1)N[C@@H](C(=O)Nc1ccc(F)cc1F)c1ccc(OCc2cn(CCOCCOCCNC(=O)C(=O)c3c(-c4ccccc4)[nH]c4ccccc34)nn2)cc1]

In another embodiment, the (mitochondria-targeting) chimeric molecule has the following structure:

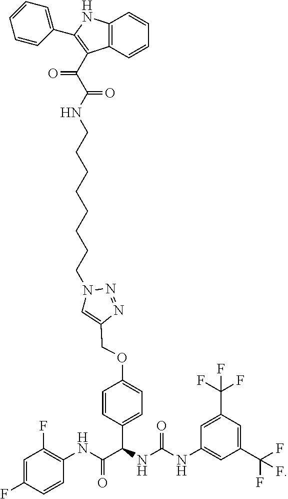 [SMILES: O=C(Nc1cc(C(F)(F)F)cc(C(F)(F)F)c1)N[C@@H](C(=O)Nc1ccc(F)cc1F)c1ccc(OCc2cn(CCCCCCCCNC(=O)C(=O)c3c(-c4ccccc4)[nH]c4ccccc34)nn2)cc1]

The invention also provides a pharmaceutical composition comprising the chimeric molecule according to the invention and one or more of a pharmaceutically acceptable carrier, diluent or excipient.

The invention further provides a method of treating or preventing a disease, the method comprising administering to a subject the chimeric molecule or pharmaceutical composition according to the invention.

The invention also provides the chimeric molecule or the pharmaceutical composition according to the invention for use in treating or preventing a disease in a subject.

The invention also provides a use of the chimeric molecule or the pharmaceutical composition according to the invention in the manufacture of a medicament for treating or preventing a disease in a subject.

The invention further provides an in vitro or in vivo method of inducing degradation of a target in a cell, the method comprising introducing into the cell the chimeric molecule according to the invention.

### DESCRIPTION OF THE FIGURES

FIG. 1. The structure of AUTAC-4 is shown in (A). (B) Representative images of SH-SY5Y cells stably expressing the mito-mKeima reporter treated with AUTAC4 for 18 h. Nuclei were stained with Hoechst 33342, healthy mitochondria are shown in green and mitochondria in lysosomes are shown in red. (C-E) Fold change in mitophagy index over time after treatment with increasing concentrations of AUTAC4 under treatment with DMSO (C), 3 mM CCCP (D) or 0.1/0.1 mM antimycin/oligomycin (E), also for 18 h. (F) Quantification of changes in mitochondrial membrane potential over time (DYₘ) by measuring the TMRM relative fluorescence intensity (RFI) treated with DMSO, 10 mM CCCP, 10 mM and 20 mM AUTAC4. Normalised to number of nuclei. (G) Representative western blot of SH-SY5Y cells treated with DMSO, 10 mM AUTAC4 or 10 mM AUTAC4+0.1/0.1 mM antimycin/oligomycin for 18 h and, probed for p62, b-actin and Tom20. Quantifications of western blot for changes in Tom20 and p62 levels shown in (H) and (I), respectively. (J) Representative images of PANC-1 cells stably expressing the mCherry-EGFP-LC3 tandem reporter. Nuclei stained with Hoechst 33342, autophagosomes are shown in the EGFP channel and autolysosomes in the mCherry channel. Fold change in the number of autophagosome (K) and autolysosome (L) puncta in AUTAC4 treated cells. All data shown are mean±SEM, n=3, *p<0.05, *** p<0.001. For mito-mKeima experiments, statistics were calculated by Two-way ANOVA with Tukey's multiple comparisons test. For western blots, TMRM and LC3 tandem reporter assay, statistics were calculated with one-way ANOVA with Tukey's multiple comparisons test.

FIG. 2. BL-918 is a potent ULK1 agonist and autophagy inducer (A) Representative images of PANC-1 cells stably expressing the mCherry-EGFP-LC3 tandem reporter treated with ULK1 agonists. Nuclei stained with Hoechst 33342, autophagosomes are shown in the EGFP channel and autolysosomes in the mCherry channel. Fold change in the number of autophagosomes (B) and autolysosomes (C) puncta in ULK1 agonist treated cells. (D) Western blot of PANC-1 cells treated with DMSO, bafilomycin and ULK1 agonists±bafilomycin; probed for p-ULK1 (S317), ULK1, p62, b-actin, LC3-I and LC3-II. Quantifications of western blot for changes in the ratio of p-ULK1 (S317):ULK1 (E), p62 (F) and LC31:LC3II (G). All data shown are mean±SEM, n=3, *p<0.05, ** p<0.01, one-way ANOVA with Dunnett's multiple comparisons test.

FIG. 3. (A) Synthetic route for the synthesis of ULKTACs, NZ-65 and NZ-66, see supporting information for complete synthetic procedure: (A) Oxalyl chloride, Et₂O, RT, 90% (B) Azido-PEG2-Amine or 6-Azido-hexylamine, NaHCO₃, DCM, RT, 1=57% and 2=67%, (C) i. N-Methylmorpholine, isobutyl chloroformate, THF, 2,4-difluoroaniline, −20° C. to RT.; ii. 4M HCl in 1,4-dioxane=1/1, RT, 16% (2 steps); (D) i. 3,5-bistrifluoromethylphenyl isocyanate, DIPEA, MeCN, 50° C.; ii. 2M NaOH/THF=1/1,RT. 17% (2 steps) (E) Propargyl bromide, K₂CO₃, MeCN, 80° C.; (F) Compound 1 or 2, Compound 3, THF:H₂O=9:1, CuSO₄, Sodium Ascorbate, 50° C., NZ-65=14%, 2 steps, NZ-66=16%, 2 steps. (B). 3D docking of ULKTACs to ULK1 (Left) and TSPO (Right), poses based upon the kinase domain structure of ULK1 (PDB code: 4WNO) and TSPO (PDB Code: 2MGY). (C) Two-dimensional representation of the modelled 3D structures of ULKTACs binding to ULK1 and TSPO proteins, analysed with BIOVIA Discovery Studio. Interactions are shown by dashed lines: Blue=Halogen Interaction/H-bond, Green=Conventional H-bond, Red=C—H bond, Pink=π-alkyl, Magenta=π-π T-shaped or π-Amide stacked, Orange=π-cation

FIG. 4. ULKTACs retain ULK1 agonist activity and subsequent induction of autophagy (A) Western blot of PANC-1 cells treated with ULKTACs±bafilomycin, probed for p-ULK1 (S317), ULK1, p62, b-Actin, LC31 and LC3II. Quantifications of (A) for the pULK1:ULK1 ratio (B) LC3I:LC3II ratio (C) and changes in p62 (D). (E) Representative images of PANC-1 cells stably expressing the mCherry-EGFP-LC3 tandem reporter treated with ULKTACs w/or w/o bafilomycin. Nuclei stained with Hoechst 33342, autophagosomes are shown in the EGFP channel and autolysosomes in the mCherry channel. Fold change in the number of autophagosome (F) and autolysosome (G) puncta. Data shown are mean±SEM, *p<0.05, ** p<0.01, *** p<0.001, one-way ANOVA, Dunnett's multiple comparisons test.

FIG. 5. ULKTACs induce potent mitophagy after colocalisation and local activation of ULK1 at mitochondria (A) Representative immunofluorescence of PANC-1 cells treated with CCCP, ULKTACs or both and immunostained for TOM20 and ULK1 and counterstained with Alexa 488 and 568, respectively. Nuclei stained with Hoechst 33342. (B) Quantification of the colocalised ULK1 to TOM20 spot area of PANC-1 cells treated with DMSO, 5 mM CCCP, 10 mM BL-918, 10 mM BL-918+5 mM CCCP, 1 mM NZ-65, 1 mM NZ-65+5 mM CCCP, 1 mM NZ-66, 1 mM NZ-66+5 mM CCCP, 50 mM TSPO ligand, 1 mM NZ-65+50 mM TSPO ligand, 1 mM NZ-66+50 mM TSPO ligand. (C) Western blot of SH-SY5Y cells treated with 1 mM ULKTACs, 5 mM CCCP or both, probed for Mfn2, b-Actin and Tom20—quantifications are shown in (D) for Mfn2, normalised to Actin. (E) Representative images of SH-SY5Y cells stably expressing the mito-mKeima reporter treated with 3 mM CCCP, NZ-65 or both. Nuclei were stained with Hoechst 33342, healthy mitochondria are shown in green and mitochondria in lysosomes are shown in red. Fold change in mitophagy index over time after treatment for the indicated timepoints with title treatments alone or co-treated with 1.25 mM ULKTAC or 10 mM ULKTAC NZ-65 (F) or NZ-66 (G). Fold change in mitophagy index over time after treatment for the indicated timepoints with DMSO, 1.25 UM NZ-65 or NZ-66±50 UM TSPO ligand under co-treatment with DMSO (H) or 3 μM CCCP (I). (J) Fold change in TMRM relative fluorescent intensity (and hence DYₘ) after 1 h treatment with DMSO, 3 mM CCCP or 20 mM ULKTAC. Normalised to number of nuclei. All data shown are mean±SEM, n=3, *p<0.05, ** p<0.01, *** p<0.001. For colocalisation and TMRM experiments, one-way ANOVA with Dunnett's multiple comparisons test. For western blots, one-way ANOVA with Tukey's multiple comparisons test and unpaired t-test. For mito-mKeima experiment, two-way ANOVA with Tukey's multiple comparisons test.

FIG. 6. ULKTACs rely on ULK1 for the redirection of autophagy components to mitochondria and subsequent mitophagic degradation (A) Western blot of mouse embryonic fibroblasts (MEF) WT and MEF ULK1/2^{-/-} treated with 1 mM ULKTACs, 5 mM CCCP or both and probed for ULK1, Mfn2 and Actin—quantification of the relative intensity of Mfn2 shown in (B), normalised to Actin. Representative immunofluorescence of MEF WT (C) and MEF ULK1/2^{-/-} (D) treated with 5 mM CCCP, 1 mM ULKTACs or both and immunostained for Alexa 488 ATP5a and Alexa 647 LC3B. Quantification of the colocalised LC3 to ATP5a spot area shown in WT vs ULK1/2^{-/-} treated MEF cells shown in (E). Data shown are mean±SEM, n=3, *p<0.05, ** p<0.01, *** p<0.001, Two-way ANOVA with Tukey's multiple comparisons test.

FIG. 7. ULKTACs restore mitophagic activity in PINK-null PD patient fibroblasts (A) Western blot of patient derived fibroblasts, healthy control vs. PINK null, treated with 1 mM NZ-65 or NZ-66 for 18 h or 10 μM FCCP for 2 h and probed for total Ub, pUb(S65), Mfn2 and b-Actin with quantification for the changes in the pUB (S65): Total Ub ratio shown in (B) and Mfn2 (C), respectively normalised to b-Actin. (D) Representative immunofluorescence of primary human fibroblasts isolated from wild type controls and PINK-null Parkinson's disease patients, treated with 10 UM FCCP, 1 μM NZ-65 or 1 μM NZ-66 and immunostained for TOM20 and ULK1; counterstained with Alexa 488 and Alexa 568 antibodies. Nuclei stained with Hoechst 33342. The quantification of these immunofluorescence experiments is shown in (E). Data shown are mean±SEM, n=3, *** p<0.001, two-way ANOVA with Tukey's multiple comparisons test.

FIG. 8. (A) Diagram of synthesis of NZ-147 by click chemistry. (B) Western blot of CAL51 cells treated with DMSO, (+)-JQ1 or NZ-147. Quantification of BRD4 (C), c-MYC (D) and MAX (E).

FIG. 9. (A) Schematic representation of hTERT-cMYC-ER expressing RPE1 cells. Treatment with 4-hydroxytamoxifen (4-OHT) translocates c-MYC-ER from the cytoplasm into the nucleus, simulating oncogenic c-MYC. (B) Immunofluorescence images of hTERT-cMYC-ER expressing RPE1 cells treated with DMSO, (+)-JQ1 or NZ-147 at 100 nM for 72 hours, each with or without 4-OHT.

FIG. 10. (A) Colony formation assay-images of A549 or H1299 cells treated with DMSO, (+)-JQ1 or NZ-147 at 100 nM for 72 hours and stained with crystal violet. (B) Cell viability assay-percentage of live A549 (black) or H1299 (red) cells after treatment with the indicated concentration of (+)-JQ1 or NZ-147.

FIG. 11. RT-qPCR quantification of expression of c-MYC controlled genes in CAL51 triple negative breast cancer cell line treated with DMSO, 100 nM (+)-JQ1 or 100 nM NZ-147 for 72 hours. (+)-JQ1 reduces the expression of c-MYC controlled genes; NZ-147 has a significantly more profound impact on these targets vs (+)-JQ1 at equivalent concentration and duration of treatment. MDM2 (A) and CCNE (B) are controlled by cMYC in oncogenic settings, PAICS (C) is a putative target of c-MYC, and eIF4E (D) is a known target of cMYC. n=3, *p<0.05, ** p<0.01, *** p<0.001.

FIG. 12. c-MYC and MAX degradation enabled by NZ-175. (A) Schematic representation of hTERT-cMYC-ER expressing RPE1 cells. Treatment with 4-hydroxytamoxifen (4-OHT) translocates c-MYC-ER from the cytoplasm into the nucleus, simulating oncogenic c-MYC. (B) Western blot of RPE1-hTERT-cMYC-ER treated with 4-OHT and/or 100 nM NZ-175 for 72 hours. Quantification of c-MYC-ER (C), c-MYC (D) and MAX (E). NZ-175 initiates degradation of c-MYC, and proximal degradation of MAX. n=3, mean±SEM, *p<0.05, ** p<0.01, *** p<0.001.

FIG. 13. Time-course immunofluorescence of of NZ-175 mediated c-MYC degradation in RPE1-hTERT-cMYC-ER treated with 4-OHT and/or 100 nM NZ-175 for 72 hours.

FIG. 14. RT-qPCR of c-MYC controlled genes in RPE1-hTERT-cMYC-ER cells after treatment with 4-OHT and/or NZ-175 at 100 nM for 72 hours. MDM2 (A) and CCNE (B) are controlled by cMYC in oncogenic settings, PAICS (C) is a putative target of c-MYC, elF4E (D) is a known target of cMYC, and MrdB (E) and ECA39 (F) are putative targets of c-MYC. n=3, mean±SEM, *p<0.05, **<0.01, *** p<0.001.

FIG. 15. NZ-175 rescues oncogenic c-MYC induced replication stress (100 nM, 72 h) (A) Western blot of RPE1-hTERT-cMYC-ER treated with 4-OHT and/or NZ-175 at 100 nM for 72 hours. 4-OHT treatment simulates oncogenic c-MYC-induced replication stress, observed by upregulation of phospho-CHK1 (S345), pRPA (S4/S8) and γH2AX. Degradation of c-MYC with 100 nM NZ-175 for 72 h rescues this phenotype. Quantification of blot is shown for pCHK1 (S317) (B), pRPA (S4/S8) (C), and YH2AX (D). n=3, mean±SEM, *p<0.05, ** p<0.01, *** p<0.001.

FIG. 16. (A) Colony formation assay-images of A549 or H1299 cells treated with DMSO or NZ-175 and stained with crystal violet. (B) Cell viability assay-percentage of live A549 (black) or H1299 (red) cells after treatment with the indicated concentration of NZ-175.

FIG. 17. Chromatogram for NZ-175.

FIG. 18. NMR spectrum for NZ-175.

### DETAILED DESCRIPTION

The invention provides a chimeric molecule for inducing degradation of a target in a cell, the chimeric molecule comprising an autophagy-inducing moiety and a targeting moiety.

In some embodiments, the chimeric molecule further comprises a linker between the autophagy-inducing moiety and the targeting moiety. The linker, when present, connects the autophagy-inducing moiety to the targeting moiety.

In some embodiments, the chimeric molecule is represented by the formula:

A-B—C
wherein
A is the autophagy-inducing moiety,
B is the optional linker, and
C is the targeting moiety.

In some embodiments, the chimeric molecule is represented by the formula:

A-B—C
wherein
A is the autophagy-inducing moiety,
B is the linker, and
C is the targeting moiety.

In some embodiments, the chimeric molecule is represented by the formula:

C—B-A
wherein
A is the autophagy-inducing moiety,
B is the optional linker, and
C is the targeting moiety.

In some embodiments, the chimeric molecule is represented by the formula:

C—B-A
wherein
A is the autophagy-inducing moiety,
B is the linker, and
C is the targeting moiety.

### Chimeric Molecule

The term “chimeric molecule” refers to the molecule of the invention being a combination or “chimera” of two molecules. In other words, the chimeric molecule of the invention comprises two connected constituent molecules, which are referred to herein as “moieties” (i.e. an autophagy-inducing moiety and a targeting moiety).

It will be understood that each “moiety” of the chimeric molecule is itself a “molecule”, but the term “moiety” is used to avoid confusion with the overall “chimeric molecule”. The term “moiety” may therefore be synonymous with “molecule” and “compound”.

Furthermore, the term “moiety” refers to the part (i.e. constituent molecule) of the chimeric molecule that has a particular function (i.e. induction of autophagy or targeting). The autophagy-inducing moiety and the targeting moiety would each independently fulfil their respective function when separate from one another (i.e. when not part of the chimeric molecule of the invention). Thus, the autophagy-inducing moiety induces autophagy when separate from or when part of the chimeric molecule of the invention. Likewise, the targeting moiety associates with a particular target when separate from or when part of the chimeric molecule of the invention.

### Target

As described herein, the chimeric molecule of the invention functions by inducing autophagosome formation at a target. The autophagosome engulfs the target, leading to degradation of the target.

The target is any entity in the cell that may be engulfed by, and degraded within, an autophagosome. In some embodiments, the target is selected from mitochondria, a soluble protein, a transcription factor, a protein aggregate, peroxisomes, a virus, a protein complex, a lipid droplet and a bacterium. Preferably, the target is BRD4. Preferably, the target is c-MYC. Preferably, the target is mitochondria.

### Targeting Moiety

The term “targeting moiety” refers to the part of the chimeric molecule that targets the chimeric molecule to the target. In other words, the targeting moiety associates with the target, thereby locating the chimeric molecule (and thus the autophagy-inducing function of the autophagy-inducing moiety) at the target, causing induction of autophagy at the location of the target. As an isolated molecule/compound (i.e. when not part of the chimeric molecule of the invention), the targeting moiety can independently associate with the target.

The term “associates with” means that the targeting moiety interacts with the target in a manner that causes it to co-locate with the target. Typically, the targeting moiety binds to the target. Thus, in some embodiments, the targeting moiety binds to the target. In some embodiments, the targeting moiety binds to a part or component of the target. In some embodiments, the targeting moiety is a ligand for the target. “Targeting moiety” is synonymous with “targeting ligand”.

In some embodiments, the targeting moiety associates with a protein on the target. In some embodiments, the targeting moiety binds a protein on target.

As described herein, the target may be mitochondria, a soluble protein, a transcription factor, a protein aggregate, peroxisomes, a virus, a protein complex, a lipid droplet and a bacterium. Accordingly, in some embodiments the targeting moiety is selected from a mitochondria-targeting moiety, a protein-targeting moiety, a transcription factor-targeting moiety, a protein aggregate-targeting moiety, a peroxisome-targeting moiety, a virus-targeting moiety, a protein complex-targeting moiety, a lipid droplet-targeting moiety and a bacterium-targeting moiety.

### Mitochondria-Targeting Moieties

In some embodiments the targeting moiety is a mitochondria-targeting moiety.

In some embodiments, the mitochondria-targeting moiety binds a protein on the outer membrane of the mitochondria. Mitochondrial outer membrane proteins are known in the art. In some embodiments, the mitochondria-targeting moiety is an outer mitochondrial membrane protein ligand. In some embodiments, the mitochondria-targeting moiety is selected from a translocation protein (TSPO) ligand, a translocon of the OM (TOM) ligand, a SAM complex ligand, a voltage-dependent anion ion channel ligand and a Mitochondrial import complex (MIM) ligand.

Chimeric molecules comprising a mitochondria-targeting moiety may be particularly useful in the treatment of Parkinson's disease, cardiovascular disease, cancer (both solid tumours and haematological cancer), neurodegeneration with brain iron accumulation (NBIA), diabetes and obesity. In particularly, over-driving mitophagy can be lethal to highly mitophagically-active cancers such as chem-resistant and radio-resistant cancers, cancer stem cells and metastatic cells.

TSPO is a protein on the outer membrane of mitochondria, which is also known as Peripheral Benzodiazepine Receptor (PBR). In some embodiments, the mitochondria-targeting moiety binds TSPO (i.e. the mitochondria-targeting moiety may be a TSPO-binding moiety, TSPO binder or TSPO ligand).

In some embodiments, the TSPO-binding moiety is selected from the TSPO ligands shown in Table 1 below:

TABLE 1

| | |
|---|---|
| 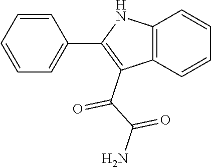 [SMILES: NC(=O)C(=O)c1c(-c2ccccc2)[nH]c2ccccc12] | |
| 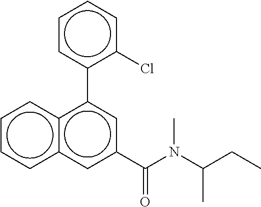 | PK11195 |
| 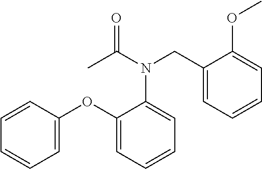 [SMILES: COc1ccccc1CN(C(C)=O)c1ccccc1Oc1ccccc1] | PRB28 |

Other TSPO ligands are described in Zhang et al. 2021 (Zhang et al. Acta Pharmaceutica Sinica B, Vol 11, Issue 2, February 2021, pages 373-393), and any of these may be used as the targeting moiety in the chimeric molecule of the invention.

### Soluble Protein-Targeting Moieties

In some embodiments the targeting moiety is a soluble protein-targeting moiety. In some embodiments the soluble protein-targeting moiety is a protein inhibitor, a protein activator, a protein agonist, a protein antagonist and/or a protein binder.

Chimeric molecules comprising a soluble protein-targeting moiety may be particularly useful in the treatment of cancer. In some embodiments, the soluble protein target is selected from K-Ras, BRAF, NRAS, C-Myc and N-Myc. These proteins are oncogene products that are important in pathogenesis of many cancers. In some embodiments, the soluble protein-targeting moiety binds K-Ras, BRAF, NRAS, C-Myc or N-Myc.

In some embodiments, the soluble protein-targeting moiety is selected from the molecules shown in Table 2 below:

TABLE 2

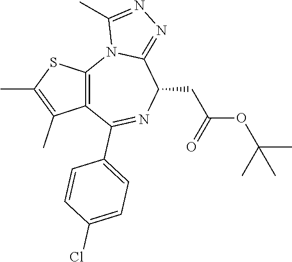 [SMILES: Cc1sc2c(c1C)C(c1ccc(Cl)cc1)=N[C@@H](CC(=O)OC(C)(C)C)c1nnc(C)n1-2]

(+)−JQ1 (binds BRD4)

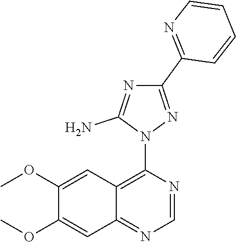 [SMILES: COc1cc2ncnc(-n3nc(-c4ccccn4)nc3N)c2cc1OC]

CP-466722 (binds ATM)

In some embodiments, the soluble protein-targeting moiety is a BRD4-targeting moiety.

Preferably, the soluble protein-targeting moiety is (+)-JQ1. (+)-JQ1 is the targeting moiety in the chimeric molecule NZ-147 described herein. (+)-JQ1 is a BRD4-targeting moiety.

(+)-JQ1 binds to the soluble protein BRD4. BRD4 is involved in enhancing c-MYC-dependent transcription, including the transcription of c-MYC itself. Thus, targeted degradation of BRD4 by a chimeric molecule of the invention comprising (+)-JQ1 targeting moiety may inhibit c-MYC-dependent transcription and thereby be useful in the treatment of cancer.

### Transcription Factor-Targeting Moieties

In some embodiments the targeting moiety is a transcription factor-targeting moiety. In some embodiments the transcription factor-targeting moiety is a protein inhibitor, a protein activator, a protein agonist, a protein antagonist, a protein binder and/or a peptide or peptidomimetic.

Chimeric molecules comprising a transcription factor-targeting moiety may be particularly useful in the treatment of cancer.

In some embodiments, the transcription factor-targeting moiety is selected from the molecules shown in Table 3 below:

TABLE 3

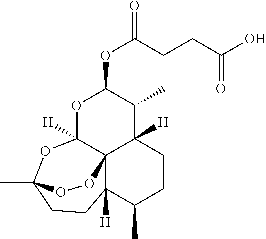 [SMILES: C[C@H]1[C@H](OC(=O)CCC(=O)O)O[C@@H]2O[C@@]3(C)CC[C@H]4[C@H](C)CC[C@@H]1[C@@]24OO3]

Artesunate (binds STAT3)

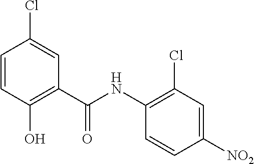 [SMILES: O=C(Nc1ccc([N+](=O)[O-])cc1Cl)c1cc(Cl)ccc1O]

Niclosamide (binds STAT3)

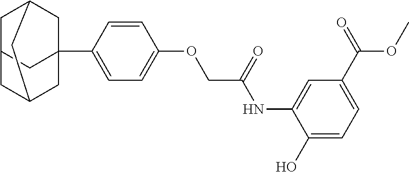 [SMILES: COC(=O)c1ccc(O)c(NC(=O)COc2ccc(C34CC5CC(CC(C5)C3)C4)cc2)c1]

LW6 (binds HIF1a)

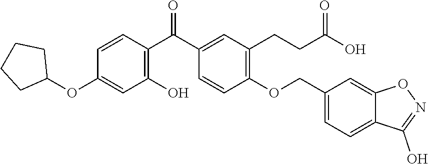 [SMILES: O=C(O)CCc1cc(C(=O)c2ccc(OC3CCCC3)cc2O)ccc1OCc1ccc2c(O)noc2c1]

T-5224 (binds AP-1)

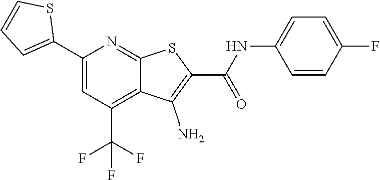 [SMILES: Nc1c(C(=O)Nc2ccc(F)cc2)sc2nc(-c3cccs3)cc(C(F)(F)F)c12]

FDI-6 (binds FOXM1)

### Protein Aggregate-Targeting Moieties

In some embodiments the targeting moiety is a protein aggregate-targeting moiety. In some embodiments the protein aggregate-targeting moiety is a covalent molecule, a protein inhibitor, a protein activator, a protein agonist, a protein antagonist, a protein binder, a protein-protein interaction inhibitor and/or a peptide or peptidomimetic.

Chimeric molecules comprising a protein aggregate-targeting moiety may be particularly useful in the treatment of neurodegenerative disorders characterised by protein aggregates, such as Parkinson's disease, ALS, Alzheimer's disease and Huntington's disease. In some embodiments, the protein aggregate-targeting moiety binds amyloid protein aggregates. In some embodiments, the protein aggregate-targeting moiety binds Huntingtin protein (htt) aggregates.

In some embodiments, the protein aggregate-targeting moiety is selected from the molecules shown in Table 4 below:

TABLE 4

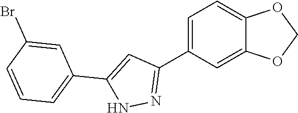 [SMILES: Brc1cccc(-c2cc(-c3ccc4c(c3)OCO4)n[nH]2)c1]

Anle138b

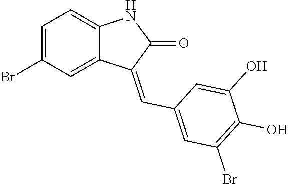 [SMILES: O=C1Nc2ccc(Br)cc2/C1=C/c1cc(O)c(O)c(Br)c1]

LC3-mHTT-IN-AN1 (binds LC3 and
htt aggregates)

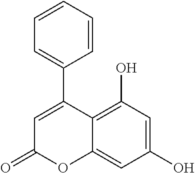 [SMILES: O=c1cc(-c2ccccc2)c2c(O)cc(O)cc2o1]

LC3-mHTT-IN-AN2 (binds
LC3 and htt aggregates)

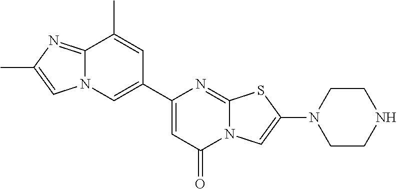 [SMILES: Cc1cn2cc(-c3cc(=O)n4cc(N5CCNCC5)sc4n3)cc(C)c2n1]

mHtt-IN-1 (binds mHtt)

### Peroxisome-Targeting Moieties

In some embodiments the targeting moiety is a peroxisome-targeting moiety. In some embodiments the peroxisome-targeting moiety is a covalent molecule, a protein inhibitor, a protein activator, a protein agonist, a protein antagonist, a protein binder, a protein-protein interaction inhibitor and/or a peptide or peptidomimetic.

### Virus-Targeting Moieties

In some embodiments the targeting moiety is a virus-targeting moiety. In some embodiments the virus-targeting moiety is a covalent molecule and/or an allosteric molecule. In some embodiments, the virus-targeting moiety binds to the virus capsid. In some embodiments, the virus-targeting moiety binds to virus genetic material.

In some embodiments, the virus target is human immunodeficiency virus (HIV). In some embodiments, the virus-targeting moiety binds to the HIV capsid.

### Protein Complex-Targeting Moieties

In some embodiments the targeting moiety is a protein complex-targeting moiety. In some embodiments the protein complex-targeting moiety is a covalent molecule, a protein inhibitor, a protein activator, a protein agonist, a protein antagonist, a protein binder, a protein-protein interaction inhibitor, an allosteric molecule and/or a peptide or peptidomimetic.

In some embodiments the protein complex-targeting moiety is MYCMI-6, which is an inhibitor of the Myc-Max complex and has the following structure:

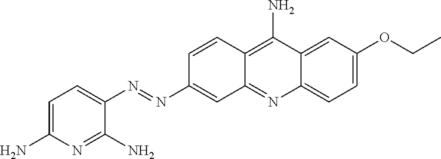 [SMILES: CCOc1ccc2nc3cc(/N=N/c4ccc(N)nc4N)ccc3c(N)c2c1]

The Myc-Max complex is a transcription factor which involved in cell cycle progression, cell differentiation and cell death. Targeting the Myc-Max complex for degradation may be useful in the treatment of cancer.

In some embodiments, the targeting moiety is a Myc-Max complex-targeting moiety. In other words, in some embodiments, the targeting moiety binds the Myc-Max complex. In some embodiments, the targeting moiety is a Max-targeting moiety. In some embodiments, the targeting moiety binds to Max. In some embodiments, the targeting moiety is a Myc-targeting moiety. In some embodiments, the targeting moiety binds to Myc.

### Lipid Droplet-Targeting Moieties

In some embodiments the targeting moiety is a lipid droplet-targeting moiety. In some embodiments, the lipid droplet-targeting moiety is a lipid droplet binder.

Chimeric molecules comprising a lipid droplet-targeting moiety may be particularly useful in the treatment of NBIA disorders, diabetes and obesity.

In some embodiments, the lipid droplet binder is selected from the molecules shown in Table 5 below:

TABLE 5

| | |
|---|---|
| 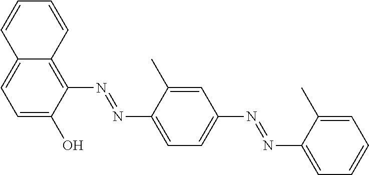 [SMILES: Cc1ccccc1/N=N/c1ccc(/N=N/c2c(O)ccc3ccccc23)c(C)c1] | Sudan IV |
| 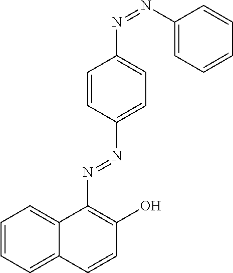 [SMILES: Oc1ccc2ccccc2c1/N=N/c1ccc(/N=N\c2ccccc2)cc1] | Sudan III |

c-MYC-Targeting Moieties

In some embodiments, the targeting moiety is a c-MYC-targeting moiety. In some embodiments, the c-MYC targeting moiety binds to c-MYC. c-MYC is a transcription factor. Therefore, a c-MYC targeting moiety is also a transcription factor-targeting moiety.

Chimeric molecules of the invention that comprise a c-MYC-targeting moiety target c-MYC for degradation, thereby potentially inhibiting proliferation of cancer cells and thus providing a cancer therapeutic.

In some embodiments, the c-MYC targeting moiety is selected from the molecules shown in Table 6 below:

TABLE 6

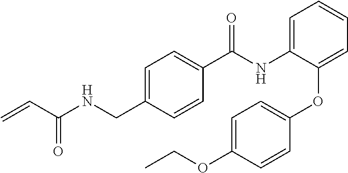 [SMILES: C=CC(=O)NCc1ccc(C(=O)Nc2ccccc2Oc2ccc(OCC)cc2)cc1]

EN4

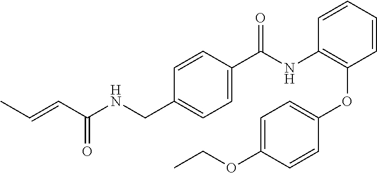 [SMILES: C/C=C/C(=O)NCc1ccc(C(=O)Nc2ccccc2Oc2ccc(OCC)cc2)cc1]

EN4-2

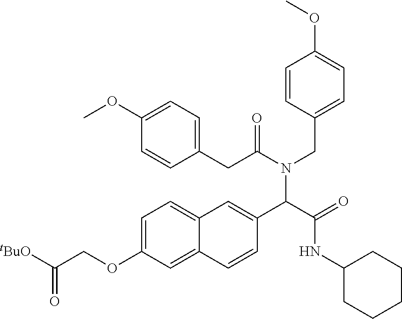 [SMILES: COc1ccc(CC(=O)N(Cc2ccc(OC)cc2)C(C(=O)NC2CCCCC2)c2ccc3cc(OCC(=O)OC(C)(C)C)ccc3c2)cc1]

NY2276

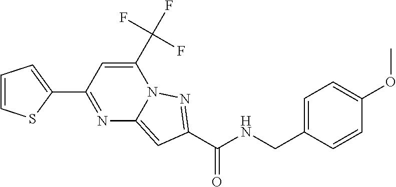 [SMILES: COc1ccc(CNC(=O)c2cc3nc(-c4cccs4)cc(C(F)(F)F)n3n2)cc1]

Mycro1

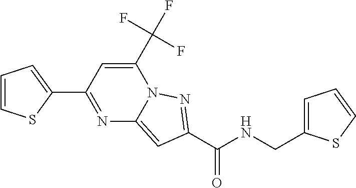 [SMILES: O=C(NCc1cccs1)c1cc2nc(-c3cccs3)cc(C(F)(F)F)n2n1]

Mycro2

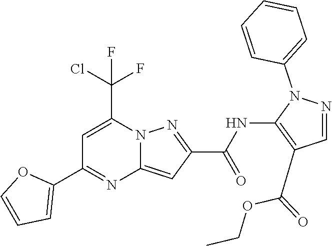 [SMILES: CCOC(=O)c1cnn(-c2ccccc2)c1NC(=O)c1cc2nc(-c3ccco3)cc(C(F)(F)Cl)n2n1]

Mycro3

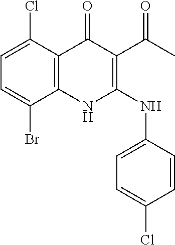 [SMILES: CC(=O)c1c(Nc2ccc(Cl)cc2)[nH]c2c(Br)ccc(Cl)c2c1=O]

KSI-3716

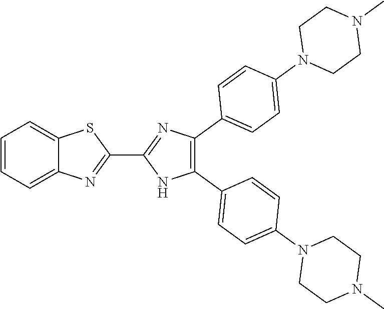 [SMILES: CN1CCN(c2ccc(-c3nc(-c4nc5ccccc5s4)[nH]c3-c3ccc(N4CCN(C)CC4)cc3)cc2)CC1]

IZTZ-1

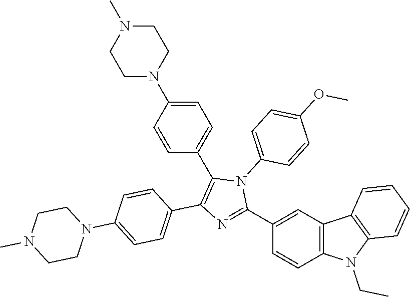 [SMILES: CCn1c2ccccc2c2cc(-c3nc(-c4ccc(N5CCN(C)CC5)cc4)c(-c4ccc(N5CCN(C)CC5)cc4)n3-c3ccc(OC)cc3)ccc21]

IZCZ-3

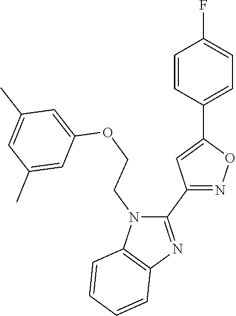 [SMILES: Cc1cc(C)cc(OCCn2c(-c3cc(-c4ccc(F)cc4)on3)nc3ccccc32)c1]

EP-12

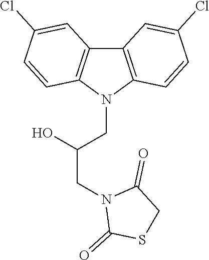 [SMILES: O=C1CSC(=O)N1CC(O)Cn1c2ccc(Cl)cc2c2cc(Cl)ccc21]

10074-A4

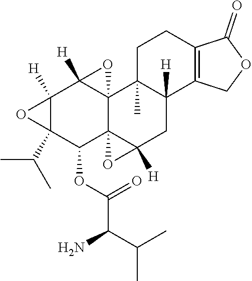

WBC100 (14-D-Valine-TPL)

In some embodiments, the c-MYC-targeting moiety is a derivative of EN4-2.

Preferably, the c-MYC-targeting moiety is the following derivative of EN4-2:

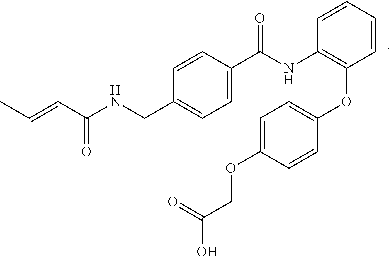 [SMILES: C/C=C/C(=O)NCc1ccc(C(=O)Nc2ccccc2Oc2ccc(OCC(=O)O)cc2)cc1]

The above derivative of EN4-2 is the c-MYC-targeting moiety in the chimeric molecule NZ-175 described herein.

### Bacterium-Targeting Moieties

In some embodiments, the bacterium-targeting moiety binds a bacterial protein on the outer surface of a bacterium.

### Antibodies

In some embodiments, the targeting moiety is an antibody. In some embodiments, the targeting moiety is a single-chain variable fragment (scFv). Any of the types of targeting moiety described herein (i.e. mitochondrial-targeting moiety, peroxisome-targeting moiety etc.) may be an antibody. In some embodiments the targeting moiety is an aptamer.

### Other Cancer Targets

Chimeric molecules of the invention intended to treat cancer may be targeted to any target molecule that may be causative of cancer. Potential targets for chimeric molecules of the invention intended to treat cancer include:
targets that affect cell proliferation, such as CDK2, CDK1, CDK4, CDK6, CDK7, MAT1, Cyclins A, Cyclins B, Cyclins D, Cyclins H, Cyclins K, MPS and Aurora B,
targets involved in transcription, such as CDK9, CDK12, CDK13, E2F1, E2F2, E2F3, FOXM1, HDAC3, HDAC11, HDAC6, C-Myc, L-Myc, N-Myc, MAX, BRD4, c-JUN/c-FOS (AP-1), HIF1a, NF-κB and ETS1,
targets involved in DNA replication and/or the DNA replication stress response, such as ATR, WEE1, Chk1, POLζ, POLθ, POLβ and POLQ,
targets involved in cell growth and/or cell differentiation, such as VEGF, STAT3, β-catenin, SHP2, and
targets involved in apoptosis, such as BCL-2, BCL-XL, BCL-W, BCL-2-A1, MCL1 and CDK5.

### Autophagy-Inducing Moiety

The term “autophagy-inducing moiety” refers to the part of the chimeric molecule which induces autophagosome formation at the target, thereby inducing engulfment of the target in an autophagosome, leading to degradation of the target. “Autophagy-inducing moiety” is synonymous with “autophagy-activating moiety”. As an isolated molecule/compound (i.e. when not part of the chimeric molecule of the invention), the autophagy-inducing moiety can independently induce autophagy.

The expression “induce autophagy” means to initiate autophagosome formation i.e. to begin formation of a new autophagosome de novo, rather than recruiting existing autophagosomes or trafficking the target to an existing autophagosome.

The autophagy-inducing moiety induces autophagy by acting on the endogenous autophagy machinery of the cell. Thus, the autophagy-inducing moiety acts on an endogenous factor in the cell that induces autophagy (i.e. an endogenous autophagy factor). The mechanism by which the autophagy-inducing moiety induces autophagy is not particularly limited. The term “acts on” thus refers to the autophagy-inducing moiety interacting with and/or affecting an endogenous autophagy factor to induce autophagy. The autophagy-inducing moiety may recruit, bind to, activate, inhibit, agonise or antagonise an endogenous autophagy factor, so long as the ultimate effect is to induce autophagy. The term “recruits” means that the autophagy-inducing moiety interacts with the endogenous autophagy factor so as to locate the endogenous autophagy factor at the target.

Accordingly, an autophagy-inducing moiety may be any molecule that induces autophagy by acting on an endogenous autophagy factor (i.e. a factor which induces autophagy). Different autophagy-inducing moieties act on different endogenous autophagy factors. In some embodiments, the autophagy-inducing moiety acts on an endogenous autophagy factor selected from Unc51 Like autophagy activating Kinase 1 (ULK1), ULK2, PI3K class III, mTOR, P110 delta, FUNDC1, BNIP3, NIX, VPS34 or Beclin-1.

ULK1 Unc51 Like autophagy activating Kinase 1 (ULK1) is a kinase that, when active, phosphorylates downstream targets to induce autophagosome formation.

In some embodiments, the autophagy-inducing moiety acts on ULK1. In some embodiments, the autophagy-inducing moiety recruits ULK1 (i.e. the autophagy-inducing moiety is an ULK1-recruiting moiety or an ULK1 recruiter). In some embodiments, the autophagy-inducing moiety binds ULK1 (i.e. the autophagy-inducing moiety is an ULK1-binding moiety or an ULK1 binder). In some embodiments, the ULK1 binder is a non-functional ULK1 antagonist or inhibitor (i.e. a derivative of a ULK1 antagonist that has been rendered non-antagonistic or non-inhibitive by modifications whilst retaining its capacity to bind ULK1).

Preferably, the autophagy-inducing moiety agonises (i.e. activates) ULK1 (i.e. the autophagy-inducing moiety is an ULK1-agonising moiety, an ULK1 agonist, ULK1-activating moiety or ULK1 activator).

In some embodiments, the autophagy-inducing moiety is an ULK1 agonist selected from BL-918, a derivative of BL-918, LYN-1604 and a derivative of LYN-1604.

In some embodiments, the autophagy-inducing moiety is a derivative of the ULK1 agonist BL-918. BL-918 has the following chemical structure:

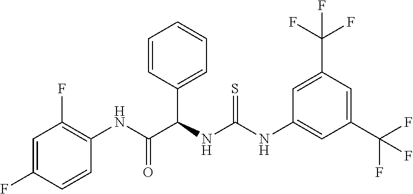 [SMILES: O=C(Nc1ccc(F)cc1F)[C@H](NC(=S)Nc1cc(C(F)(F)F)cc(C(F)(F)F)c1)c1ccccc1]

Preferably, the autophagy-inducing moiety is the ULK1 agonist NZ-60, which is a derivative of BL-918. NZ-60 has the following chemical structure:

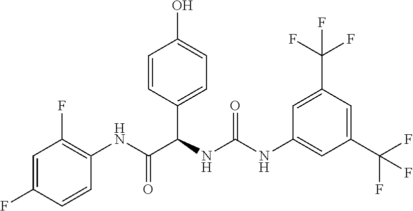 [SMILES: O=C(Nc1cc(C(F)(F)F)cc(C(F)(F)F)c1)N[C@@H](C(=O)Nc1ccc(F)cc1F)c1ccc(O)cc1]

10 LYN-1604 has the following chemical structure:

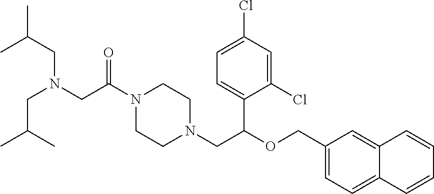 [SMILES: CC(C)CN(CC(=O)N1CCN(CC(OCc2ccc3ccccc3c2)c2ccc(Cl)cc2Cl)CC1)CC(C)C]

In another aspect, the invention provides an ULK1 agonist, which is NZ-60. In other words, the invention provides an ULK1 agonist, which has the following chemical structure:

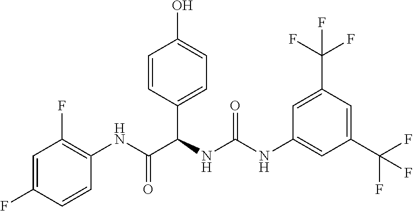 [SMILES: O=C(Nc1cc(C(F)(F)F)cc(C(F)(F)F)c1)N[C@@H](C(=O)Nc1ccc(F)cc1F)c1ccc(O)cc1]

### ULK1 Complex

ULK1 functions in a protein complex referred to as the ULK1 complex. In some embodiments, the autophagy-inducing moiety acts on a component of the ULK1 complex. In some embodiments, the autophagy-inducing moiety binds a component of the ULK1 complex. Components of the ULK1 complex include Atg13, Atg101 and FIP200. In some embodiments, the autophagy-inducing moiety binds Atg13. In some embodiments, the autophagy-inducing moiety binds Atg101. In some embodiments, the autophagy-inducing moiety binds FIP200.

### ULK2

ULK2 is a homologue of ULK1 which functions redundantly with ULK1 in induction of autophagosome formation.

In some embodiments, the autophagy-inducing moiety acts on ULK2. In some embodiments, the autophagy-inducing moiety recruits ULK2 (i.e. the autophagy-inducing moiety is an ULK2-recruiting moiety or ULK2 recruiter). In some embodiments, the autophagy-inducing moiety binds ULK2 (i.e. the autophagy-inducing moiety is an ULK2-binding moiety or an ULK2 binder). In some embodiments, the ULK2 binder is a non-functional ULK2 antagonist or inhibitor (i.e. a derivative of a ULK2 antagonist that has been rendered non-antagonistic or non-inhibitive by modifications whilst retaining its capacity to bind ULK1).

In some embodiments, the autophagy-inducing moiety agonises (i.e. activates) ULK2 (i.e. the autophagy-inducing moiety is an ULK2-agonising moiety, an ULK2 agonist, ULK2-activating moiety or ULK2 activator).

In some embodiments, the autophagy-inducing moiety is a non-inhibitive derivative of the ULK2 inhibitor SBI-0206965 or SBP-7455.

### PI3K Class III

In some embodiments, the autophagy-inducing moiety acts on PI3K class III. In some embodiments, the autophagy-inducing moiety recruits PI3K class III (i.e. the autophagy-inducing moiety is a PI3K class III-recruiting moiety or PI3K class III recruiter). In some embodiments, the autophagy-inducing moiety binds PI3K class III (i.e. the autophagy-inducing moiety is a PI3K class III-binding moiety or a PI3K class Ill binder). In some embodiments, the PI3K class III binder is a non-functional PI3K class III antagonist or inhibitor (i.e. a derivative of a PI3K class Ill antagonist that has been rendered non-antagonistic or non-inhibitive by modifications whilst retaining its capacity to bind PI3K class III).

In some embodiments, the autophagy-inducing moiety agonises (i.e. activates) PI3K class III (i.e. the autophagy-inducing moiety is an PI3K class Ill-agonising moiety, an PI3K class III agonist, PI3K class III-activating moiety or PI3K class III activator).

mTOR

In some embodiments, the autophagy-inducing moiety acts on mTOR. In some embodiments, the autophagy-inducing moiety inhibits (i.e. suppresses i.e. antagonises) mTOR (i.e. the autophagy-inducing moiety is an mTOR-antagonising moiety, mTOR antagonist, mTOR-inhibiting moiety, mTOR inhibitor, mTOR-suppressing moiety or mTOR suppressor).

In some embodiments, the mTOR inhibitor is selected from PF-04691502, limus compounds, such as sirolimus, tacrolimus and everolimus, rapamycin and torin. Many other mTOR inhibitors are known in the art.

PF-04691502 has the following chemical structure:

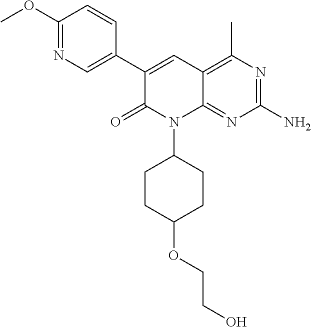 [SMILES: COc1ccc(-c2cc3c(C)nc(N)nc3n(C3CCC(OCCO)CC3)c2=O)cn1]

### P110 Delta

In some embodiments, the autophagy-inducing moiety acts on P110 delta. In some embodiments, the autophagy-inducing moiety inhibits (i.e. suppresses i.e. antagonises) P110 delta (i.e. the autophagy-inducing moiety is an P110 delta-antagonising moiety, P110 delta antagonist, P110 delta-inhibiting moiety, P110 delta inhibitor, P110 delta-suppressing moiety or P110 delta suppressor).

In some embodiments, the P110 delta inhibitor is idelalisib, which has the following chemical structure:

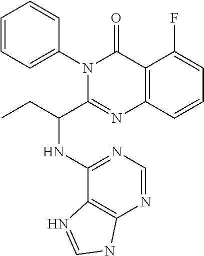 [SMILES: CCC(Nc1ncnc2[nH]cnc12)c1nc2cccc(F)c2c(=O)n1-c1ccccc1]

### FUNDC1

In some embodiments, the autophagy-inducing moiety acts on FUNDC1. In some embodiments, the autophagy-inducing moiety recruits FUNDC1 (i.e. the autophagy-inducing moiety is a FUNDC1-recruiting moiety or FUNDC1 recruiter). In some embodiments, the autophagy-inducing moiety binds FUNDC1 (i.e. the autophagy-inducing moiety is a FUNDC1-binding moiety or a FUNDC1 binder). In some embodiments, the FUNDC1 binder is a non-functional FUNDC1 antagonist or inhibitor (i.e. a derivative of a FUNDC1 antagonist that has been rendered non-antagonistic or non-inhibitive by modifications whilst retaining its capacity to bind FUNDC1). In some embodiments, the FUNDC1 binder is a peptide or peptidomimetic.

In some embodiments, the autophagy-inducing moiety agonises (i.e. activates) FUNDC1 (i.e. the autophagy-inducing moiety is an FUNDC1-agonising moiety, an FUNDC1 agonist, FUNDC1-activating moiety or FUNDC1 activator).

### BNIP3

In some embodiments, the autophagy-inducing moiety acts on BNIP3. In some embodiments, the autophagy-inducing moiety recruits BNIP3 (i.e. the autophagy-inducing moiety is a BNIP3-recruiting moiety or BNIP3 recruiter). In some embodiments, the autophagy-inducing moiety binds BNIP3 (i.e. the autophagy-inducing moiety is a BNIP3-binding moiety or a BNIP3 binder). In some embodiments, the BNIP3 binder is a non-functional BNIP3 antagonist or inhibitor (i.e. a derivative of a BNIP3 antagonist that has been rendered non-antagonistic or non-inhibitive by modifications whilst retaining its capacity to bind BNIP3). In some embodiments, the BNIP3 binder is a peptide or peptidomimetic.

In some embodiments, the autophagy-inducing moiety agonises (i.e. activates) BNIP3 (i.e. the autophagy-inducing moiety is an BNIP3-agonising moiety, an BNIP3 agonist, BNIP3-activating moiety or BNIP3 activator).

### NIX

In some embodiments, the autophagy-inducing moiety acts on NIX. In some embodiments, the autophagy-inducing moiety recruits NIX (i.e. the autophagy-inducing moiety is a NIX-recruiting moiety or NIX recruiter). In some embodiments, the autophagy-inducing moiety binds NIX (i.e. the autophagy-inducing moiety is a NIX-binding moiety or a NIX binder). In some embodiments, the NIX binder is a non-functional NIX antagonist or inhibitor (i.e. a derivative of a NIX antagonist that has been rendered non-antagonistic or non-inhibitive by modifications whilst retaining its capacity to bind NIX). In some embodiments, the NIX binder is a peptide or peptidomimetic.

In some embodiments, the autophagy-inducing moiety agonises (i.e. activates) NIX (i.e. the autophagy-inducing moiety is an NIX-agonising moiety, an NIX agonist, NIX-activating moiety or NIX activator).

### VPS34

In some embodiments, the autophagy-inducing moiety acts on VPS34. In some embodiments, the autophagy-inducing moiety recruits VPS34 (i.e. the autophagy-inducing moiety is an VPS34-recruiting moiety or VPS34 recruiter). In some embodiments, the autophagy-inducing moiety binds VPS34 (i.e. the autophagy-inducing moiety is an VPS34-binding moiety or an VPS34 binder). In some embodiments, the VPS34 binder is a non-functional VPS34 antagonist or inhibitor (i.e. a derivative of a VPS34 antagonist that has been rendered non-antagonistic or non-inhibitive by modifications whilst retaining its capacity to bind VPS34).

In some embodiments, the autophagy-inducing moiety agonises (i.e. activates) VPS34 (i.e. the autophagy-inducing moiety is an VPS34-agonising moiety, an VPS34 agonist, VPS34-activating moiety or VPS34 activator).

In some embodiments, the autophagy-inducing moiety is a non-inhibitive derivative of the VPS34 inhibitor VPS34-IN1, SAR405, Compound 19, PIK-III, autophinib or taselisib.

VPS34-IN1 has the following chemical structure:

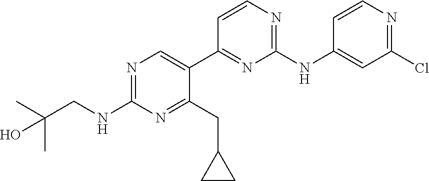 [SMILES: CC(C)(O)CNc1ncc(-c2ccnc(Nc3ccnc(Cl)c3)n2)c(CC2CC2)n1]

SAR405 has the following chemical structure:

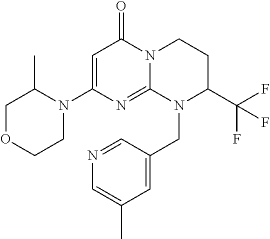 [SMILES: Cc1cncc(CN2c3nc(N4CCOCC4C)cc(=O)n3CCC2C(F)(F)F)c1]

Compound 19 has the following chemical structure:

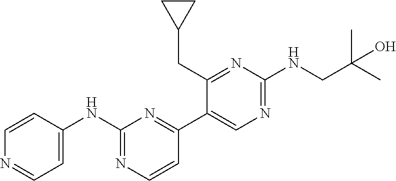 [SMILES: CC(C)(O)CNc1ncc(-c2ccnc(Nc3ccncc3)n2)c(CC2CC2)n1]

PIK-III (VPS34-IN2) has the following chemical structure:

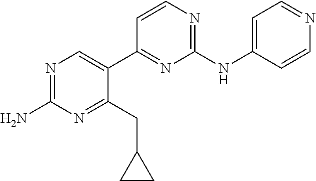 [SMILES: Nc1ncc(-c2ccnc(Nc3ccncc3)n2)c(CC2CC2)n1]

Autophinib has the following chemical structure:

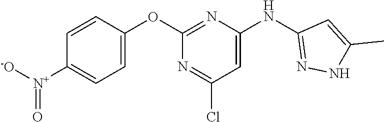

Taselisib has the following chemical structure:

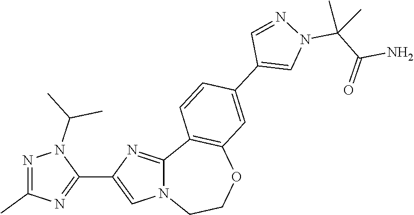 [SMILES: Cc1nc(-c2cn3c(n2)-c2ccc(-c4cnn(C(C)(C)C(N)=O)c4)cc2OCC3)n(C(C)C)n1]

In some embodiments, the autophagy-inducing moiety is 3-methyladenine.

### Beclin-1

In some embodiments, the autophagy-inducing moiety acts on Beclin-1. In some embodiments, the autophagy-inducing moiety recruits Beclin-1 (i.e. the autophagy-inducing moiety is a Beclin-1-recruiting moiety or Beclin-1 recruiter). In some embodiments, the autophagy-inducing moiety binds Beclin-1 (i.e. the autophagy-inducing moiety is a Beclin-1-binding moiety or a Beclin-1 binder). In some embodiments, the Beclin-1 binder is a non-functional Beclin-1 antagonist or inhibitor (i.e. a derivative of a Beclin-1 antagonist that has been rendered non-antagonistic or non-inhibitive by modifications whilst retaining its capacity to bind Beclin-1). In some embodiments, the Beclin-1 binder is a peptide or peptidomimetic.

In some embodiments, the autophagy-inducing moiety agonises (i.e. activates) Beclin-1 (i.e. the autophagy-inducing moiety is a Beclin-1-agonising moiety, Beclin-1 agonist, Beclin-1-activating moiety or Beclin-1 activator).

In some embodiments, the Beclin-1 agonist is Tat-beclin1, which is a peptide.

### AMPK

In some embodiments, the autophagy-inducing moiety acts on AMP-activated protein kinase (AMPK). In some embodiments, the autophagy-inducing moiety binds AMPK (i.e. the autophagy-inducing moiety is an AMPK-binding moiety or an AMPK binder). In some embodiments, the autophagy-inducing moiety activates AMPK (i.e. the autophagy-inducing moiety is an AMPK activator).

In some embodiments, the AMPK activator is metformin or phenformin.

Metformin has the following structure:

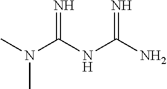

Phenformin has the following structure:

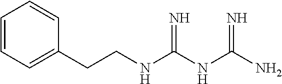 [SMILES: N=C(N)NC(=N)NCCc1ccccc1]

### Linker

The chimeric molecule may comprise a linker between the autophagy-inducing moiety and the targeting moiety. The linker, when present, connects the autophagy-inducing moiety to the targeting moiety.

The linker may be a polyethylene glycol (PEG) linker, an alkyl linker, an aromatic linker, a (mono) piperidine linker, a bi-piperidine linker, a (mono) piperazine or a piperidine-piperazine linker (in any arrangement of nitrogen).

A range of functional groups may be included at either end of the linker to couple the autophagy-inducing moiety and a targeting moiety to the linker. For example, each functional groups may be independently selected from ethers, alcohols, carboxylic acids, ureas, thioureas, amide, ester, azide, triazole, and amino-oxy groups.

The PEG linker may comprise a range of PEG units, for example 1-12 PEG units. In some embodiments, the linker is selected from 1 PEG unit, 2 PEG units, 3 PEG units, 4 PEG units, 5 PEG units, 6 PEG units, 7 PEG units, 8 PEG units, 9 PEG units, 10 PEG units, 11 PEG units and 12 PEG units. A PEG unit has the following structure:

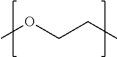

wherein the structure within square brackets is the repeating PEG unit.

Suitably, one or more PEG units may be triazole-linked. Suitably all of the PEG units are triazole-linked.

A preferable linker is the PEG linker shown in illustrative compound NZ-65. This PEG linker is shown below:

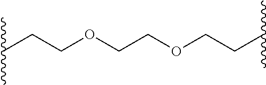 [SMILES: *CCOCCOCC*]

In some embodiments, the linker comprises the PEG linker depicted above. In some embodiments, the linker consists of the PEG linker depicted above.

Another preferable linker is the PEG linker in illustrative compounds NZ-147 and NZ-175. This PEG linker is shown below:

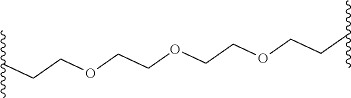 [SMILES: *CCOCCOCCOCC*]

In some embodiments, the linker comprises the PEG linker depicted above. In some embodiments, the linker consists of the PEG linker depicted above.

The linker may comprise a secondary amine group (NH) at one or both ends, as part of the attachment to the targeting moiety or autophagy-inducing moiety. An example of such a PEG linker is shown below:

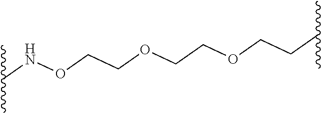 [SMILES: *CCOCCOCCON*]

25 In some embodiments, the linker comprises the PEG linker depicted above. In some embodiments, the linker consists of the PEG linker depicted above.

The alkyl linker may comprise a range of alkyl units, for example containing 1-12 carbons. In some embodiments, the linker is selected from a 1-carbon alkyl chain, a 2-carbon alkyl chain, a 3-carbon alkyl chain, a 4-carbon alkyl chain, a 5-carbon alkyl chain, a 6-carbon alkyl chain, a 7-carbon alkyl chain, a 8-carbon alkyl chain, a 9-carbon alkyl chain, a 10-carbon alkyl chain, a 11-carbon alkyl chain and a 12-carbon alkyl chain. Suitably, the linker is a 6-carbon alkyl chain. A 6-carbon alkyl chain linker is present in illustrative compound NZ-66.

in some embodiments, the aromatic linker is selected from a cyclohexane-based linker, a phenol-derived linker, an aniline-derived linker, an anilide-derived linker, a tolyl linker, a tosyl linker and a phenylacetate-derived linker.

A piperidine linker (i.e. mono-piperidine linker) is shown below:

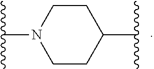 [SMILES: *C1CCN(*)CC1]

A bi-piperidine linker may comprise any suitable arrangement of nitrogen atoms. An illustrative bi-piperidine linker is shown below:

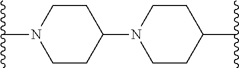 [SMILES: *C1CCN(C2CCN(*)CC2)CC1]

A piperazine linker (i.e. mono-piperazine linker) is shown below:

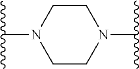 [SMILES: *N1CCN(*)CC1]

A piperidine-piperazine linker may comprise any suitable arrangement of nitrogen atoms. An illustrative piperidine-piperazine linker is shown below:

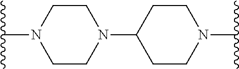 [SMILES: *N1CCC(N2CCN(*)CC2)CC1]

### Preferred Embodiments of the Chimeric Molecule

In a preferred embodiment, the chimeric molecule has the following structure chemical structure (this molecule may be referred to as NZ-65 herein):

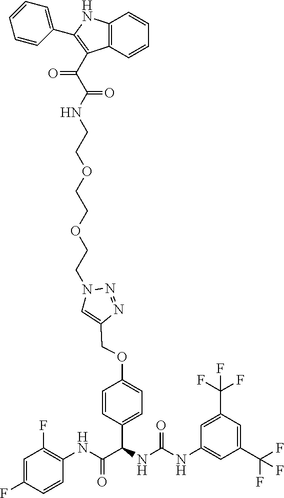 [SMILES: O=C(Nc1cc(C(F)(F)F)cc(C(F)(F)F)c1)N[C@@H](C(=O)Nc1ccc(F)cc1F)c1ccc(OCc2cn(CCOCCOCCNC(=O)C(=O)c3c(-c4ccccc4)[nH]c4ccccc34)nn2)cc1]

In a preferred embodiment, the chimeric molecule has the following structure chemical structure (this molecule may be referred to as NZ-66 herein):

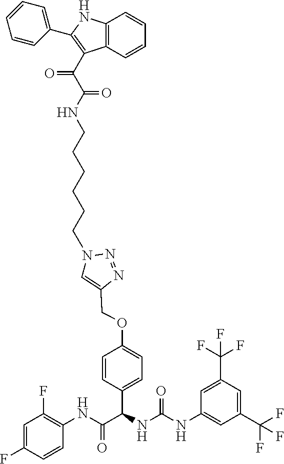 [SMILES: O=C(Nc1cc(C(F)(F)F)cc(C(F)(F)F)c1)N[C@@H](C(=O)Nc1ccc(F)cc1F)c1ccc(OCc2cn(CCCCCCNC(=O)C(=O)c3c(-c4ccccc4)[nH]c4ccccc34)nn2)cc1]

In a preferred embodiment, the chimeric molecule has the following structure chemical structure (this molecule may be referred to as NZ-147 herein):

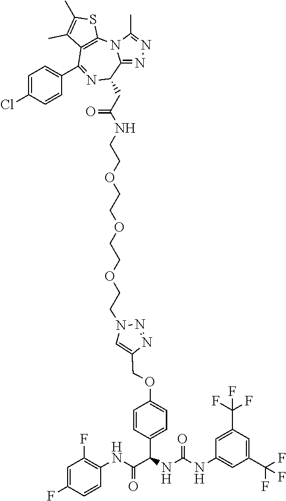 [SMILES: Cc1sc2c(c1C)C(c1ccc(Cl)cc1)=N[C@@H](CC(=O)NCCOCCOCCOCCn1cc(COc3ccc([C@@H](NC(=O)Nc4cc(C(F)(F)F)cc(C(F)(F)F)c4)C(=O)Nc4ccc(F)cc4F)cc3)nn1)c1nnc(C)n1-2]

In a preferred embodiment, the chimeric molecule has the following structure chemical structure (this molecule may be referred to as NZ-175 herein):

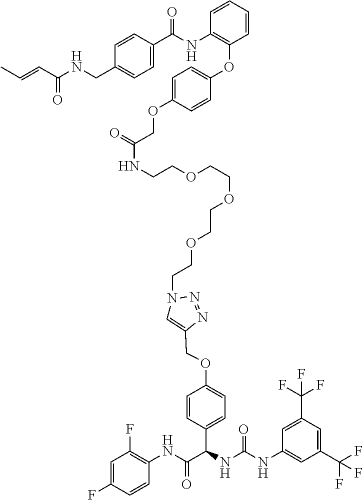 [SMILES: C/C=C/C(=O)NCc1ccc(C(=O)Nc2ccccc2Oc2ccc(OCC(=O)NCCOCCOCCOCCn3cc(COc4ccc([C@@H](NC(=O)Nc5cc(C(F)(F)F)cc(C(F)(F)F)c5)C(=O)Nc5ccc(F)cc5F)cc4)nn3)cc2)cc1]

### Pharmaceutical Composition

The present invention also relates to a pharmaceutical composition containing a molecule according to the present invention.

The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

### Methods of Treatment and Medical Uses

The present invention provides a method for treating and/or preventing a disease which comprises the step of administering a molecule of the present invention (for example in a pharmaceutical composition as described above) to a subject.

A method for treating a disease relates to the therapeutic use of the molecule of the present invention (for example in a pharmaceutical composition as described above). In this respect, the molecule be administered to a subject having an existing disease or condition in order to lessen, reduce or improve at least one symptom associated with the disease and/or to slow down, reduce or block the progression of the disease.

The method for preventing a disease relates to the prophylactic use of the molecule of the present invention (for example in a pharmaceutical composition as described above). In this respect, the molecule may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease.

It will be understood that the method of treatment may also be presented as a medical use i.e. the molecule of the invention (or a pharmaceutical composition comprising such a molecule) for use in the method of treatment or the use of the molecule of the invention (or a pharmaceutical composition comprising such a molecule) in the manufacture of a medicament for treating or preventing a disease in a subject.

### Diseases

Suitably, the disease may be any disease which can be treated by autophagy of a target as described herein.

Suitably, the disease may be selected from neurodegenerative disease, cancer, viral infection, cardiovascular disease, NIBA disorders, parasitic disease, diabetes and obesity.

Illustrative disease and associated targets applicable for the present invention are summarised in the following table:

| Disease | Associated target |
|---|---|
| Parkinson's disease | Mitochondria, Protein aggregates |
| ALS | Mitochondria, Protein aggregates (SOD1) |
| Alzheimer's Disease | Protein aggregates |
| Huntington's Disease | Protein aggregates (Htt) |
| Cardiovascular | Mitochondria |
| Disease | |
| Cancer (Solid and | Mitochondria (being investigated with |
| Haemotological) | NZ-65/66), Soluble proteins (being |
| | investigated with NZ-147), Transcription |
| | Factors (being investigated with NZ-147 |
| | and NZ-175), DNA |
| NBIA disorders | Mitochondria, Lipid Droplets, Iron reserves |
| HIV | Viral Capsid (in synthesis) |
| EBV | Viral Genetic material, Capsid, Infected nuclei |
| Other viral infections | Viral Genetic material, Capsid |
| Parasites | Parasite proteins, genetic material |
| Diabetes | Mitochondria, Lipid Droplets |
| Obesity | Lipid Droplets, Mitochondria |

Diseases where there may be mitochondrial dysfunction, and thus which may be treated by a chimeric molecule of the invention comprising a mitochondria-targeting moiety, include sepsis, obesity, organ transplantation, autoimmune disease, aging and diabetes, Down's syndrome, muscular dystrophy and ischemic stroke.

The neurodegenerative disease may be Parkinson's disease, Alzheimer's disease, amyotrophic lateral sclerosis (ALS), Huntington's disease, multiple sclerosis, Fronto-Temporal Dementia or Friedreich's Ataxia. Preferably, the neurodegenerative disease is Parkinson's disease.

The cancer may be selected from bladder cancer, gastric cancer, oesophageal cancer, breast cancer, colorectal cancer, cervical cancer, ovarian cancer, endometrial cancer, kidney cancer (renal cell), lung cancer (small cell, non-small cell and mesothelioma), brain cancer (e.g. gliomas, astrocytomas, glioblastomas), melanoma, lymphoma, small bowel cancers (duodenal and jejunal), leukemia, pancreatic cancer, hepatobiliary tumours, germ cell cancers, prostate cancer, head and neck cancers, thyroid cancer and sarcomas. In some embodiments, the cancer is breast cancer.

c-MYC-Dependent Cancers

In preferred embodiments, the disease is a c-MYC-dependent cancer. In other words, in preferred embodiments, the disease is a cancer wherein c-MYC is aberrantly expressed.

c-MYC is a transcription factor that is aberrantly expressed in many human cancers, where it drives proliferation of cancer cells. Chimeric molecules of the invention that comprise a c-MYC-targeting moiety, such as NZ-175 described herein, target c-MYC for degradation, thereby potentially inhibiting proliferation of cancer cells and thus providing a cancer therapeutic.

Likewise, chimeric molecules of the invention that target BRD4 (which enhances MYC expression), such as NZ-147 described herein, also provide potential cancer therapeutics.

A c-MYC-dependent cancer is a cancer in which c-MYC is aberrantly expressed (i.e., c-MYC is expressed at a higher level than in a non-cancerous cell (i.e., c-MYC overexpression), differently, or in a different form to its expression in non-cancerous cells).

More than 70% of cancers have aberrant expression of c-MYC. Hence, the chimeric molecules of the invention are likely to be relevant in most cancers, if not all. Specific cancers to which these compounds could be particularly relevant due to the high frequency at which MYC amplification is observed include: ovarian cancer (64%), esophageal cancer (45.3%) squamous lung cancer (37.2%) and breast cancer (30%) (Wang et al. 2021 Signal Transduction and Targeted Therapy 6 117, https://doi.org/10.1038/s41392-021-00500-y; Duffy et al. 2021 Cancer Treatment Review 94 102154, https://doi.org/10.1016/j.ctrv.2021.102154; Kalkat et al. 2017 Genes 8 (6) 151, https://www.mdpi.com/2073-4425/8/6/151).

In addition to amplification, alterations in c-MYC include chromosomal translocation, retroviral promoter insertion, activation of super enhancers, enhanced cell signalling, altered protein degradation and mutation. The cancer may be any of the following cancers: endometrial, non-small cell lung (including adenocarcinoma and squamous cell lung carcinoma), breast (including luminal A, luminal B, HER2+ and TNBC breast cancer), ovarian, oesophageal, liver, prostate, head-and-neck squamous cell carcinoma (HNSCC), liver, pancreatic, colorectal, neuroblastoma, B-cell lymphomas, renal clear cell carcinoma, adrenal cell carcinoma, medulloblastoma, and haematological cancers (especially T-ALL, B-ALL, AML, myeloma, and lymphoma, especially Burkitt's lymphoma (MYC-IgH+) and MYC positive DLBCL).

In preferred embodiments, the invention provides a method for treating and/or preventing cancer in a subject, wherein the method comprises the step of administering to a subject the following molecule:

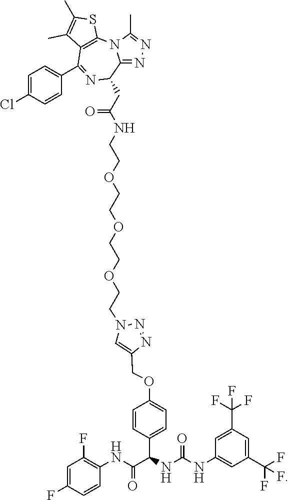 [SMILES: Cc1sc2c(c1C)C(c1ccc(Cl)cc1)=N[C@@H](CC(=O)NCCOCCOCCOCCn1cc(COc3ccc([C@@H](NC(=O)Nc4cc(C(F)(F)F)cc(C(F)(F)F)c4)C(=O)Nc4ccc(F)cc4F)cc3)nn1)c1nnc(C)n1-2]

In preferred embodiments, the invention provides a method for treating and/or preventing cancer in a subject, wherein the method comprises the step of administering to a subject the following molecule:

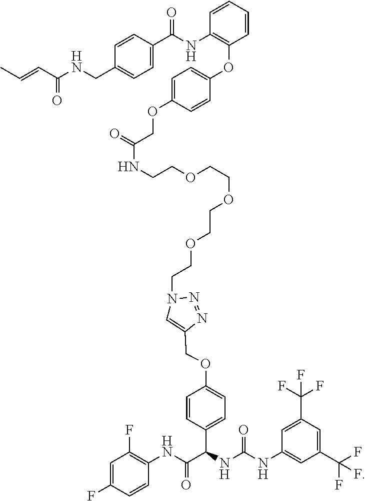 [SMILES: C/C=C/C(=O)NCc1ccc(C(=O)Nc2ccccc2Oc2ccc(OCC(=O)NCCOCCOCCOCCn3cc(COc4ccc([C@@H](NC(=O)Nc5cc(C(F)(F)F)cc(C(F)(F)F)c5)C(=O)Nc5ccc(F)cc5F)cc4)nn3)cc2)cc1]

### Precursor

The invention provides a chimeric molecule precursor comprising an autophagy-inducing moiety conjugated to an attachment group.

The autophagy-inducing moiety may be any autophagy-inducing moiety described herein.

An attachment group is any functional group that is suitable for attaching a targeting moiety or a linker using click chemistry. In some embodiments, the attachment group is an alkyne, amine, ester, ether, ketone, aldehyde, amide, alcohol (i.e. hydroxyl), carboxylic acid, urea, thiourea, isocyanate or isothiocynate group, or a sulphur functional group. Preferably, the attachment group is an alkyne group.

In some embodiments, the chimeric molecule precursor is the following molecule:

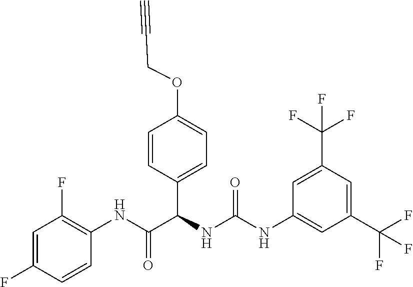 [SMILES: C#CCOc1ccc([C@@H](NC(=O)Nc2cc(C(F)(F)F)cc(C(F)(F)F)c2)C(=O)Nc2ccc(F)cc2F)cc1]

### Methods of Inducing Degradation

The invention provides a method of inducing degradation of a target in a cell, the method comprising introducing into the cell the chimeric molecule of the invention.

The methods of the invention of inducing degradation of a target in a cell encompass in vitro methods and in vivo methods. Thus, in some embodiments the invention provides an in vitro method of inducing degradation of a target in a cell, the method comprising introducing into the cell the chimeric molecule of the invention. The term “in vitro” means that the method is carried out upon a cell that is not in a living organism.

In some embodiments, the cell is a cultured cell. In some embodiments, the cell is from a cell line. In some embodiments, the cell is from a breast cancer cell line. In some embodiments, the cell is an MCF-7 cell. In some embodiments, the cell is a Triple-Negative Breast Cancer (TNBC) cell.

In some embodiments, the cell has been taken from a living organism (i.e. the cell is a sample from a living organism). In other words, the method is an ex vivo method. In some embodiments, the cell is a cancer cell. In some embodiments, the cell is a breast cancer cell.

In some embodiments, the invention is an in vivo method of inducing degradation of a target in a cell, the method comprising introducing into the cell the chimeric molecule of the invention. The term “in vivo” means that the method is carried out upon a cell that is in a living organism. In some embodiments, the cell is in a model organism. In some embodiments, the in vivo method is a non-therapeutic method.

The term “introducing” refers to any means by which the chimeric molecule may be transferred into the cell such that the molecule can associate with the target and induce autophagy. Techniques for introducing chimeric molecules into cells are known in the art. The exact technique used will vary depending on the type of chimeric molecule, as different types of molecule will require different techniques to cross the cell membrane. A person skilled in the art is able to select the appropriate technique for each chimeric molecule. Thus, in some embodiments, the chimeric molecule is introduced into the cell by micro-injection, nano-injection, electroporation, transfection or transduction. Some chimeric molecules can diffuse across the cell membrane or are moved across the cell membrane by active transport, and therefore in some embodiments the chimeric molecule is introduced to the cell by adding the chimeric molecule to cell culture medium.

As described herein, in some embodiments, the target is selected from mitochondria, a soluble protein, a transcription factor, a protein aggregate, peroxisomes, a virus, a protein complex, a lipid droplet and a bacterium. Preferably, the target is mitochondria, a soluble protein (such as BRD4) or a transcription factor (such as c-MYC). In some embodiments, the target is mitochondria. In some embodiments, the target is BRD4. In some embodiments, the target is c-MYC.

In some embodiments, the cell is selected from a neuronal cell, glial cell, microglial cell, haematopoietic stem cell, red blood cell, lymphocyte, platelet, cancer cell, cardiac smooth muscle cell, skeletal muscle cell, endothelial cell, epithelial cell, hepatocyte and pancreatic beta cell. In preferred embodiments, the cell is a neuronal cell or a cancer cell. In some embodiments, the cell is a neuronal cell. In some embodiments, the cell is a cancer cell.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

It must be noted that as used herein and in the appended claims, the singular forms “a”, “an”, and “the” include plural referents unless the context clearly dictates otherwise.

The terms “comprising”, “comprises” and “comprised of’ as used herein are synonymous with “including”, “includes” or “containing”, “contains”, and are inclusive or open-ended and do not exclude additional, non-recited members, elements or method steps. The terms “comprising”, “comprises” and “comprised of’ also include the term “consisting of.

The publications discussed herein are provided solely for their disclosure prior to the filing date of the present application. Nothing herein is to be construed as an admission that such publications constitute prior art to the claims appended hereto.

The invention will now be further described by way of Examples, which are meant to serve to assist one of ordinary skill in the art in carrying out the invention and are not intended in any way to limit the scope of the invention.

### Examples

### Materials and Methods

### Cell Culture and Chemicals

HEK293T (human embryonic kidney) cells (CRL-3216), A549 human non-small cell lung carcinoma (NSCLC) (CCL-185), PANC-1 human pancreatic ductal adenocarcinoma (PDAC) (CRL-1496), SH-SY5Y human neuroblastoma (CRL-2266) were purchased from ATCC. ULK1/2 WT MEF (SV40) (RRID: CVCL_5A51) 27, ULK1+ULK2 DKO MEF (SV40) (RRID: CVCL_5A57)²⁷ were gifted by Professor Sharon Tooze (The Francis Crick Institute, London, United Kingdom). HEK293T, A549, PANC-1 ULK1/2 WT MEF and ULK+ULK2 DKO MEF cells were cultured in High Glucose DMEM+GlutaMAX™+Pyruvate (ThermoFisher Scientific), supplemented with 10% foetal bovine serum (FBS) and 1% penicillin/streptomycin at 37° C./5% CO₂ (standard conditions). SH-SY5Y were cultured in High Glucose DMEM+GlutaMAX™ (ThermoFisher Scientific), supplemented with 10% FBS and 1% penicillin/streptomycin under standard conditions. The healthy control human fibroblasts and PD-PINK1 null culture containing a frameshift deletion in PINK1 (c.261_276del16;p.Y90L fsx12) were acquired from the UCL BioBank under Human Tissue Act Licence Number 12198 provided by, UCL Queen Square Institute of Neurology, University College London; cultured in High-glucose DMEM supplemented with 10% heat inactivated i FBS, 1% penicillin/streptomycin and 0.2 mM uridine. For mito-mKeima experiments, SH-SY5Y cells are cultured in the same media without phenol red. SH-SY5Y expressing mito-mKeima were produced as previously described^{28,29}. Cells were split at 80% confluency and media was refreshed every 2-3 days. Torin 1 (#475991) and Bafilomycin A1 (#B1793) were purchased from Sigma-Aldrich, dissolved in DMSO and stored at −20° C., Antimycin (#A8674) and Oligomycin (#75351) were purchased from Sigma-Aldrich and stored at −20° C. BL-918 (#S0819) and LYN-1604 (#S8597) were purchased from SelleckChem, dissolved in DMSO and stored at −80° C. AUTAC4 (#HY-134640) was purchased from MedChemExpress, dissolved in DMSO and stored at −80° C. NZ-65, NZ-66 were synthesised in house (See Chemistry); all of which, were dissolved in DMSO and stored at −80° C. or in lyophilised forms for long-term storage.

### Cell Culture in Example 8 (NZ-147) and Example 9 (NZ-175)

RPE1-hTERT-c-MYC-ER (Bertoli et al. 2016 Cell Reports 15 1412-1422 DOI: 10.1016/j.celrep.2016.04.036) were cultured in phenol-free Dublecco's Modified Eagle Medium F12 (DMEM F12) supplemented with 10% charcoal treated foetal bovine serum (ct-FBS), NaHCO₃ and 1% penicillin/streptomycin. A549 cells were cultured in DMEM, high glucose, GlutaMAX supplemented with 10% FBS, 1% penicillin/streptomycin. H1299 cells were cultured in RPMI 1640 supplement with 10% FBS, 1% penicillin/streptomycin. All cells were maintained at 37° C. in 95% O₂/5% CO₂.

All treatments with (+)-JQ1, NZ-147 or NZ-175 were performed at 100 nM concentration over 72 hours.

### Lentiviral Production

250,000 HEK293T cells were seeded into a 6-well plate. The subsequent day, cells were co-transfected with 900 ng psPAX2 (psPAX2 was a gift from Didier Trono (Addgene plasmid #12260; http://n2t.net/addgene: 12260; RRID: Addgene_12260)), 100 ng pMD2.G (pMD2.G was a gift from Didier Trono (Addgene plasmid #12259; http://n2t.net/addgene: 12259; RRID:Addgene_12259)) and 1 μg of pDEST-CMV mCherry-GFP-LC3B WT (Addgene plasmid #123230; http://n2t.net/addgene: 123230; RRID: Addgene_123230, Agrotis & Ketteler)³⁰ per well using X-tremeGene HP (Roche, #6366244001) at 2 μl/μg of total DNA. At 19 hours post-transfection, medium was replaced with complete DMEM. Viral supernatant was harvested at 48 hours post-transfection, filtered through a 0.22 μm low protein-binding syringe filter and stored at −80° C.

mCherry-EGFP-LC3 Tandem Reporter

PANC-1 and A549 cells were separately seeded at a medium density in 24-well plates and infected overnight with 500 μL of filtered viral supernatant containing polybrene at a final concentration of 8 μg/mL. Cells were selected at 48 h post-transfection with 1 μg/mL puromycin and expanded into 10 cm tissue-culture treated dishes. Puromycin treatment was withheld once control cells had died. Experiments were performed without puromycin present using pooled populations of transduced cells. A549 and PANC-1 stably expressing mCherry-EGFP-LC3 tandem reporter cells were imaged with the PerkinElmer Opera Phenix or Opera Phenix Plus High Content Imaging Systems and analysed using Columbus software (v2.9.1). Briefly, Hoechst 33342 was used to identify nuclei followed by determining regions of interest corresponding to cytoplasm for both EGFP and mCherry channels. This was followed by calculating the relative fluorescent intensity (RFI) of EGFP and mCherry for their respective cytoplasm. Spots were identified and gated for intensity greater than mean RSI for each channel under negative control conditions. Autophagosomes were defined as having EGFP and mCherry fluorescence. Autolysosomes were defined as only having autolysosome fluorescence. Excitation and emission wavelengths for each channel are as follows: Hoechst 33342=405/435-80 nm, EGFP=488/500-550 nm, mCherry=561/570-630 nm, respectively. Images captured with a water immersion lens at 40× magnification.

### Western Blotting

Cells were homogenised in protein lysis buffer consisting of 0.1 M Tris pH 8.0, 0.1 M NaCl, 5% glycerol, IGEPAL® CA-630 (#18896. Sigma-Aldrich) and 5 mM EDTA, 0.1 M N-ethyl maleimide (ThermoFisher, #23030) and protease inhibitor (Cell Signalling Technologies, #5871). Protein concentrations were determined using the BCA assay to ensure equal loading. After addition of loading buffer, protein samples were electrophoresed and transferred to Bio-Rad Trans-blot Turbo PVDF membranes using the Bio-Rad turbo-blot system and subsequently blocked with TBS with 0.05% Tween-20 (#P1379, Sigma-Aldrich) (TBS-T)+3% bovine serum albumin (BSA) (#A7906, Sigma-Aldrich). The membranes were immunoblotted with primary antibodies, overnight at 4° C. ULK1 (D8H5) Rabbit mAb (#0854) [1:1000], Phospho-ULK1 (Ser 317) Rabbit antibody (#37762) [1:1000], LC3B Rabbit antibody (#2775) [1:1000], TOM20 (D8T4N) Rabbit mAb (#42406) [1:1000] and, Phospho-Ubiquitin (Ser 65) (E2J6T) Rabbit mAb (#62082) [1:500] were obtained from Cell Signalling Technologies. Recombinant Anti-Vinculin Rabbit antibody (EPR8185) (#ab129002) [1:5000] and Anti-Mitofusin2 mouse antibody (6A8) (#ab56889) [1:1000] were purchased from Abcam. Anti-Actin mouse monoclonal antibody (#A1978) [1:1000] and Anti-rabbit p62/SQSTM1 (#P0067) [1:1000], were purchased from Sigma-Aldrich. Mono- and polyubiquitinylated conjugates monoclonal antibody (FK2) (BML-PW8810-0100) [1:500] was purchased from Enzo Life Sciences. Anti BRD4 (E2A7X) rabbit monoclonal antibody (#13440), Anti c-MYC (D84C12) rabbit monoclonal antibody (#5605) [1:1000], Anti GAPDH (D4C6R) mouse monoclonal antibody (#97166) [1:1000], Anti Max (S20) rabbit monoclonal antibody (#4739) [1:1000] were purchased from Cell Signalling Technologies. Anti Phospho-CHK1 (Ser 345) rabbit monoclonal antibody (#2341) [1:1000], Anti gamma-H2AX (Ser 139) rabbit monoclonal antibody (#2577) [1:1000] were obtained from Cell Signalling Technologies, Anti RPA32/RPA2 (phospho S4/S8) rabbit monoclonal antibody (ab87277) [1:1000] were obtained from Abcam.

Membranes were then incubated with horseradish peroxidase (HRP)-conjugated secondary antibodies (Rabbit #7074 and Mouse #7076) purchased from Cell Signalling Technologies for 1 hour and the HRP signal was detected using EZ-ECL Enhanced Chemiluminescence Detection Kit for HRP (#20-500-500, Sartorius), and imaged with an Alliance Q9 Advanced (UVITec). Where required, membranes were stripped with mild stripping buffer consisting of 0.2 M glycine, 3.47 mM SDS, 1% Tween 20, pH 2.2, blocked for 1 hour with TBS-T+3% BSA and re-probed with primary antibodies.

Imaging and Measuring Mitophagy with the Mito-mKeima Reporter

SH-SY5Y stably expressing mito-mKeima were produced as previously described28,29. Cells were seeded in 384 well CellCarrier Ultra plates (PerkinElmer) 24 hours before treatment in complete media without phenol red. Cells were treated with increasing concentrations of BL-918, AUTAC4, NZ-65 or NZ-66, 24 hours prior to treatment with increasing concentrations of mitochondrial uncouplers to cause mitochondrial insult. SH-SY5Y mitochondria were treated with DMSO, 3 μM CCCP, 10 μM CCCP, 0.1/0.1 μM antimycin/oligomycin (AO), 0.5/0.5 UM AO or 1/1 μM AO. Plates were imaged over an 18-hour period using a PerkinElmer Opera Phenix Plus HCS microscope and analysed using Columbus software. Hoechst 33342 dye was used to identify cell nuclei and mito-mKeima green was used to define the cytoplasm. The spot detection feature available in Columbus is then used to identify the number of spots and the area of the spots in the mito-mKeima green channel and mito-mKeima red channel in each cell. Spots are gated on their intensity being greater than the mean intensity for each respective channel under DMSO treated conditions. The mitophagy index is defined as the ratio of the total mito-mKeima red spot area to the total mito-mKeima green spot area. A higher mitophagy index is indicative of higher mitophagic activity. Excitation and emission spectra for each channel used were as follows: Hoechst 33342=405/435-480 nm, mito-mKeima green=488/650-760 nm, mito-mKeima red=561/570-630 nm, respectively. Images were captured with a water immersion lens at 63× magnification.

### Tetramethylrhodamine Methyl Ester (TMRM) Assay

SH-SY5Y cells were seeded in a 96 well PerkinElmer PhenoPlate 24 h prior to treatment with compounds. The following day, cells were incubated for 30 minutes with 3.24 UM Hoechst 33342 (#H3570, ThermoFisher), 100 nM TMRM and 20 nM MitoTracker Green (ThermoFisher Scientific). Cells were then treated with compounds, including 10 μM CCCP as a positive control before immediately imaging with a PerkinElmer Opera Phenix Plus High Content Screening system. Images were captured over the course of 18 hours and TMRM intensity in mitochondrial regions was quantified over time. TMRM intensity is directly proportional to changes in mitochondrial membrane potential (ΔΨₘ). Excitation and emission spectra for each channel are as follows: Hoechst 33342=405 nm/435-480 nm, MitoTracker Green=488/500-550 nm and TMRM=640/690-720 nm

### Immunofluorescence

For TOM20/ULK1 immunofluorescence, cells were seeded in a 384 well PerkinElmer CellCarrier Ultra plate 24 hours prior to treatment with compounds. Cells were treated for 18 hours after which they were fixed with 4% paraformaldehyde (PFA) in phosphate buffered saline (PBS) and blocked with 10% FBS and 0.1% Triton in PBS. Plates were incubated at 4° C. overnight, with 1:400 mouse anti-TOM20 (Santa Cruz, #sc-17764) and 1:400 rabbit anti-ULK1 (Cell Signalling Technologies, #0854). The following day, plates were incubated with 1:1000 goat anti-mouse Alexa 488 (ThermoFisher Scientific, #A-11029), 1:1000 goat anti-rabbit Alexa 568 (ThermoFisher Scientific, #A-11011) and 1.62 μM Hoechst 33342 at room temperature for 1 hour before imaging. For ATP5a/LC3 immunofluorescence, cells were fixed with ice-cold 1:1 MeOH: acetone and blocked with 3% BSA (Sigma-Aldrich, #A3803) in Dulbecco's PBS (D-PBS). Plates were incubated overnight at 4° C. with 1:400 LC3B (D11) XP Rabbit mAb Alexa Fluor 647 Conjugate (Cell Signalling Technologies, #65299) and 1:400 ATP5a (Cell Signalling Technologies, #18023) lightning-linked to Alexa Fluor 488. The following day, plates were incubated with 1.62 M Hoechst 33342 at room temperature for 1 hour before imaging. Excitation and emission spectra for each channel are as follows: Hoechst 33342=405/435-480 nm, Alexa 488=488/500-550 nm and Alexa 568=561/570-630 nm. Images were acquired at 40× and 63× magnification with water immersion lens.

### Immunofluorescence in Example 8 (NZ-147) and Example 9 (NZ-175)

RPE1-hTERT-c-MYC-ER cells were seeded in a 384 well PerkinElmer CellCarrier Ultra plate. In Example 8, cells were treated for 72 hours with DMSO, (+)-JQ1±4-OHT, or, NZ-147±4-OHT. In Example 9, cells were treated for 72 hours with 4-OHT, NZ-175, both, or neither. Thereafter, in both Examples 8 and 9, cells were fixed with 4% paraformaldehyde (PFA) in phosphate buffered saline (PBS) and blocked with 10% FBS and 0.1% Triton in PBS. Plates were incubated at 4° C. overnight, with 1:500 rabbit anti-c-MYC (D84C12) rabbit monoclonal antibody (#5605, Cell Signalling Technologies). The following day, plates were incubated with 1:1000 goat anti-rabbit Alexa 488 (ThermoFisher Scientific, #A-11008) and 1.62 μM Hoechst 33342 at room temperature for 1 hour before imaging. Excitation and emission spectra for each channel are as follows: Hoechst 33342=405/435-480 nm, Alexa 488=488/500-550 nm. Images were acquired at 40× and 63× magnification with water immersion lens.

### Plasmid Transfection and Imaging

pMRX-IP/Venus-mULK1 was a gift from Noboru Mizushima (Addgene plasmid #58743; http://n2t.net/addgene: 58743; RRID: Addgene_58743) 31. PANC-1 cells were seeded in antibiotic-free Opti-MEM media (ThermoFisher Scientific) in a PerkinElmer 96-well PhenoPlate before transfecting with 100 ng Venus-mULK1 in lipofectamine 3000 (ThermoFisher Scientific). 24 hours post-transfection, transfection media was replaced with normal culture media and cells were incubated for a further 24 hours. After a total transfection of 48 hours under standard conditions (see Cell Culture), compounds were added and subsequent image capture and/or recordings were acquired. 30 minutes prior to imaging, cells were incubated with 3.24 UM Hoechst 33342 and 25 nM Mito Tracker Deep Red (ThermoFisher Scientific). Images were captured using the PerkinElmer Opera Phenix Plus High Content Imaging System at 63× magnification with water immersion lens. Excitation and emission spectra for each channel used are as follows: Hoechst 33342=405/435-80 nm, Venus=488/515-550 nm, MitoTracker Deep Red=640/690-720 nm.

### Computational Modelling

The X-ray crystal structure of TSPO (PDB code: 2MGY) and the kinase domain of ULK1 (PDB code: 4WNP) were downloaded from the Protein DataBank (PDB). A set of conformers were generated for each ligand using the ETKDG method, which were each energy minimised using a Merck Molecular Force Field (MMFF), using RDKit in Python 3.8. Both proteins were prepared as standard, briefly, hydrogens were added, hydrogen bonds were optimised, and protonation states of histidine residues were assigned. For ULK1, the previously described putative binding pocket consisting of Arg18, Lys50, Asn86 and Tyr89⁸ was used to define the grid for docking. For TSPO, the binding pocket occupied by PK11195 in 2MGY was used to define the grid for docking. Conformational screening was performed using GOLD Docking and scored with the ChemPLP scoring function32 and protein-ligand interactions were analysed using BIOVIA Discovery Studio.

### Chemistry

All commercially available solvents and reagents were used without further treatment as received unless otherwise noted. Solvent evaporations were performed under reduced pressure (40-60° C.) by using a Buchi Rotavapor R-210. Reactions under an inert and anhydrous atmosphere were performed using commercial nitrogen. Thin layer chromatography (TLC) was performed with qualitative purposes on aluminium silica gel plates (Alugram Sil G/UV 254) with detection by UV light (>254 nm) charring with p-anisaldehyde, KMnO4, ninhydrin, phosphomolybdic acid or with cerium ammonium molybdate reagent [(NH₄) 6Mo7O₂₄·4H₂O; Ce(SO₄)₂ in a 1:10 mixture of concentrated H₂SO₄. in H₂O]. Column purifications were carried out in a Biotage Isolera Four Flash Chromatography System using the appropriate chromatography column (normal phase Biotage Sfär Silica 5 g/10 g/25 g and reverse phase Biotage Sfar C18 D-Duo 100 Å 12 g/30 g/60 g). 1H- and 13C-NMR spectra were performed at the UCL Chemistry NMR Facility with Bruker DRX 500 or 600 MHz spectrometers. Chemical shifts (0) are expressed in ppm relative to TMS and coupling constants (J) in Hz. J are assigned. DMSO-d₆, CDCl₃ and CD₃OD were used as solvents at room temperature except when indicated. Chemical shifts are calibrated using residual solvent signals (DMSO-d₆: δ(H)=2.50, δ(C)=39.52; CDCl₃: δ(H)=7.26, δ(C)=77.16; CD₃OD: δ(H)=3.31, δ(C)=49.00). NMR multiplicity abbreviations as follows: s=singlet, d=doublet, t=triplet, q=quartet, p=pentet, h=hextet; examples of complex multiplicities, dt=doublet of triplets, tdd=triplet of doublets of doublets. High-resolution mass spectrometry (HRMS) was performed via electron spray ionisation (ESI) using an ASAP-HESI ionisation connected to the Q Exactive Plus mass spectrometer and were performed at UCL Chemistry Mass Spectrometry Facility. LC-MS spectra were obtained using a single quadrupole LC/MSD XT mass spectrometer with electrospray ionisation (ESI), using an analytical C18 column (Kinetex 5 μm 100 Å, 50×4.6 mm) and a gradient 10%→95% MeCN+0.1% formic acid (FA) in H₂O+0.1% FA (6.5 min). Solvent abbreviations: H₂O=Water, DCM=dichloromethane, MeCN=acetonitrile, THF=tetrahydrofuran, Et₂O=diethyl ether, EtOAc=ethyl acetate, Cy=Cyclohexane.

2-oxo-2-(2-phenyl-1H-indol-3-yl) acetyl chloride

Synthesis has been previously described32 and was followed accordingly. Briefly, oxalyl chloride (0.620 mL, 7.22 mmol) was added dropwise to a stirring mixture of 2-phenylindole (1.2 g, 6.2 mmol) in dry Et₂O (20 mL) at 0° C. and stirred at room temperature for 4 hours. The resulting green precipitate was collected by vacuum filtration to obtain the desired product (1.6 g, 5.6 mmol, 90%) as a green solid.

N-(2-(2-(2-azidoethoxy) ethoxy)ethyl)-2-oxo-2-(2-phenyl-1H-indol-3-yl) acetamide (1)

Azido-PEG2-Amine (150 mg, 0.9 mmol) was dissolved in dry DCM (3 mL), the solution was cooled at 0° C. and NaHCO₃ (70 mg, 0.8 mmol) was added followed by 2-oxo-2-(2-phenyl-1H-indol-3-yl) acetyl chloride (200 mg, 0.7 mmol). After stirring at RT for 1 h, the solvent was removed under reduced pressure and the crude product was dissolved in EtOAc, washed with H₂O (×2), dried over MgSO₄, filtered and concentrated under reduced pressure. The crude product was purified by flash chromatography column on silica gel (EtOAc 50% in Cy) to yield the final compounds (150 mg, 0.4 mmol, 57%) as a green oil.

¹H-NMR (500 MHz, MeOD, J Hz) δ ppm 8.99 (s, 1H), 8.18 (d, 1H, J=7.70), 7.42 (q, 3H, J=6.44), 7.30 (quintet, 5H J=7.29), 6.80 (t, 1H, J=5.75), 3.25 (t, 2H, J=6.90), 3.11 (q, 2H, J=6.77), 1.57 (quintet, 2H, J=7.15), 1.46 (t, 2H, J=7.33), 1.39-1.21 (m, 4H).

¹³C-NMR (500 MHz, MeOD) δ ppm 186.87, 163.81, 147.44, 135.69, 132.46, 129.64, 129.08, 128.71, 128.21, 124.09, 123.10, 121.98, 111.37, 110.64, 51.48, 39.37, 29.81, 29.20, 28.84, 26.53, 26.49.

HRMS (ESI-MS) m/z calc, for C₂₂H₂₄N₅O₄ [M+H]⁺=422.17500, found 422.18228

N-(6-azidohexyl)-2-oxo-2-(2-phenyl-1H-indol-3-yl) acetamide (2)

6-Azido-hexylamine (0.06 mL, 4.3 mmol) was dissolved in dry DCM (1 mL), the solution was cooled at 0° C. and NaHCO₃ (35 mg, 0.4 mmol) was added followed by 2-oxo-2-(2-phenyl-1H-indol-3-yl) acetyl chloride (95 mg, 0.3 mmol). After stirring at RT for 1 h, the solvent was removed under reduced pressure and the raw material was dissolved in EtOAc, washed with H₂O (×2), dried over MgSO₄, filtered and concentrated under reduced pressure. The crude product was purified by flash chromatography column on silica gel (EtOAc 50% in Cyclohexane) to yield the final compounds (77 mg, 0.2 mmol, 67%) as a green oil.

¹H-NMR (500 MHz, MeOD, J Hz) δ ppm 10.21 (s, 1H), 8.17 (d, 1H, J=8.00), 7.29-7.1 (m, 6H), 6.92 (dd, 3H, J=7.45, 21.81), 3.66-3.56 (m, 6H), 3.43 (t, 2H, J=5.10), 3.35 (t, 2H, J=4.83), 3.22 (q, 2H, J=5.12).

¹³C-NMR (500 MHz, MeOD) δ ppm 186.72, 171.31, 164.76, 147.76, 136.05, 132.05, 129.37, 129.11, 128.95, 128.51, 128.27, 123.78, 122.93, 121.57, 112.14, 110.17, 77.43, 77.17, 76.92, 70.71, 70.50, 70.22, 69.31, 60.53, 50.78, 39.24, 27.05, 21.14, 14.31.

HRMS (ESI-MS) m/z calc. For C₂₂H₂₃N₅O₂ [M+H]⁺=390.18518, found 390.19245

(R)-4-(1-amino-2-((2,4-difluorophenyl)amino)-2-oxoethyl) phenyl isobutyl carbonate

To a solution of Boc-(R)-2-amino-2-(4-hydroxyphenyl) acetic acid (1.9 g, 7.3 mmol) in dry THF (25 mL) at −20° C., N-Methylmorpholine (1.6 mL, 15 mmol) and isobutyl chloroformate (1.9 mL, 15 mmol) were sequentially added. After stirring for 30 minutes at −20° C., 2,4-difluoroaniline (0.9 mL, 9.3 mmol) was added and the mixture was stirred overnight at RT. The solvent was removed under reduced pressure and the crude material was diluted with DCM, washed with 1M HCl (×1), 1M NaHCO₃(×1) and H₂O (×1). The organic layers were dried over MgSO₄, filtered concentrated under reduced pressure, purified by C18 reverse phase chromatography (5%→95% MeCN+0.1% formic acid in H₂O+0.1% formic acid) and dissolved in 4M HCl: 1,4-dioxane=1:1 (5 mL). After stirring at RT overnight, the solvent was removed under reduced pressure obtaining the corresponding deprotected amine as a white solid (450 mg, 1.2 mmol, 16%, 2 steps).

¹H-NMR (500 MHz, CDCl₃, J Hz) δ ppm 7.828-7.736 (m, 1H), 7.689 (d, 2H, J=8.223), 7.353 (d, 2H, J=8.637), 7.070-7.006 (m, 1H), 6.975 (d, 1H, J=9.240), 5.325 (s, 1H), 4.035 (d, 2H, J=6.547), 2.053-1.990 (m, 1H), 0.988 (d, 6H, J=6.744).

¹³C-NMR (500 MHz, CDCl₃) δ ppm 167.70, 154.88, 153.98, 131.76, 131.02, 130.97, 127.53, 127.45, 123.39, 112.31, 112.28, 112.13, 112.10, 105.31, 105.12, 105.10, 104.91, 76.05, 57.80, 57.48, 29.02, 19.11.

HRMS (ESI-MS) m/z calc, for C₁₉H₂₁N₂F₂O₄ [M+H]⁺=379.13911, found 379.14639

(R)-2-(3-(3,5-bis(trifluoromethyl)phenyl) ureido)-N-(2,4-difluorophenyl)-2-(4-hydroxyphenyl) acetamide

The previous product (160 mg, 0.42 mmol) was dissolved in dry MeCN (5 mL) and stirred with DIPEA (1 mL, 6 mmol) for 10 min at RT. 3,5-Bis(trifluoromethyl) phenyl isocyanate (0.2 mL, 1.2 mmol) was added dropwise, and the reaction mixture was stirred overnight at 50° C. The solvent was removed under reduced pressure and the crude material was diluted with DCM, washed with 1M HCl (×1), 1M NaHCO₃(×1) and H₂O (×1). The organic layers were dried over MgSO₄, filtered and concentrated under reduced pressure and used directly in the next step. The crude product (50 mg, 0.08 mmol) was dissolved in THF (1 mL) and cooled to 0° C. before adding 2M NaOH (1 mL). After stirring at RT overnight, the solvent was removed under reduced pressure and the crude material was diluted with EtOAc, washed with 1M HCl (×1), 1M NaHCO₃(×1) and H₂O (×1), dried over MgSO₄, filtered and concentrated under reduced pressure. The crude product was purified by C18 reverse phase chromatography (5% to 95% MeCN+0.1% formic acid in H₂O+0.1% formic acid) obtaining the desired product (35 mg, 0.07 mmol, 17%, 2 steps) as an off-white solid.

¹H-NMR (600 MHz, MeOD, J Hz) δ ppm 7.99 (s, 2H), 7.73 (td, 1H, J=8.89, 5.97), 7.47 (s, 1H), 7.36, (d, 1H, J=8.55), 6.985 (ddd, 1H, J=10.763, 8.763, 2.828), 6.905 (dddd, 1H, J=9.245, 8.061, 2.855, 1.439), 6.82 (d, 1H, J=8.55), 5.51 (s, 1H).

¹³C-NMR (600 MHz, MeOD) δ ppm 172.44, 162.20, 160.57, 158.88, 157.38, 156.29, 155.72, 143.02, 133.20, 129.79, 129.49, 127.51, 125.64, 123.84, 122.99, 118.86, 116.62, 115.62, 111.99, 104.90, 58.63.

HRMS (ESI-MS) m/z calc, for C₂₃H₁₆F₈N₃O₃ [M+H]+=534.09857, found 534.10584

(R)-2-(3-(3,5-bis(trifluoromethyl)phenyl) ureido)-N-(2,4-difluorophenyl)-2-(4-((1-(2-(2-(2-(2-oxo-2-(2-phenyl-1H-indol-3-yl) acetamido) ethoxy) ethoxy)ethyl)-1H-1,2,3-triazol-4-yl) methoxy)phenyl) acetamide [NZ-65] & (R)-2-(3-(3,5-bis(trifluoromethyl)phenyl) ureido)-N-(2,4-difluorophenyl)-2-(4-((1-(6-(2-oxo-2-(2-phenyl-1H-indol-3-yl) acetamido) hexyl)-1H-1,2,3-triazol-4-yl) methoxy)phenyl) acetamide [NZ-66]
(R)-2-(3-(3,5-bis(trifluoromethyl)phenyl) ureido)-N-(2,4-difluorophenyl)-2-(4-hydroxyphenyl) acetamide (35 mg, 0.07 mmol) was dissolved in dry MeCN and anhydrous K₂CO₃ (36 mg, 0.26 mmol) was added. After stirring for 10 min at RT, 80% propargyl bromide in toluene (0.025 mL, 0.33 mmol) was added and stirred at 80° C. for 48 h. The solvent was removed under reduced pressure and the crude material was diluted with EtOAc, washed with 1M HCl (×1), 1M NaHCO₃(×1) and H₂O (×1), dried over MgSO₄ filtered and concentrated under reduced pressure. The crude product was dissolved in THF:H₂O=9:1 (1 mL) followed by compound 1 or 2 (6 mg, 0.01 mmol), CuSO₄ (0.4 mg, 0.0025 mmol) and sodium ascorbate (1 mg, 0.005 mmol). After stirring at 50° C. overnight, the solvent was removed under reduced pressure and the crude product was purified by chromatography column on silica gel (5% MeOH in DCM) obtaining the final ULKTACs NZ-65 (10 mg, 0.010 mmol, 14%, 2 steps) and NZ-66 (11 mg, 0.011 mmol, 16%, 2 steps) as a yellow oil and yellow solid respectively.

### NZ-65:

¹H NMR (600 MHz, DMSO-d₆, J Hz) δ 12.38 (s, 1H), 8.53 (d, 1H, J=5.4), 8.19 (q, 1H, J=7.1), 8.08 (d, 1H, J=7.4), 7.55 (dd, 2H, J=4.2, 2.1), 7.52-7.41 (m, 5H), 7.32 (q, 1H, J=9.5), 7.24 (dt, 2H, J=21.8, 7.4), 7.06 (d, 1H, J=10.6), 6.80 (d, 1H, J=12.6), 6.64 (s, 1H), 5.63-5.54 (m, 1H), 5.11 (dd, 1H, J=27.9, 12.7), 4.52 (p, 1H, J=7.3), 4.37 (d, 1H, J=8.6), 4.34-4.30 (m, 1H), 4.25 (q, 1H, J=6.1), 4.18 (ddd, 2H, J=19.6, 9.2, 6.4), 3.83 (tdd, 3H, J=12.9, 9.5, 5.2), 3.50 (dt, 1H, J=7.4, 3.6), 3.42 (s, 2H), 3.38 (s, 4H), 3.28 (s, 1H), 3.18 (s, 2H), 2.88 (h, 2H, J=6.0).

¹³C NMR (600 MHz, DMSO-d₆) δ ppm 187.54, 176.70, 175.76, 175.70, 172.87, 170.90, 170.04, 166.66, 147.48, 135.78, 131.35, 129.63, 129.34, 128.07, 127.35, 123.36, 122.32, 120.97, 111.99, 109.27, 105.64, 105.46, 105.29, 93.12, 91.35, 88.23, 87.95, 87.92, 76.01, 75.08, 74.86, 74.83, 73.28, 73.13, 73.04, 72.22, 69.76, 69.44, 68.70, 68.22, 66.41, 50.13, 49.97, 49.46, 48.63, 40.01, 38.16, 37.88, 37.58.

HRMS (ESI-MS) m/z calc. For C₄₈H₄₁F₈N₈O₇ [M+H]⁺=993.88423, found 993.29650.

### NZ-66:

¹H NMR (600 MHz, DMSO-d₆, J Hz) δ 12.37 (s, 1H), 8.44 (d, 1H, J=5.7), 8.23 (dd, 1H, J=19.1, 4.1), 8.09 (d, 1H, J=7.7), 7.95 (dd, 1H, J=12.9, 10.5), 7.81-7.71 (m, 1H), 7.55 (d, 2H, J=4.1), 7.49-7.41 (m, 5H), 7.33 (ddd, 1H, J=13.9, 8.0, 3.1), 7.25 (dt, 2H, J=21.4, 7.5), 7.07 (d, 1H, J=8.8), 5.89 (d, 1H, J=4.7), 5.31 (q, 1H, J=3.0), 4.35 (t, 1H, J=7.1), 3.73 (dddt, 2H, J=15.4, 7.8, 5.6, 3.6), 2.71 (s, 2H), 2.42 (t, 1H, J=8.1), 1.91-1.62 (m, 11H), 1.17 (s, 4H).

¹³C NMR (600 MHz, DMSO-d₆) δ ppm 203.53, 203.20, 187.85, 177.91, 169.67, 166.45, 157.82, 153.92, 147.40, 135.79, 131.38, 129.62, 129.31, 128.05, 127.40, 123.34, 122.29, 120.97, 111.98, 109.31, 103.06, 101.88, 98.89, 97.18, 68.31, 66.40, 66.31, 66.14, 66.05, 65.73, 65.58, 52.17, 49.36, 40.96, 40.74, 40.24, 40.10, 40.04, 38.18, 37.58, 33.17, 32.01, 31.86, 31.75, 29.66, 28.10, 27.45, 25.82, 25.55, 23.33, 23.07, 23.00, 22.87, 22.19, 21.78.

HRMS (ESI-MS) m/z calc, for C₄₈H₄₁F₈N₈O₅ [M+H]+=961.88623, found 961.30667

### Synthesis of NZ-147

(S)-2-(4-(4-chlorophenyl)-2,3,9-trimethyl-6H-thieno[3,2-f][1,2,4]triazolo[4,3-a][1,4]diazepin-6-yl) acetic acid, also referred to as (+)-JQ1 carboxylic acid [(+)-JQ1-COOH)], was purchased from MedChemExpress. 21 mg (0.052 mmol, 1 equivalent) of (+)-JQ1-COOH) was added to an inert flask, charged with N₂, followed by 20.9 mg (0.055 mmol, 1.050 equivalents) of HATU and 12.6 mg (0.058 mmol, 1.1 equivalents) of 2-(2-(2-(2-azidoethoxy) ethoxy) ethoxy) ethan-1-amine. The reactants were dissolved in DMF before adding 20.3 mg (0.157 mmol, 3 equivalents) of diisopropylethylamine (DIPEA) and allowing to stir at room temperature overnight. The crude material was purified by C18 reverse phase column chromatography to produce 25 mg (0.042 mmol, 79.4% yield) (R)-(S)—N-(2-(2-(2-(2-azidoethoxy) ethoxy) ethoxy)ethyl)-2-(4-(4-chlorophenyl)-2,3,9-trimethyl-6H-thieno[3,2-f][1,2,4]triazolo[4,3-a][1,4]diazepin-6-yl) acetamide as a clear oil.

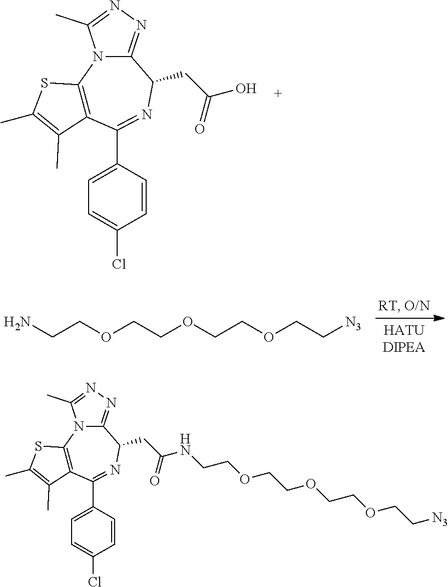 [SMILES: Cc1sc2c(c1C)C(c1ccc(Cl)cc1)=N[C@@H](CC(=O)O)c1nnc(C)n1-2 ; [N-]=[N+]=NCCOCCOCCOCCN ; Cc1sc2c(c1C)C(c1ccc(Cl)cc1)=N[C@@H](CC(=O)NCCOCCOCCOCCN=[N+]=[N-])c1nnc(C)n1-2]

5 mg (R)-2-(3-(3,5-bis(trifluoromethyl)phenyl) ureido)-N-(2,4-difluorophenyl)-2-(4-(prop-2-yn-1-yloxy)phenyl) acetamide (8.8 μmol, 1 eqv) and 5.8 mg(S)—N-(2-(2-(2-(2-azidoethoxy) ethoxy) ethoxy)ethyl)-2-(4-(4-chlorophenyl)-2,3,9-trimethyl-6H-thieno[3,2-f][1,2,4]triazolo[4,3-a][1,4]diazepin-6-yl) acetamide (9.6 μmol, 1.1 eqv) were dissolved in THF and added to a flask. 0.8 mg (5.3 μmol, 0.6 equivalents) of CuSO₄ dissolved in H₂O was then added, followed by 3.1 mg (15.8 μmol, 1.8 equivalents) of (+)-Sodium L-Ascorbate, to reduce copper in situ. This was heated to 50° C. and stirred overnight. The crude was flash purified by C18 reverse phase column chromatography to produce 10 mg (R)-2-(3-(3,5-bis(trifluoromethyl)phenyl) ureido)-2-(4-((1-(1-((S)-4-(4-chlorophenyl)-2,3,9-trimethyl-6H-thieno[3,2-f][1,2,4]triazolo[4,3-a][1,4]diazepin-6-yl)-2-oxo-6,9, 12-trioxa-3-azatetradecan-14-yl)-1H-1,2,3-triazol-4-yl) methoxy)phenyl)-N-(2,4-difluorophenyl) acetamide (8.5 μmol, 97.5% yield) as an orange oil. 1H NMR (600 MHz, Acetone) δ 8.15 (t, J=7.2 Hz, 2H), 8.06 (dddd, J=18.4, 12.1, 9.1, 4.3 Hz, 1H), 7.96 (s, 1H), 7.52-7.47 (m, 4H), 7.44-7.39 (m, 2H), 7.09-7.00 (m, 2H), 6.97 (t, J=8.7 Hz, 1H), 5.72 (d, J=6.4 Hz, 1H), 5.17 (d, J=3.7 Hz, 1H), 4.65 (t, J=6.9 Hz, 1H), 4.58 (t, J=4.1 Hz, 2H), 3.88 (t, J=4.9 Hz, 2H), 3.63-3.61 (m, 3H), 3.60-3.58 (m, 2H), 3.56-3.52 (m, 4H), 3.49 (d, J=5.6 Hz, 1H), 3.25 (q, J=7.3 Hz, 1H), 2.94 (s, 2H), 2.78 (s, 2H), 2.63 (d, J=11.4 Hz, 2H), 2.46-2.44 (m, 1H), 2.41 (s, 2H), 2.08 (s, 1H), 1.80-1.76 (m, 3H), 1.65 (d, J=5.6 Hz, 2H), 1.45-1.40 (m, 2H), 1.32 (t, J=7.3 Hz, 2H). 13C NMR (151 MHz, Acetone) δ 210.01, 198.14, 170.59, 164.45, 162.78, 159.33, 154.94, 154.28, 143.42, 138.09, 136.71, 132.43, 132.21, 131.11, 129.70, 129.25, 125.55, 125.38, 123.57, 118.19, 115.78, 114.77, 111.81, 111.64, 104.73, 104.57, 77.11, 71.04, 70.40, 69.93, 69.22, 68.04, 62.42, 58.22, 55.11, 54.72, 50.70, 46.59, 42.92, 39.90, 39.84, 38.95, 36.15, 31.00, 26.14, 23.32, 20.71, 18.50, 17.25, 14.51, 14.36, 12.94, 12.58, 11.76, 8.84.

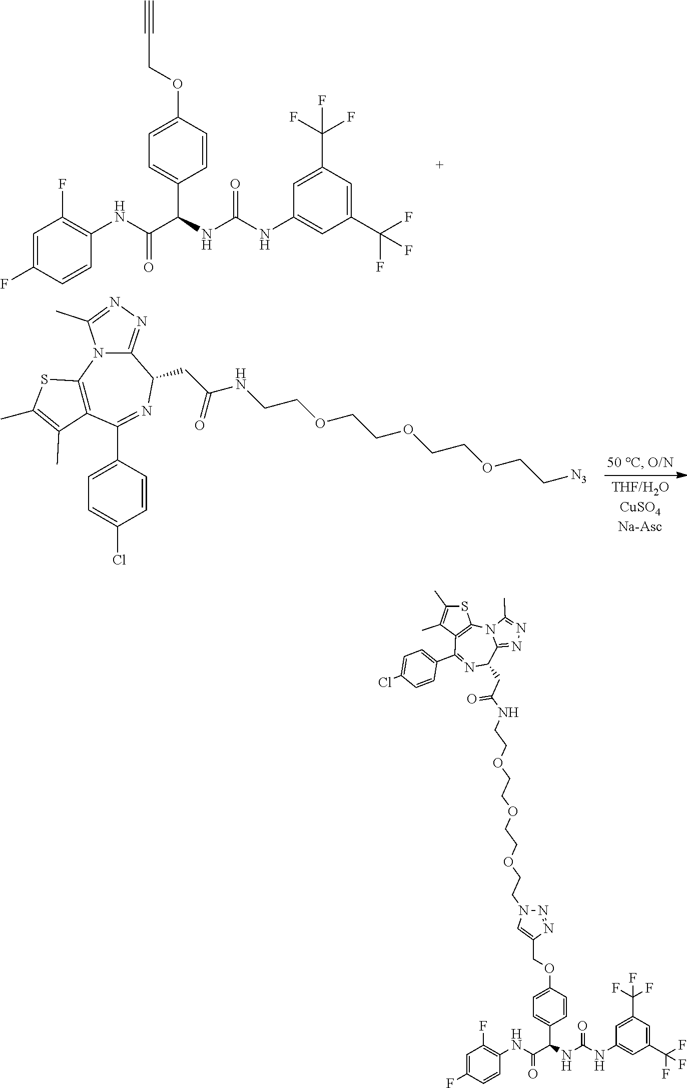 [SMILES: C#CCOc1ccc([C@@H](NC(=O)Nc2cc(C(F)(F)F)cc(C(F)(F)F)c2)C(=O)Nc2ccc(F)cc2F)cc1 ; Cc1sc2c(c1C)C(c1ccc(Cl)cc1)=N[C@@H](CC(=O)NCCOCCOCCOCCN=[N+]=[N-])c1nnc(C)n1-2 ; Cc1sc2c(c1C)C(c1ccc(Cl)cc1)=N[C@@H](CC(=O)NCCOCCOCCOCCn1cc(COc3ccc([C@@H](NC(=O)Nc4cc(C(F)(F)F)cc(C(F)(F)F)c4)C(=O)Nc4ccc(F)cc4F)cc3)nn1)c1nnc(C)n1-2]

### Synthesis of NZ-175

NZ-175 uses a modified derivative of EN4-2, a covalent ligand designed to target cysteine 171 on c-MYC. The structure of EN4-2 is shown below:

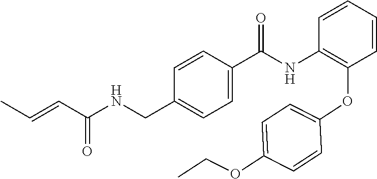 [SMILES: C/C=C/C(=O)NCc1ccc(C(=O)Nc2ccccc2Oc2ccc(OCC)cc2)cc1]

Methyl 4-aminomethyl benzoate hydrochloride (1 eqv) and triethylamine (2 eqv) were added to an inert flask, charged with N₂. This was stirred in crotonyl chloride (1 eqv), before adding an additional 1 equivalent of crotonyl chloride, dropwise, at 0° C. The reaction mixture was stirred for 16 hours at ambient temperature before diluting with EtOAc, washed with water, brine and dried over Na₂SO₄ and concentrated under reduced pressure. This was used immediately in the next step, without purification, to hydrolyse the methyl ester to the carboxylic acid. This was performed by dissolving 4-(crotonylamidomethyl) methyl benzoate in equal amounts of THF and H₂O and 4 equivalents of LiOH monohydrate were added at 0° C. This was stirred at ambient temperature for 4 hours, at which point 3M HCl was added until the solution reached pH 2-3. The reaction mixture was then extracted with EtOAc and the combined organic phases were washed with brine, dried over Na₂SO₄ and concentrated to give 4-(crotonylamidomethyl)benzoic acid. This was then purified by C18 reverse phase flash chromatography (5-95% MeCN:H₂O) to yield the product as an orange solid. ¹H NMR (600 MHz, DMSO) δ 12.69 (s, 1H), 8.54 (t, J=6.1 Hz, 1H), 7.89 (d, J=8.3 Hz, 2H), 7.35 (d, J=8.3 Hz, 2H), 6.71-6.62 (m, 1H), 5.98 (dd, J=15.3, 1.8 Hz, 1H), 4.39 (d, J=6.1 Hz, 2H), 1.80 (dd, J=6.9, 1.7 Hz, 3H). ¹³C NMR (151 MHz, DMSO) δ 167.22, 167.21, 165.02, 144.86, 138.41, 129.43, 129.38, 129.36, 129.33, 129.31, 129.26, 129.24, 127.30, 127.22, 127.15, 127.09, 127.01, 126.96, 126.94, 125.60, 41.78, 39.91, 39.77, 39.64, 39.50, 39.36, 39.22, 39.08, 17.45.

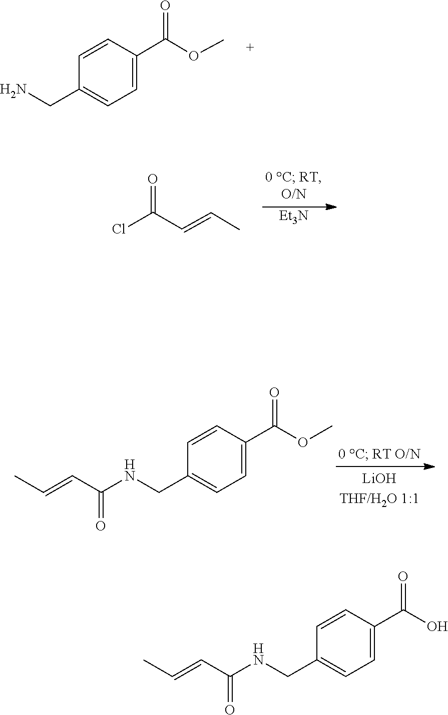

4-methoxyphenol (1 eqv) was added to a flask with potassium hydroxide (1.1 eqv) and heated to 150° C. until both reactants melted. This was stirred for 10 minutes at 150° C., after which, 1-chloro-2-nitrobenzene (1 eqv) was very carefully added (rapid addition results in steam ejection). The mixture was heated to 170° C. and stirred for an additional 2 hours. The mixture was cooled to room temperature and poured into a solution of 3% KOH dissolved in H₂O and stirred for an additional 2 hours at room temperature. This was cooled to 4° C. before vacuum filtrating to collect the crude product as a yellow solid. This was purified by recrystallising from ethanol to acquire 1-(4-Methoxyphenoxy)-2-nitrobenzene as yellow crystals.

1-(4-Methoxyphenoxy)-2-nitrobenzene was dissolved in acetic acid and 5 equivalents of iron powder before heating to 90° C. and stirring for 2 h to produce 2-(4-methoxyphenoxy) aniline. The reaction mixture was cooled to RT before neutralising with saturated NaHCO₃. This was then extracted with EtOAc, washed with NaHCO₃, and dried over Na₂SO₄. The combined organic extracts were concentrated under reduced pressure and used in the next step without further purification.

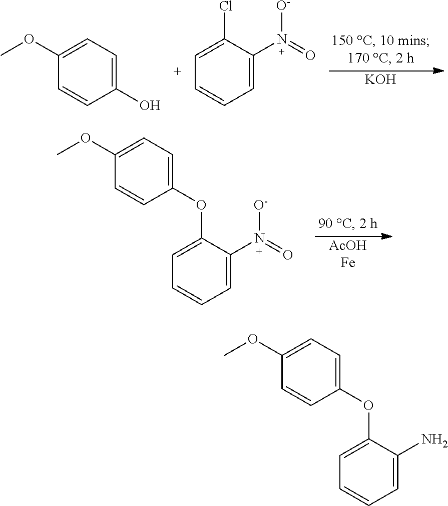

4-(crotonylamidomethyl)benzoic acid (1 eqv) and 2-(4-methoxyphenoxy) aniline (1.1 eqv) were added to an inert flask, charged with N₂ and cooled to 0° C. EDC (2 eqv) and 4-dimethylaminopyridine (1 eqv) were sequentially added to the flask before dissolving the reactants in dichloromethane. The flask was warmed to room temperature and allowed to stir overnight to produce 4-(crotonylamidomethyl)-N-(2-(4-methoxyphenoxy)phenyl) benzamide. The product was flash purified (DCM: MeOH). ¹H NMR (600 MHZ, CDCl₃) δ 6.99 (d, J=9.1 Hz, 3H), 6.90 (d, J=9.0 Hz, 2H), 6.85-6.82 (m, 2H), 6.73 (t, J=6.9 Hz, 1H), 3.93 (s, 1H), 3.81 (s, 3H). ¹³C NMR (151 MHZ, CDCl₃) δ 155.50, 150.83, 144.69, 138.30, 124.21, 119.19, 118.73, 116.42, 114.93, 77.55, 77.34, 77.12, 55.75.

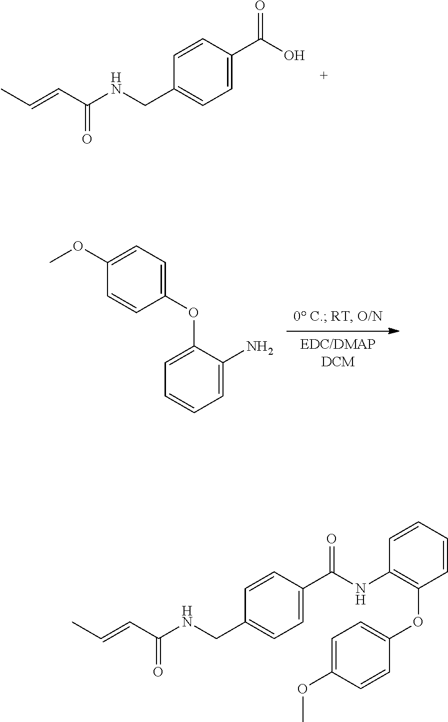 [SMILES: C/C=C/C(=O)NCc1ccc(C(=O)O)cc1 ; COc1ccc(Oc2ccccc2N)cc1 ; C/C=C/C(=O)NCc1ccc(C(=O)Nc2ccccc2Oc2ccc(OC)cc2)cc1]

4-(crotonylamidomethyl)-N-(2-(4-methoxyphenoxy)phenyl) benzamide was dissolved in 5 mL glacial acetic acid and 2.5 mL 48% HBr was added dropwise. This was heated to 100° C. and stirred for 75 minutes to produce a mixture of 4-(crotonylamidomethyl)-N-(2-(4-methoxyphenoxy)phenyl) benzamide, and the desired product 4-(crotonylamidomethyl)-N-(2-(4-hydroxyphenoxy)phenyl) benzamide. To acquire the product, the crude was flash purified by C18 reverse phase flash chromatography (5-95% MeCN:H₂O). ¹H NMR (600 MHz, MeOD) δ 7.92 (d, J=5.8 Hz, 1H), 7.78 (d, J=8.4 Hz, 2H), 7.37 (d, J=8.6 Hz, 2H), 7.11 (dtd, J=20.4, 7.5, 1.7 Hz, 3H), 6.67 (dd, J=8.1, 1.4 Hz, 2H), 6.64-6.61 (m, 3H), 5.98 (dd, J=15.3, 1.7 Hz, 1H), 4.47 (s, 2H), 1.85 (dd, J=6.9, 1.7 Hz, 3H).

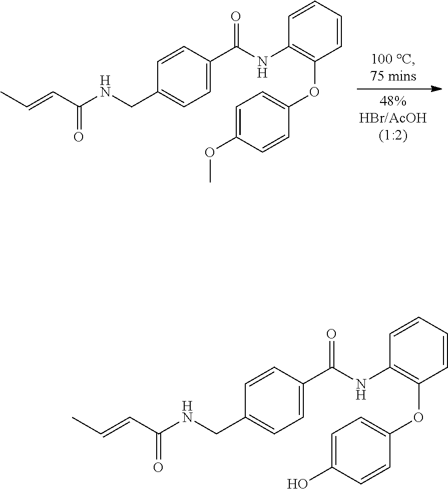 [SMILES: C/C=C/C(=O)NCc1ccc(C(=O)Nc2ccccc2Oc2ccc(OC)cc2)cc1 ; C/C=C/C(=O)NCc1ccc(C(=O)Nc2ccccc2Oc2ccc(O)cc2)cc1]

A solution of sodium chloroacetate was attained by cooling 1 g of chloroacetic acid to 0° C. and dissolved in 5 mL of H₂O. Solid NaOH was added until the pH reached 9-10, at which point sodium chloroacetate was produced. 4-(crotonylamidomethyl)-N-(2-(4-hydroxyphenoxy)phenyl) benzamide was dissolved in 1.25 mL of EtOH and added to a flask followed by the 5 mL solution of sodium chloroacetate dissolved in H₂O. The flask was heated to 105° C. and stirred at this temperature overnight. The EtOH and H₂O were both removed under reduced pressure before dry-loading the material for purification by C18 reverse phase column chromatography (60% MeCN:H₂O), producing 2-(4-(2-(4-(crotonylamidomethyl)benzamido) phenoxy) phenoxy) acetic acid. ¹H NMR (600 MHz, MeOD) δ 7.89 (d, J=7.2 Hz, 1H), 7.80-7.77 (m, 1H), 7.74 (d, J=8.2 Hz, 1H), 7.37 (t, J=7.8 Hz, 2H), 7.20-7.09 (m, 3H), 6.95 (t, J=2.9 Hz, 2H), 6.91 (d, J=2.8 Hz, 2H), 6.75 (d, J=8.9 Hz, 1H), 5.98 (dd, J=15.3, 1.8 Hz, 1H), 4.59 (d, J=17.0 Hz, 2H), 4.46 (s, 2H), 1.86 (dd, J=6.8, 1.7 Hz, 3H).

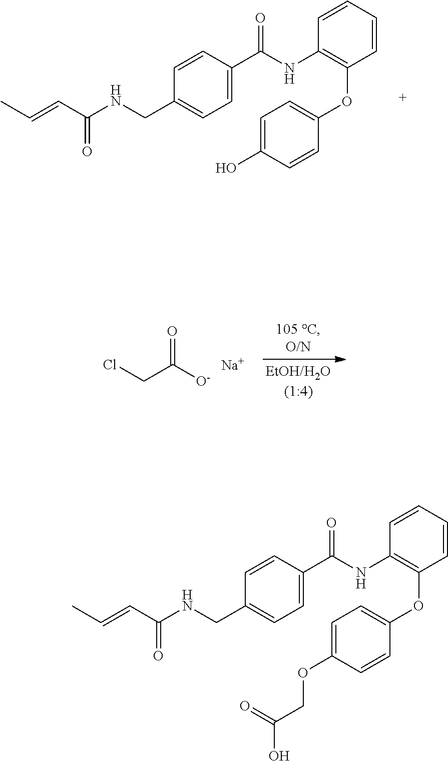 [SMILES: C/C=C/C(=O)NCc1ccc(C(=O)Nc2ccccc2Oc2ccc(O)cc2)cc1 ; O=C([O-])CCl ; [Na+] ; C/C=C/C(=O)NCc1ccc(C(=O)Nc2ccccc2Oc2ccc(OCC(=O)O)cc2)cc1]

2-(4-(2-(4-(crotonylamidomethyl)benzamido) phenoxy) phenoxy) acetic acid (1 eqv) was added to a flask containing EDC (2 eqv) and DMAP (1 eqv) at 0° C. This was warmed to room temperature and stirred for 10 minutes, before adding 2-(2-(2-(2-azidoethoxy) ethoxy) ethoxy) ethan-1-amine (1.1 eqv) and dissolving in DCM. This was stirred at room temperature overnight before flash purifying by C18 reverse phase column chromatography, producing 4-(crotonylamidomethyl)-N-(2-(4-((14-azido-2-oxo-6,9, 12-trioxa-3-azatetradecyl)oxy) phenoxy)phenyl) benzamide. ¹H NMR (600 MHz, MeOD) δ 8.34 (s, 1H), 7.89 (d, J=7.6 Hz, 1H), 7.75 (d, J=8.4 Hz, 2H), 7.36 (d, J=8.2 Hz, 2H), 7.21-7.14 (m, 2H), 7.00-6.96 (m, 5H), 6.92 (dd, J=7.8, 1.8 Hz, 1H), 6.82 (dq, J=15.3, 6.9 Hz, 1H), 5.98 (dd, J=15.2, 1.7 Hz, 1H), 4.46 (s, 2H), 3.60 (q, J=3.4, 2.9 Hz, 8H), 3.55 (t, J=5.5 Hz, 2H), 3.45 (t, J=5.4 Hz, 2H), 3.22 (d, J=4.9 Hz, 2H), 1.86 (dd, J=6.9, 1.7 Hz, 3H), 1.65 (s, 2H). ¹³C NMR (151 MHz, MeOD) δ 171.10, 168.55, 155.33, 152.60, 151.26, 144.40, 141.38, 134.49, 130.05, 128.76, 128.57, 127.51, 126.59, 125.79, 124.57, 120.65, 119.68, 119.62, 117.18, 117.13, 73.79, 71.57, 71.56, 71.45, 71.24, 71.08, 71.06, 70.38, 70.36, 68.78, 59.41, 59.39, 59.37, 51.68, 51.66, 49.38, 49.23, 49.09, 48.95, 48.81, 48.67, 48.52, 43.57, 39.92, 24.71, 20.68, 20.67, 20.66, 17.86, 13.90.

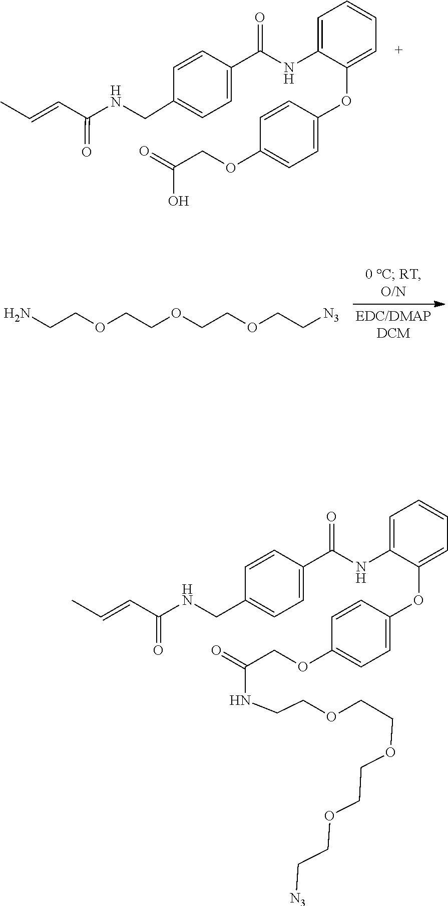 [SMILES: C/C=C/C(=O)NCc1ccc(C(=O)Nc2ccccc2Oc2ccc(OCC(=O)O)cc2)cc1 ; [N-]=[N+]=NCCOCCOCCOCCN ; C/C=C/C(=O)NCc1ccc(C(=O)Nc2ccccc2Oc2ccc(OCC(=O)NCCOCCOCCOCCN=[N+]=[N-])cc2)cc1]

(R)-2-(3-(3,5-bis(trifluoromethyl)phenyl) ureido)-N-(2,4-difluorophenyl)-2-(4-(prop-2-yn-1-yloxy)phenyl) acetamide (1 eqv) and 4-(crotonylamidomethyl)-N-(2-(4-((14-azido-2-oxo-6,9,12-trioxa-3-azatetradecyl)oxy) phenoxy)phenyl) benzamide (1.2 eqv) were dissolved in THF and added to a flask. CuSO₄ (0.6 eqv) and Na-Asc (1.8 eqv) were dissolved in H₂O, and separately, and sequentially added to the flask. This was heated to 50° C. and stirred overnight. This was purified by C18 reverse phase column chromatography to produce (R)-4-(crotonylamidomethyl)-N-(2-(4-((14-(4-((4-(1-(3-(3,5-bis(trifluoromethyl)phenyl) ureido)-2-((2,4-difluorophenyl)amino)-2-oxoethyl) phenoxy)methyl)-1H-1,2,3-triazol-1-yl)-2-oxo-6,9,12-trioxa-3-azatetradecyl)oxy) phenoxy)phenyl) benzamide [NZ-175]. ¹H NMR (600 MHz, MeOD) δ 8.19-8.13 (m, 1H), 8.10-8.06 (m, 1H), 8.00-7.95 (m, 2H), 7.91-7.85 (m, 2H), 7.73 (d, J=8.7 Hz, 2H), 7.49-7.44 (m, 2H), 7.33 (dd, J=8.5, 5.1 Hz, 2H), 7.14 (s, 2H), 7.02 (q, J=10.1 Hz, 3H), 6.95-6.86 (m, 8H), 6.75 (d, J=7.2 Hz, 1H), 5.97 (d, J=15.3 Hz, 1H), 5.55 (d, J=4.9 Hz, 1H), 4.77-4.69 (m, 2H), 4.50 (q, J=5.0, 4.2 Hz, 1H), 4.47-4.40 (m, 4H), 3.81-3.75 (m, 2H), 3.53-3.47 (m, 8H), 3.39 (d, J=5.4 Hz, 2H), 3.01-2.91 (m, 1H), 1.85 (ddd, J=6.9, 4.9, 1.7 Hz, 3H). ¹³C NMR (151 MHz, MeOD) δ 172.03, 168.55, 159.19, 156.22, 155.24, 152.82, 150.86, 144.39, 143.06, 141.40, 139.16, 133.22, 131.79, 130.46, 130.03, 129.84, 129.70, 129.51, 129.41, 128.77, 128.56, 127.49, 127.22, 126.55, 126.08, 125.78, 125.65, 125.31, 124.59, 123.85, 123.74, 121.36, 121.17, 120.62, 118.87, 118.23, 117.17, 116.52, 116.28, 116.20, 115.87, 115.65, 112.03, 104.91, 79.58, 76.85, 71.36, 71.30, 71.21, 70.31, 70.17, 68.75, 58.43, 56.56, 56.00, 51.40, 49.37, 49.23, 49.09, 48.95, 48.81, 48.66, 48.52, 43.56, 39.89, 35.35, 33.04, 30.83, 30.72, 23.71, 23.47, 21.29, 17.85, 14.41.

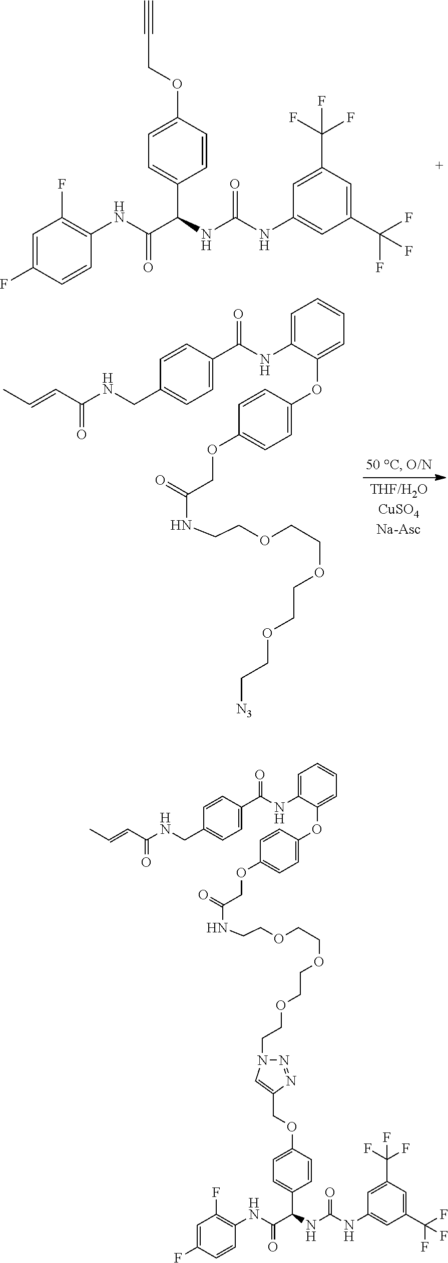 [SMILES: C#CCOc1ccc([C@@H](NC(=O)Nc2cc(C(F)(F)F)cc(C(F)(F)F)c2)C(=O)Nc2ccc(F)cc2F)cc1 ; C/C=C/C(=O)NCc1ccc(C(=O)Nc2ccccc2Oc2ccc(OCC(=O)NCCOCCOCCOCCN=[N+]=[N-])cc2)cc1 ; C/C=C/C(=O)NCc1ccc(C(=O)Nc2ccccc2Oc2ccc(OCC(=O)NCCOCCOCCOCCn3cc(COc4ccc([C@@H](NC(=O)Nc5cc(C(F)(F)F)cc(C(F)(F)F)c5)C(=O)Nc5ccc(F)cc5F)cc4)nn3)cc2)cc1]

### RNA Extraction and RT-qPCR

RNA was extracted with the Qiagen RNeasy Mini kit following manufacturers protocol. RT-qPCR SYBR assay was carried out with Mesa Blue following manufacturers instructions, 80 ng RNA per well, experimental triplicates, BioRad CTX Connect qPCR machine. RNA levels were normalized to GAPDH. Experiments were done in biological triplicate. The primers used were as follows:

| Gene Name | Forward Primer | Reverse Primer |
|---|---|---|
| GAPDH | 5′-GAA ATC CCA TCA CCA TCT | 5′-GAG CCC CAG CCT TCT CCA TG-3′ |
| | TCC AGG-3′ (SEQ ID NO: 1) | (SEQ ID NO: 2) |
| eIF4E | 5′-GGG AGG GTT GAT TGC CTA AA-3′ | 5′-GAC AAG TTG CCT GTG TGT |
| | (SEQ ID NO: 3) | TTA TT-3′ (SEQ ID NO: 4) |
| Cyclin E | 5′-TGT CCT GGA TGT TGA CTG CCT | 5′-TGT CGC ACC ACT GAT ACC |
| | TGA-3′ (SEQ ID NO: 5) | CTG AAA-3′ (SEQ ID NO: 6) |
| MDM2 | 5′-GAT CCA GGC AAA TCT GCA ATA | 5′-TGG TCT AAC CAG GGT CTC TT-3′ |
| | C-3′ (SEQ ID NO: 7) | (SEQ ID NO: 8) |
| PAICS | 5′-GAC AAG AGG AGA AGC AGA | 5′-TGA GGT ATT AGT CGC ATT T-3′ |
| | GAT TAG-3′ (SEQ ID NO: 9) | (SEQ ID NO: 10) |
| CDC45 | 5′-GAA GAG GAG ATA GTG GAG | 5′-GCT GAC GAT GTC CCA TGA |
| | CAA AC-3′ (SEQ ID NO: 11) | TAT T-3′ (SEQ ID NO: 12) |

### Colony Formation Assay

A549 and H1299 cells were seeded in 12 well plates at approximately 100 cells per well. In Example 8, wells were treated with DMSO, (+)-JQ1 or NZ-147. In Example 9, cells were treated with DMSO or NZ-175. Colonies were allowed to form over 120 h. Cells were fixed and stained with crystal violet staining solution, prepared in 70% ethanol.

### Cell Viability

A549 and H1299 cells were seeded in OptiPlate-384 well plates and treated with increasing concentrations of (+)-JQ1 or NZ-147 from 0 μM to 10 UM in Example 8 and with increasing concentrations of NZ-175 from 0 UM to 10 UM in Example 9. Cells were treated for 120 h before using the luciferase based CellTiter Glo assay (Promega) to measure cell viability.

### Example 1—Testing TSPO Ligand as Mitochondria-Targeting Moiety

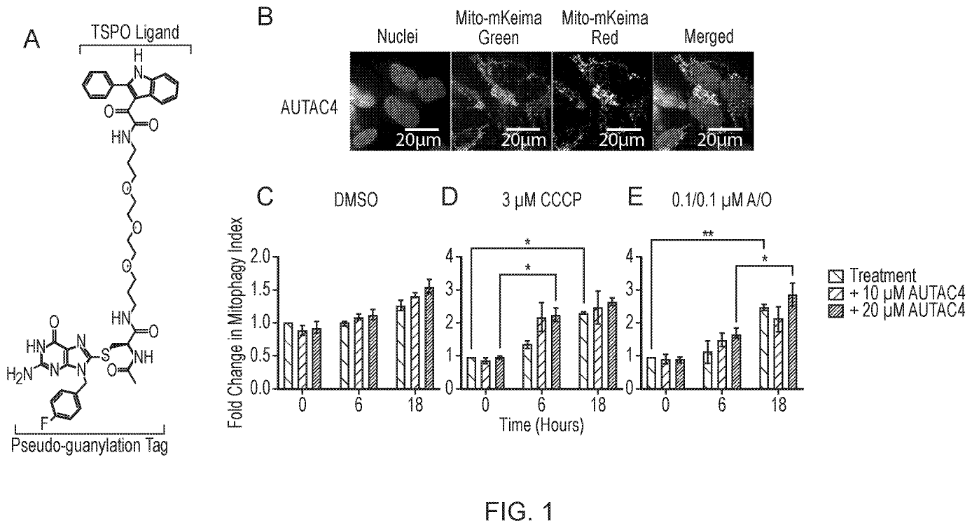

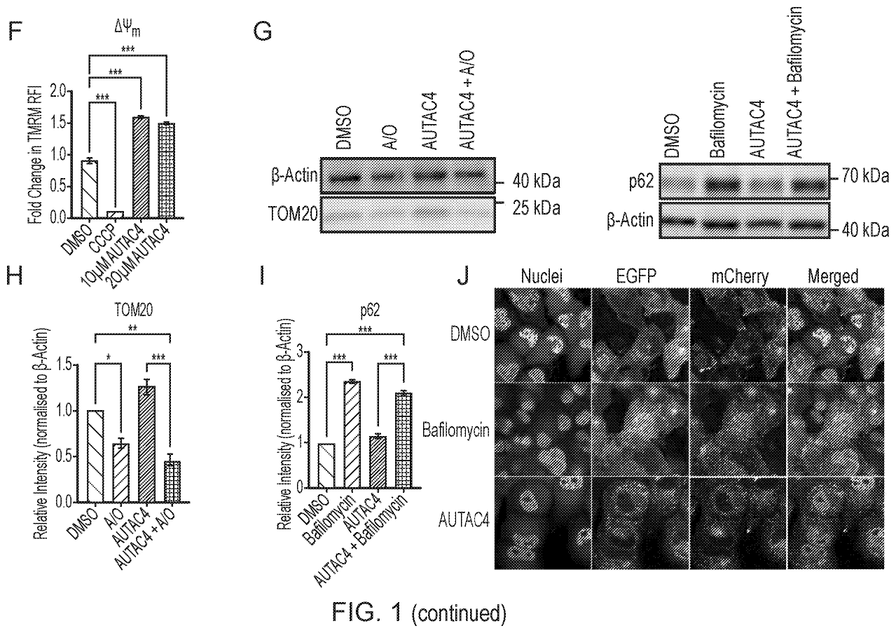

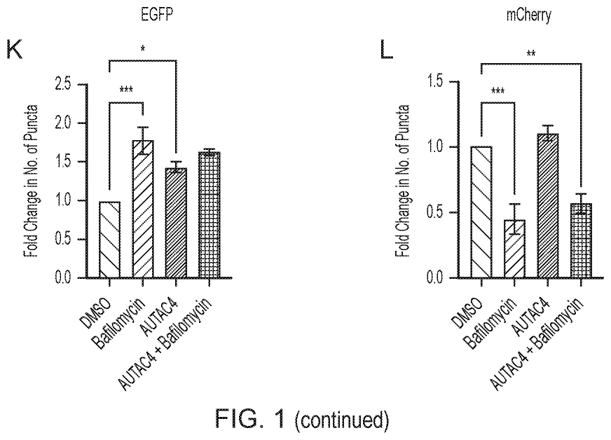

We investigated compounds that bind to and activate ULK1 as an autophagy inducer, for the design of an AUTAC. For the targeting warhead, we opted for a 2-phenylindole derivative TSPO ligand, which is known to bind to the translocator protein (also known as, peripheral benzodiazepine receptor) on the mitochondrial outer membrane³³⁻³⁵ and has been implemented in AUTAC design previously in the AUTAC4 chimeric molecule¹⁶ (structure shown in FIG. 1A). We tested AUTAC4 in various assays to explore its effect on autophagy and mitophagy, and to validate the suitability of the TSPO ligand as a mitochondria targeting warhead. We assessed the ability of AUTAC4 to induce mitophagy, as previously reported¹⁶. We used the mito-mKeima assay, which tags mitochondria with the mKeima fluorescent protein (FIG. 1B). Under neutral pH (e.g. in the cytoplasm), mito-mKeima is excited in the green fluorescence wavelength range, while in acidic conditions (as observed in lysosomes), it is excited by red light. We used the ratio of red over green puncta area to derive a mitophagy index (see Equation 1), directly proportional to mitophagic activity. Under basal conditions, we observed no effect on mitophagy by treatment with AUTAC4 (FIG. 1C). However, under conditions where mitochondria are damaged with mitochondrial toxins CCCP or antimycin/oligomycin (A/O), we observed an induction of mitophagy, that is mildly enhanced by co-treatment with AUTAC4 (FIG. 1D, E). To assess whether AUTAC4 might depolarise the mitochondrial membrane, we used the TMRM assay as described^{36,37} (FIG. 1F). We observed that AUTAC4 does not depolarise the mitochondrial membrane potential, and in contrast leads to hyperpolarisation (FIG. 1F). Thus, the induced mitophagy is not an artefact from increased depolarisation. The observed mitophagy when co-treated with A/O is also reflected when measuring the degradation of TOM20 by western blot (FIG. 1G, H). Taking these findings into consideration alongside the original authors' work, we determined that the TSPO ligand used in AUTAC-4 is suitable for mitochondrial targeting.

We also assessed whether AUTAC-4 influenced the formation of autophagosomes or autolysosomes using the mCherry-EGFP-LC3 tandem reporter assay, in combination with bafilomycin A1, a known inhibitor of autophagosome to lysosome fusion. Briefly, the tandem reporter exhibits EGFP and mCherry fluorescence in autophagosomes, and only mCherry fluorescence in autolysosomes as the EGFP is quenched due to the pH of the autolysosomes³⁰. We observed mild inductions of green fluorescent puncta, indicative of autophagosomes, upon treatment with AUTAC-4 but no effect on red fluorescent puncta, suggesting AUTAC-4 does not influence autolysosome activity (FIG. 1J-L), which was also secondarily validated by measuring the degradation of p62 by western blot (FIG. 11).

### Example 2—Testing LYN-1604 and BL-918 as Autophagy-Inducing Moieties

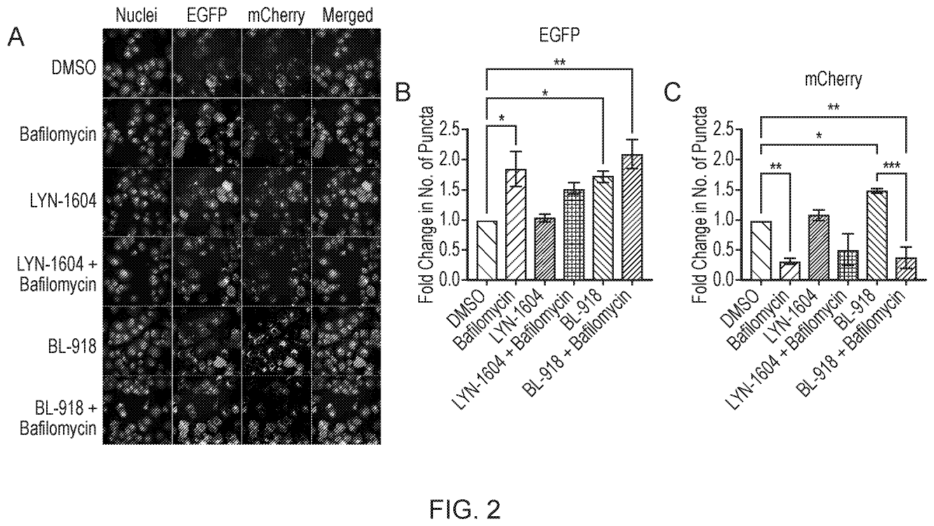

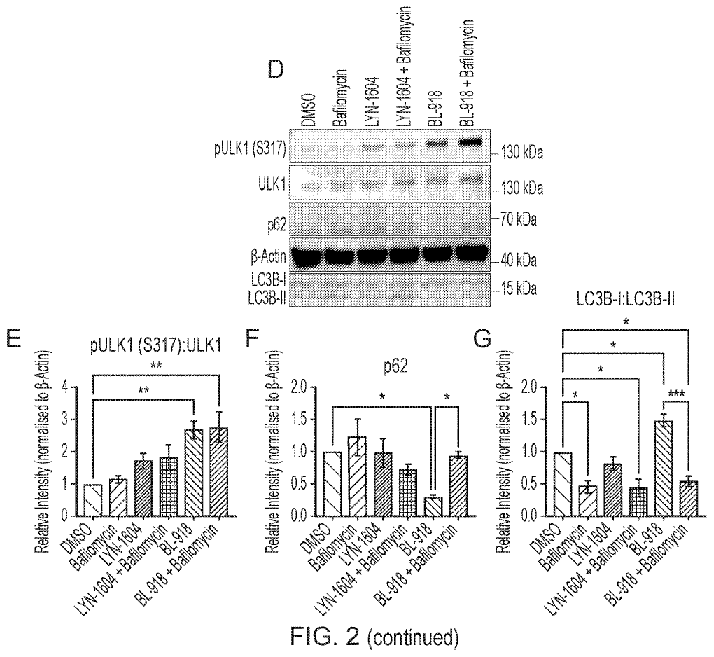

Next, we assessed currently known ULK1 agonists as potential autophagy activators for the design of ULKTACs. We tested the ability of two published ULK1 agonists, LYN-16047 and BL-9188 to activate autophagy using the tandem mCherry-EGFP-LC3 reporter. We observed a mild increase in autophagosome number upon treatment with BL-918, supporting its role as an activator of autophagy (FIG. 2A-C). We observed an induction of phosphorylation of ULK1 at Ser317, an activating phosphorylation, upon treatment with BL-918 (FIG. 2D-E) and a reduction in p62/SQSTM1 levels (FIG. 2F) with concomitant increases in the LC31: LC3II ratio (FIG. 2G), further supporting its role as an ULK1 activator. Overall, we confirm that BL-918 is a potent activator of ULK1 and autophagy and we decided to proceed with this compound in the design of our ULKTACs.

### Example 3—Synthesis of Chimeric Molecules NZ-65 and NZ-66

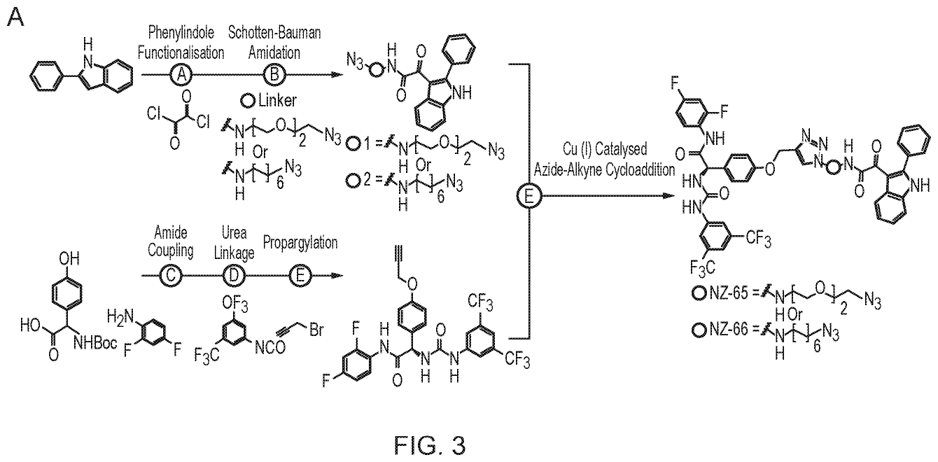

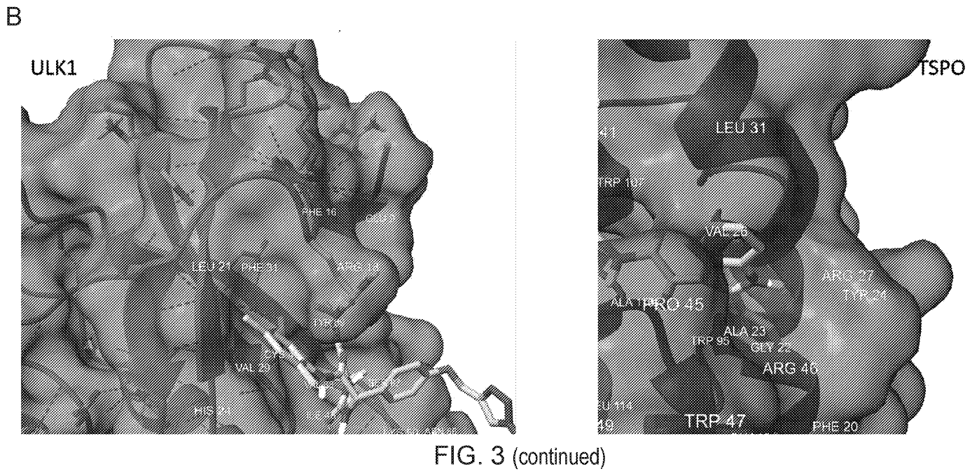

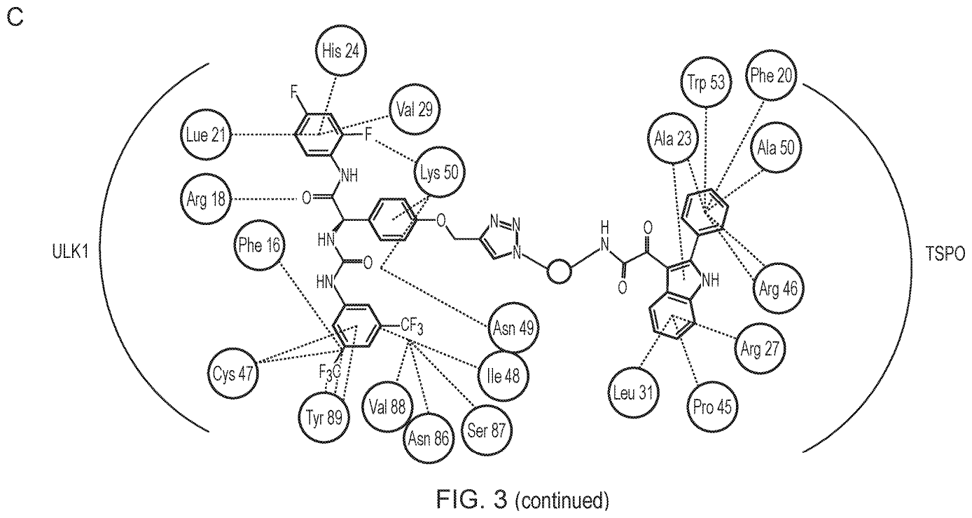

Next, we designed a chimeric compound composed of a BL-918 derivative (referred to as NZ-60 herein) as our autophagy activator linked to the TSPO ligand as our targeting warhead to the outer mitochondrial membrane, utilising Huisgen-Sharpless click chemistry for the key linkage step³⁸⁻⁴¹. We designed a chemical synthesis route in 9 steps to produce two ULKTACs, NZ-65 and NZ-66. NZ-65 employs a polyethylene glycol (PEG) linker while NZ-66 uses a six carbon hexane linker (FIG. 3A). The resulting molecules were >99% pure as verified by LCMS. Computational modelling shown in FIG. 3B suggests that the interactions between BL-918 and ULK1 are maintained with our compounds and ULK1 except for the T-sulfur interaction involving the thiourea in the parent molecule, as our compounds replace this with a urea functional group instead. Importantly, key interactions shown to be necessary for the activation of ULK1 in its kinase domain, namely, Arg18, Lys50, Asn86 and Tyr89⁸, appear to be maintained. When measuring the activation of ULK1 by NZ-65 and NZ-66 in an in vitro kinase assay measuring ATP consumption, we found comparable activity of both molecules versus BL-918 (Supplementary FIG. 1). Likewise, computational modelling also suggests typical interactions between the TSPO ligand and TSPO also remain unchanged^{42,43} (FIG. 3B).

### Example 4—Testing ULK1 Activation by NZ-65 and NZ-66

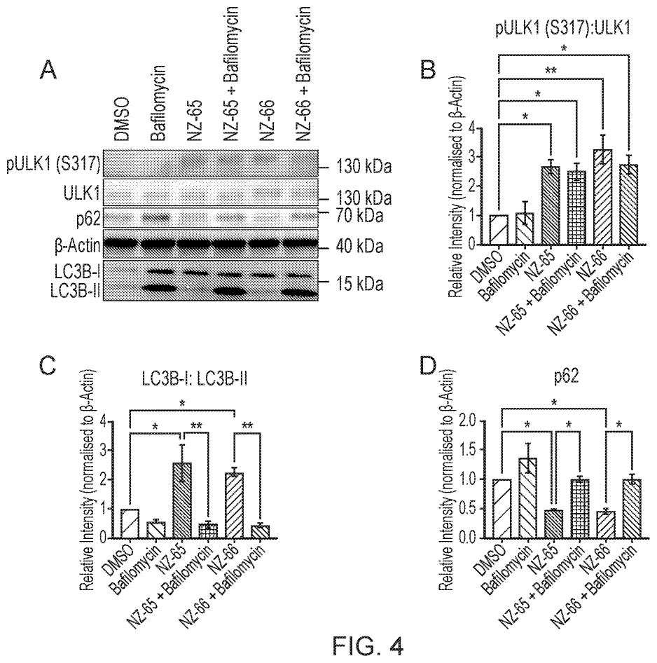

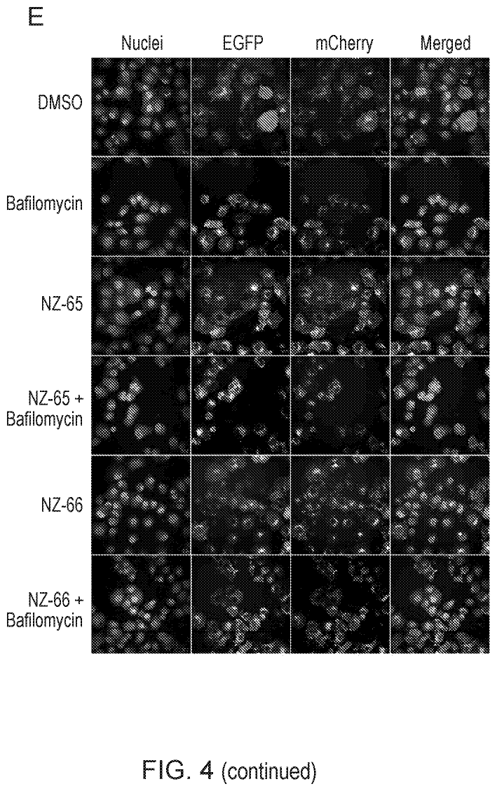

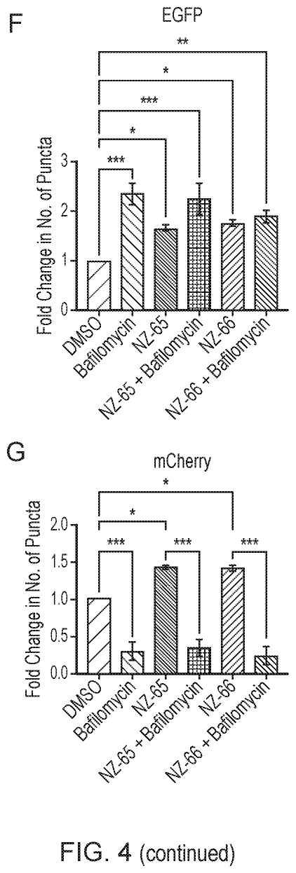

We first investigated whether modifying the ULK1 agonist by the inclusion of a linker and warhead retains agonist activity. When assessing phosphorylation of ULK1 substrates; the auto-phosphorylation of ULK1 at Ser317, we observed a strong increase in phospho-Ser317 levels, suggesting that both NZ-65 and NZ-66 retained the ability to activate ULK1 (FIG. 4A, B). In addition to this, we observed increases in the LC31: LC3II ratio coupled with mild reductions in p62/SQSTM1 (FIG. 4C, D), indicative of a mild induction of autophagy. Using the mCherry-EGFP-LC3 tandem reporter we support these findings, as we describe statistically significant changes, ranging from 1.5 and 1.3 fold increases in the number of EGFP and mCherry puncta, respectively (FIG. 4E-G). The enhanced number of mCherry puncta were reversed by co-treatment with the specific autophagy inhibitor bafilomycin A1 which blocks the vacuolar-type H+ATPase proton pump, indicating that NZ-65 and NZ-66 did not block autophagic flux.

### Example 5—Recruitment of ULK1 to Mitochondria and Induction of Mitophagy by NZ-65 and NZ-66

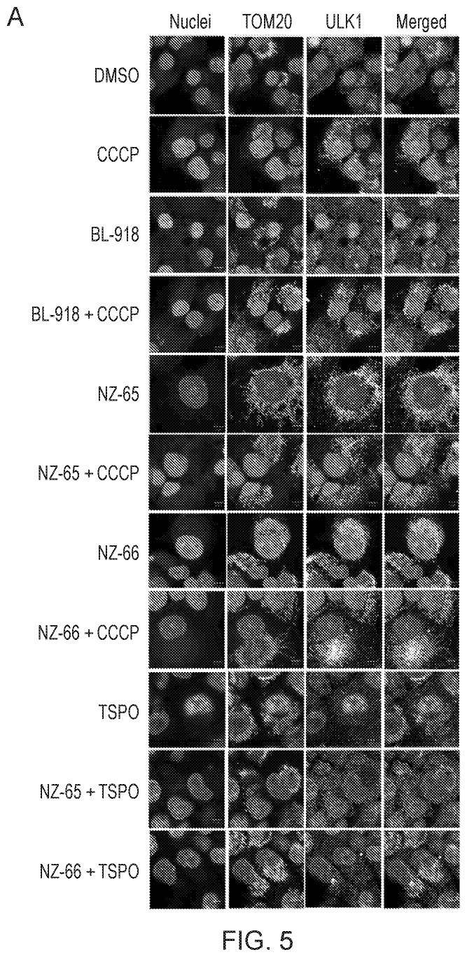

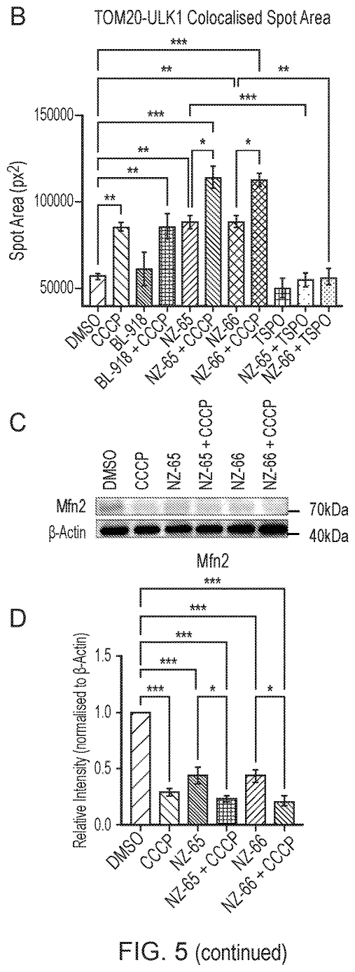

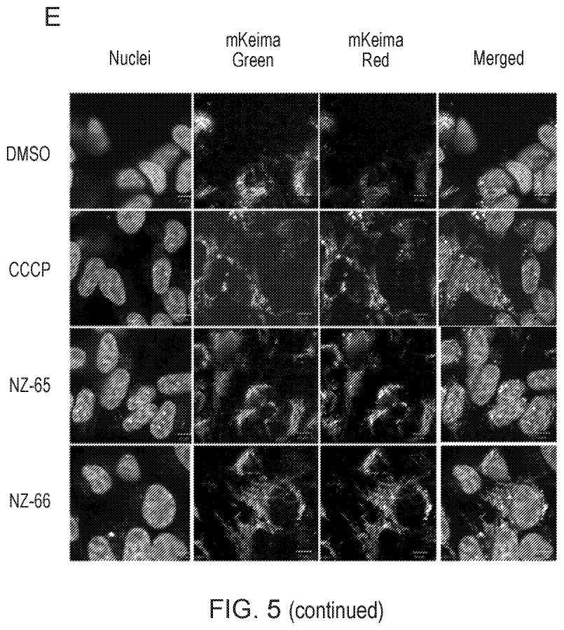

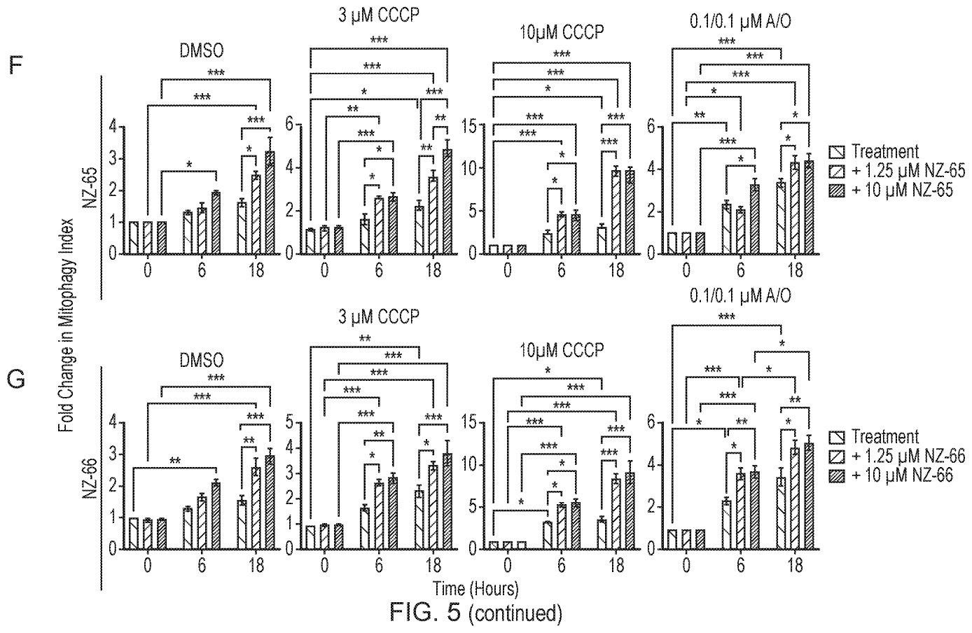

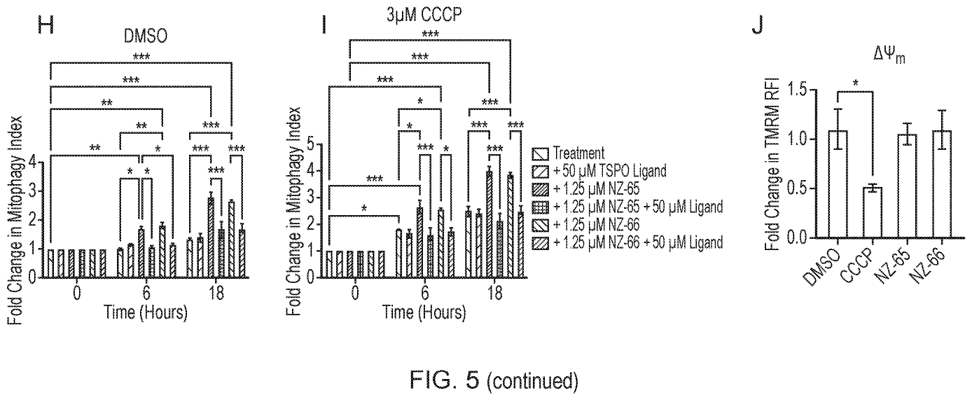

To understand the temporal kinetics of NZ-65 and NZ-66, we assessed the recruitment of ULK1 to mitochondria, using co-localisation of ULK1 and TOM20 by immunofluorescence. We observed an increased co-localisation of ULK1 and TOM20 upon treatment with NZ-65 and NZ-66 after 18 hours of treatment in PANC-1 cells (FIG. 5A, B), enhanced by CCCP co-treatment. We also showed that neither BL-918 nor the TSPO ligand alone induce co-localisation, and cotreatments of ULKTACs with excess TSPO ligand prevents ULK1 co-localisation with TOM20. This suggests that free TSPO receptor is required for ULKTAC recognition of mitochondria, and that this is facilitated only by the TSPO ligand linked to the ULK1 agonist (FIG. 5B). A similar increase in co-localisation was observed in SH-SY5Y cells. After showing the co-localisation of ULK1 to the outer mitochondrial membrane (OMM) after ULKTAC treatment, we wanted to determine whether the ULKTACs induced mitophagy. To do this, we measured changes in Mfn2 under basal conditions and co-treatment with CCCP (FIG. 5C, D). We found that the ULKTACs increase the degradation of Mfn2, enhanced upon mitochondrial insult—we found that ULKTAC-mediated degradation of Mfn2 (and hence, mitophagy) is dependent on autolysosomal activity as co-treatment with bafilomycin abolishes this activity. Similarly, we used the mito-mKeima assay to visualise these changes in mitophagy over time and observed remarkably strong increases in mitophagy upon co-treatment of NZ-65 or NZ-66 with the mitochondrial toxins CCCP and antimycin/oligomycin (FIG. 5E-G). Importantly, co-treating with excess TSPO ligand restricts enhancement of mitophagy by either NZ-65 or NZ-66 (FIG. 5H, I). When assessing the mitochondrial membrane potential with TMRM, neither NZ-65 nor NZ-66 showed any effect (FIG. 5J), hence the induction of mitophagy is not an artefact of mitochondrial membrane depolarisation. It is important to note, that these findings are absent when investigating the mitophagic potential of BL-918; instead, we observe induction of mitophagy only at higher concentrations likely due to loss of mitochondrial membrane potential as seen by a depolarisation of the outer mitochondrial membrane at higher concentrations of BL-918.

To investigate the sequence of events further we transfected PANC-1 cells with the pMRX-IP-Venus-mULK1 plasmid³¹ for 48 hours before treating cells with ULKTACs. We found that ULK1 recruitment starts approximately 3 hours after treatment and continued to be visible for 18 hours. Overall, these results suggest that NZ-65 and NZ-66 work as predicted, by recruiting ULK1 to the mitochondria, resulting in a local activation of ULK1 at the mitochondrial membrane and subsequently inducing mitophagy. A mitochondrial insult with CCCP or antimycin/oligomycin further drives the activity of both ULKTACs and enhances mitophagic activity, most likely to enable fission events to take place to enable the uptake of smaller fragments of mitochondria in the autophagosome.

### Example 6—ULK1 is Required for NZ-65- and NZ-66-Induced Mitophagy

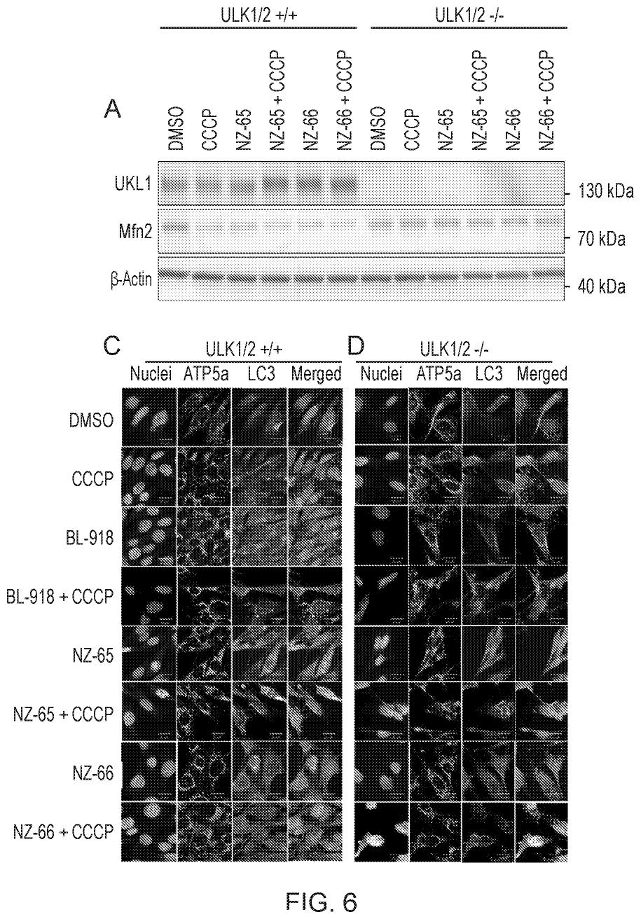

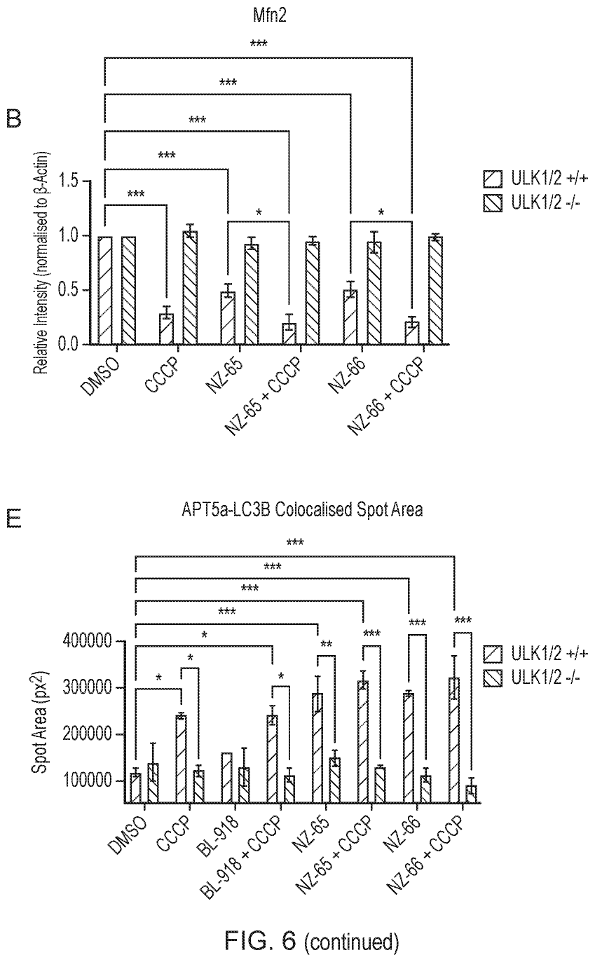

To confirm whether the activity of ULK1 is required for NZ-65 and NZ-66, we studied the recruitment of LC3 to mitochondria in ULK1/2^{-/-} mouse embryonic fibroblasts²⁷ (gift from Professor Sharon Tooze, Francis Crick Institute) and the degradation of Mfn2. In accordance with previous results, we observed a degradation of Mfn2 upon treatment with NZ-65 and NZ-66 that was further enhanced by co-treatment with CCCP; in contrast, we observed no degradation of Mfn2 in the ULK1/2^{-/-} double knockout cells upon ULKTAC treatment (FIG. 6A, B). We also observed an increase in co-localisation of the mitochondrial marker ATP5a with LC3 upon treatment with NZ-65 and NZ-66 that was absent in the ULK1/2^{-/-} cells (FIG. 6C-E), suggesting recruitment of the LC3 cargo protein to the mitochondria when MEFs are treated with NZ-65 or NZ-66, and that this is reliant on the recruitment and activation of ULK1 at the OMM. These findings were cross-validated with the recently developed dual ULK1/2 inhibitor, SBP-74556, additionally demonstrating the requirement for ULK1 for ULKTAC mediated degradation of Mfn2 and hence, mitochondria. Therefore, we conclude that NZ-65 and NZ-66 are novel ULKTAC molecules that initiate the recruitment of ULK1 to mitochondria, leading to an increase in local ULK1 activity, the formation of an autophagosome and mitophagy. Interestingly, it appears that ULK1/2 activity is required for toxin-induced mitophagy, and furthermore, NZ-65/66 enhance basal mitophagy though further enhance it in response to insult to prepare the mitochondria for uptake in an autophagosomes.

### Example 7—Testing NZ-65 and NZ-66 in Parkinson's Disease Patient-Derived Cells

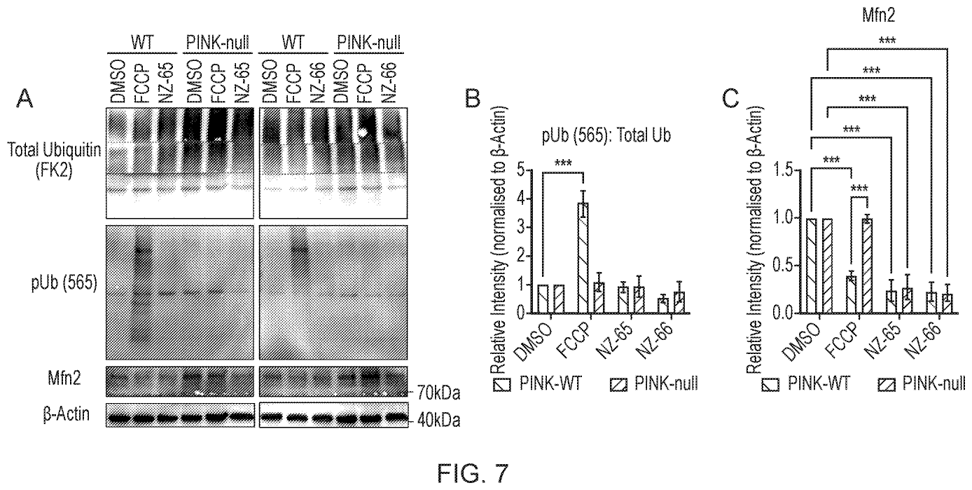

To explore the potential therapeutic benefit of the exogenous induction of mitophagy, we investigated whether the ULKTACs were able to induce mitophagy in Parkinson's disease (PD) patient-derived PINK null fibroblasts. Mutations in PINK/PRKN have been associated with PD and can predispose individuals to hereditary PD⁴⁴⁻⁴⁸. Treatment with FCCP in WT fibroblasts causes a substantial increase in p-Ub (S65) levels, which does not occur in the PINK-null cells (FIG. 7A). We find that treatment with ULKTACs does not activate phosphorylation of ubiquitin at S65, suggesting they do not activate PINK1. However we observed significant reductions in Mfn2 suggesting that the ULKTACs induce mitophagy independent of the PRKN/PINK signalling axis (FIG. 7B, C). In agreement with previous experiments, we investigated the colocalisation of ULK1 to TOM20 in both WT and PINK-null cells. In PINK-null cells, unable to signal for canonical mitophagy we do not observe colocalisation of ULK to the mitochondria validating this assay (FIG. 7D, E).

### Example 8—BRD4-Targeting Chimeric Molecule (NZ-147)

Based on the data from mitochondrial targeted NZ-65 and NZ-66, we hypothesised that using (+)-JQ1 to create a BRD4-ULKTAC would result in the simultaneous degradation of BRD4, c-MYC and MAX (c-MYC's heterodimeric interaction partner which allows it to bind DNA).

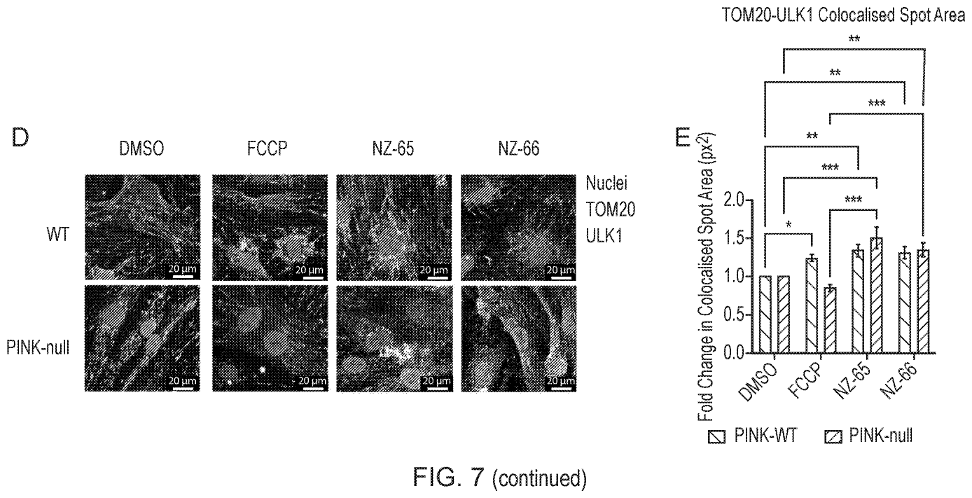

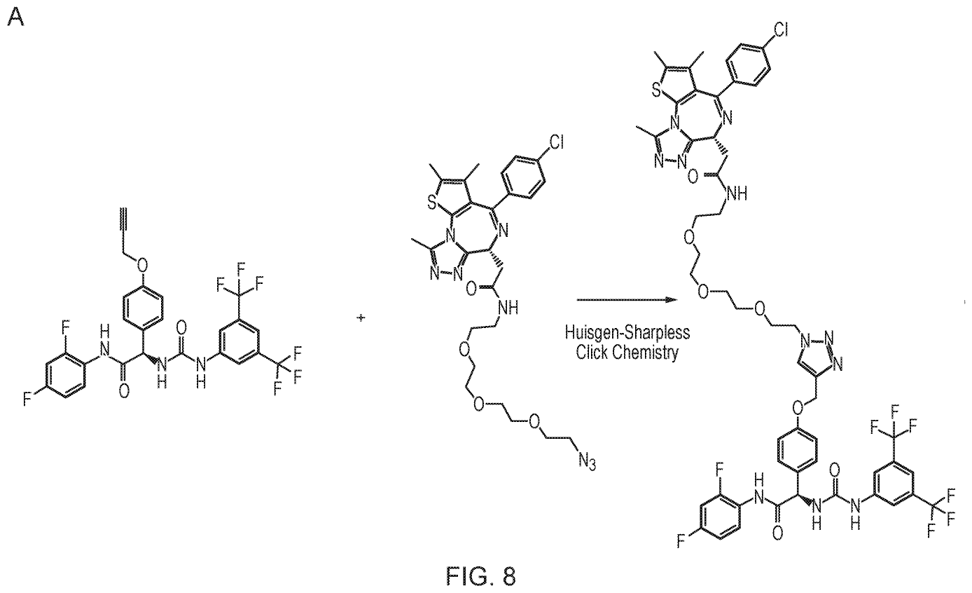

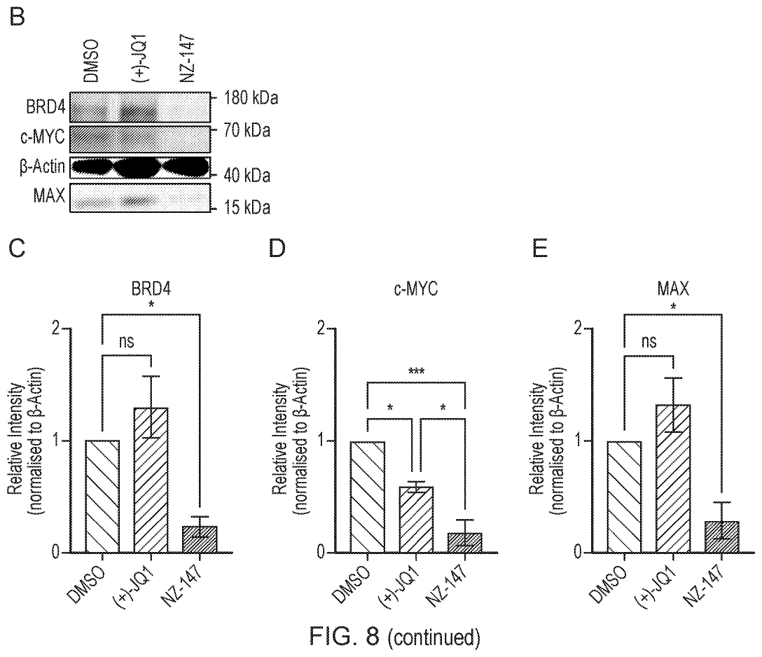

We generated a BRD4-ULKTAC (NZ-147) by conjugating (+)-JQ1 (BRD4-targeting moiety) to NZ-60 via a PEG linker (FIG. 8A). CAL51 cells (a triple negative breast cancer cell line) were treated with DMSO, (+)-JQ1 or NZ-147 at 100 nM for 72 hours then lysed and analysed by Western blot (FIG. 8B). Levels of BRD4 (FIG. 8C), c-MYC (FIG. 8D) and MAX (FIG. 8E) were reduced by treatment with NZ-147.

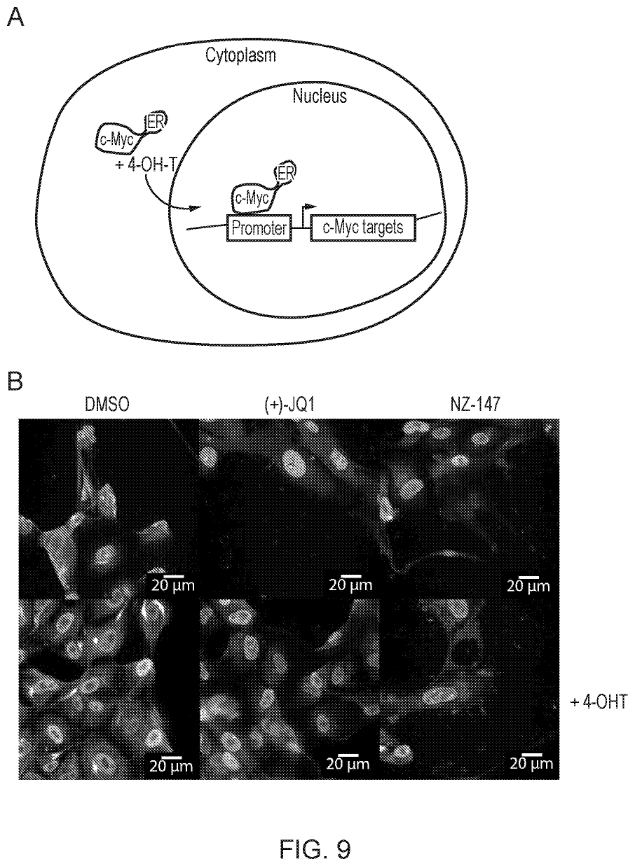

Treatment of hTERT-cMYC-ER expressing RPE1 cells with 4-hydroxytamoxifen (4-OHT) induces c-MYC-ER translocation from the cytoplasm into the nucleus, simulating oncogenic c-MYC (FIG. 9A). Immunofluorescence images of hTERT-cMYC-ER expressing RPE1 cells treated with DMSO, (+)-JQ1 or NZ-147 at 100 nM for 72 hours may show reduced nuclear c-MYC upon treatment with NZ-147 and 4-OHT (FIG. 9B).

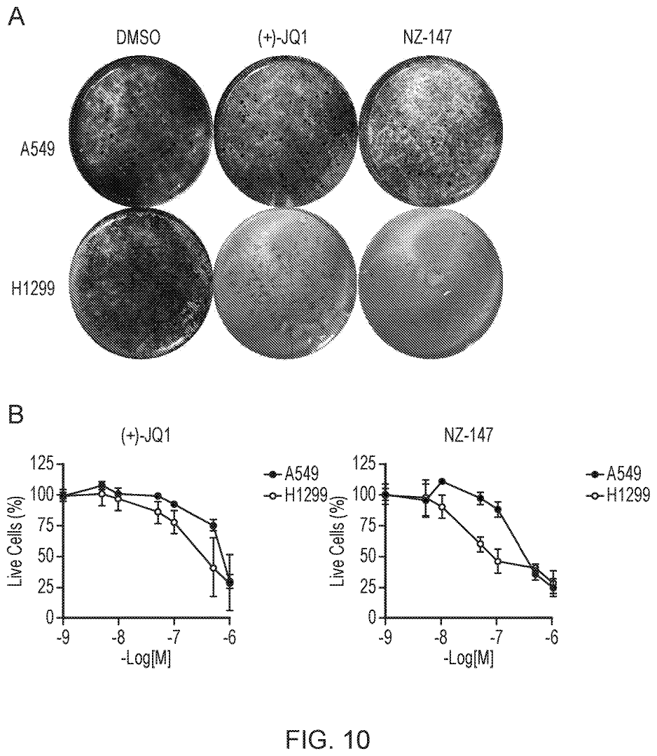

A colony formation assay was performed in A549 and H1299 cells, which were treated with DMSO, (+)-JQ1 or NZ-147 at 100 nM for 72 hours and stained with crystal violet (FIG. 10A). Fewest colonies formed in cells treated with NZ-147, suggesting reduced cell proliferation.

Cell viability assay was performed in A549 and H1299 cells, which were treated with different concentrations of (+)-JQ1 or NZ-147 at 100 nM for 72 hours (FIG. 10B). This showed a dose-dependent decrease in the percentage of live cells.

Our data suggest NZ-147 causes simultaneous degradation of BRD4, c-MYC and MAX, leading to reduced cell proliferation, exemplified further in the c-MYC-amplified cell line H1299. Using (+)-JQ1 in this manner enables a “three-hit” approach to reducing c-MYC levels and activity—at the transcriptional level by depleting BRD4, reducing c-MYC transcription, and at the protein level by actively depleting c-MYC, reducing c-MYC dependent transcription and depleting MAX, preventing c-MYC interacting with DNA.

The levels of c-MYC controlled genes (determined using RT-qPCR) in CAL51 triple negative breast cancer cell line after treatment with DMSO, 100 nM (+)-JQ1 or 100 nM NZ-147 for 72 hours is shown in FIG. 11. In particular MDM2 (FIG. 11A) and CCNE (FIG. 11B) are controlled by cMYC in oncogenic settings, PAICS (FIG. 11C) is a putative target of c-MYC, and elF4E (FIG. 11D) is a known target of c-MYC. (+)-JQ1 reduces the expression of c-MYC controlled genes; NZ-147 has a significantly more profound impact on these targets vs (+)-JQ1 at equivalent concentration and duration of treatment.

### Example 9—c-MYC-Targeting Chimeric Molecule (NZ-175)

A chimeric molecule comprising an EN4-2 derivative c-MYC-targeting moiety was conjugated to NZ-60 via a PEG-linker to synthesise NZ-175.

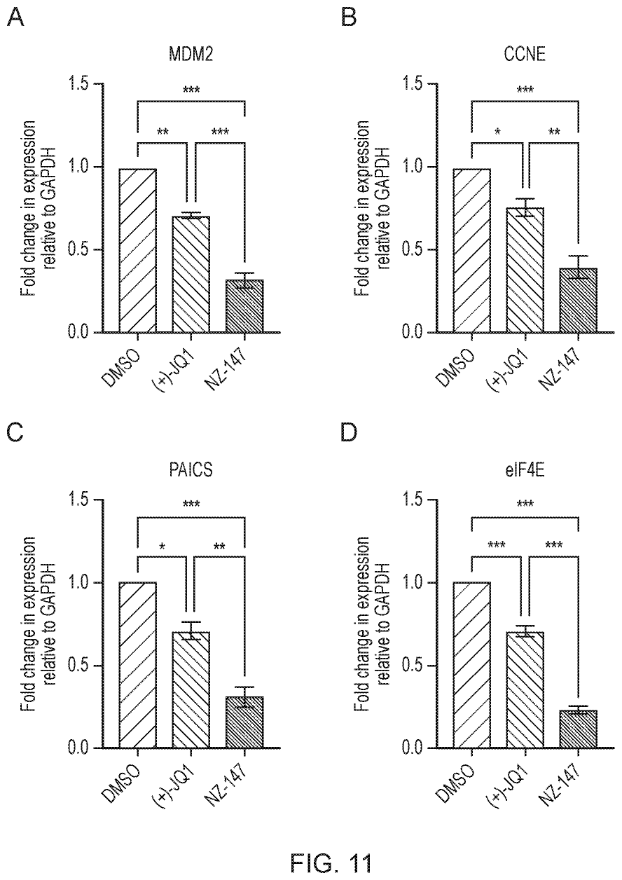

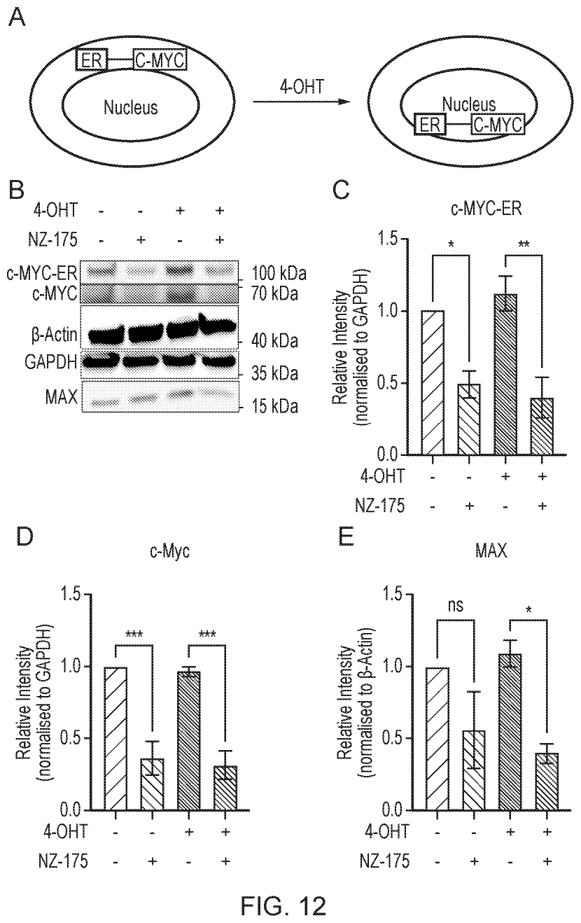

RPE1-hTERT-cMYC-ER cells were treated with 4-OHT and/or 100 nM NZ-175 for 72 h and analysed by Western blotting (FIG. 12A). The levels of c-MYC-ER (FIG. 12B), c-MYC (FIG. 12C) and MAX (FIG. 12D) were quantified. NZ-175 initiates degradation of c-MYC, and proximal degradation of MAX.

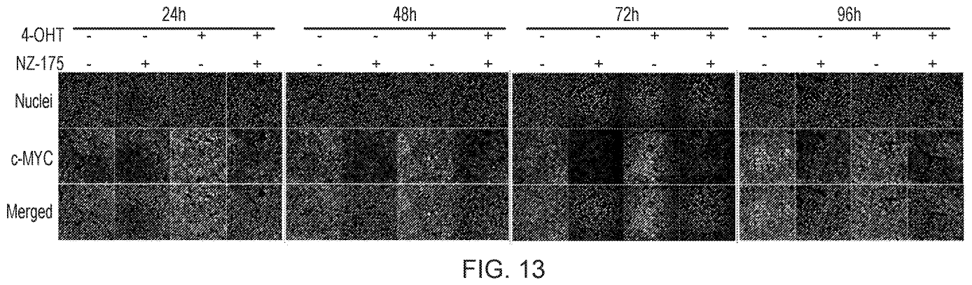

A time-course immunofluorescence of RPE1-hTERT-cMYC-ER cells treated with 4-OHT and/or 100 nM NZ-175 for 72 hours is shown in FIG. 13.

The levels of c-MYC controlled genes (determined using RT-qPCR) in RPE1-hTERT-cMYC-ER cells after treatment with 4-OHT and/or NZ-175 at 100 nM for 72 hours is shown in FIG. 14. In particular MDM2 (FIG. 14A) and CCNE (FIG. 14B) are controlled by cMYC in oncogenic settings, PAICS (FIG. 14C) is a putative target of c-MYC, elF4E (FIG. 14D) is a known target of cMYC, and MrdB (E) and ECA39 (F) are putative targets of c-MYC.

4-OHT treatment of RPE1-hTERT-cMYC-ER cells simulates oncogenic c-MYC-induced replication stress, observed by upregulation of phospho-CHK1 (S345), pRPA (S4/S8) and γH2AX.

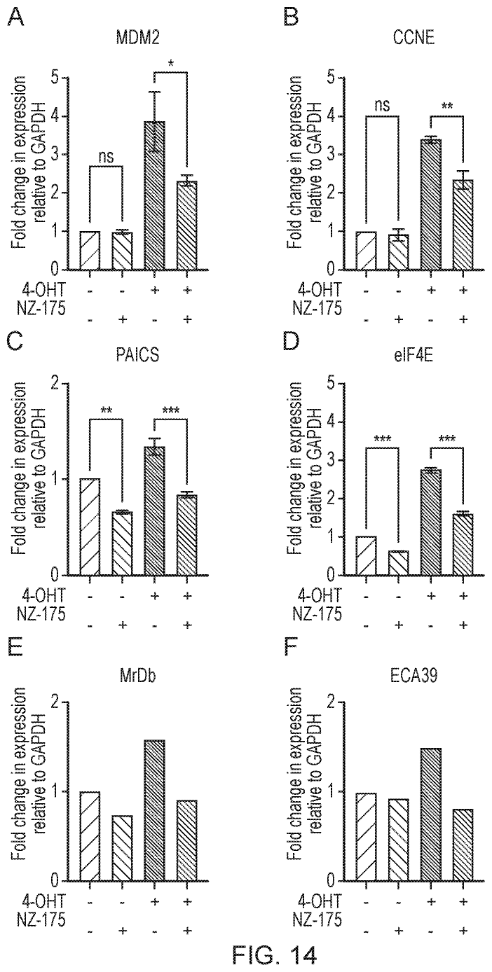

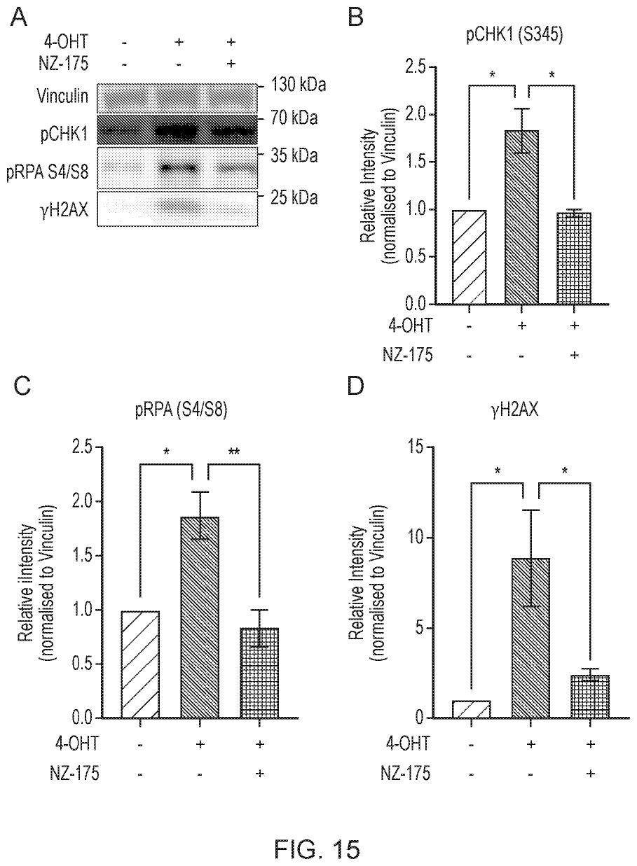

To see if NZ-175 could rescue this phenotype, RPE1-hTERT-cMYC-ER cells treated with 4-OHT and/or 100 nM NZ-175 for 72 h were analysed by Western blotting (FIG. 15A). Quantification of blot is shown for pCHK1 (S345) (FIG. 15B), pRPA (S4/S8) (FIG. 15C), and γH2AX (FIG. 15D). NZ-175 treatment rescues this phenotype.

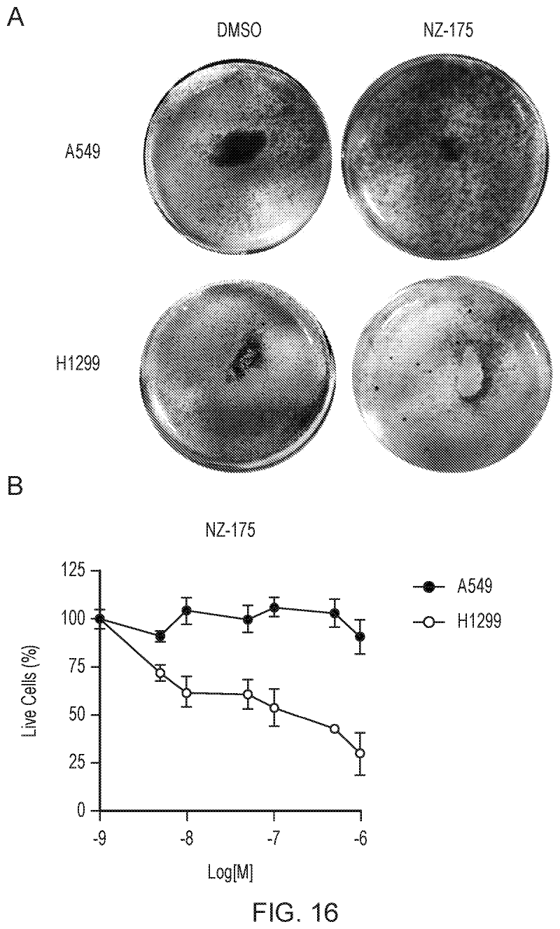

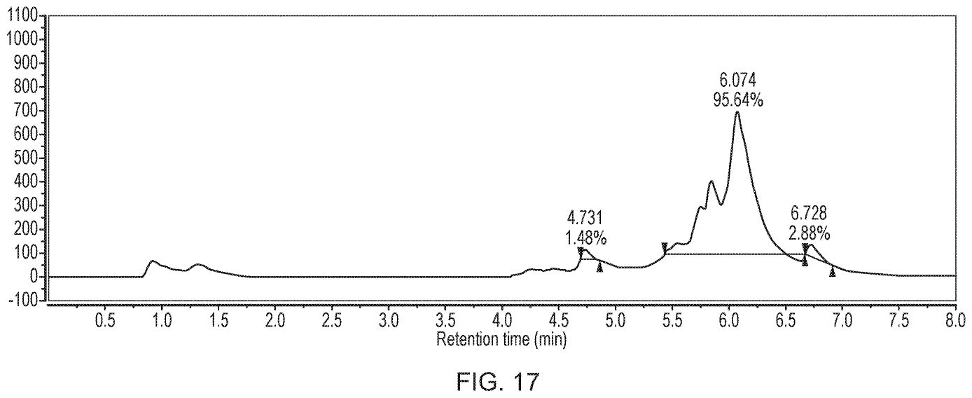

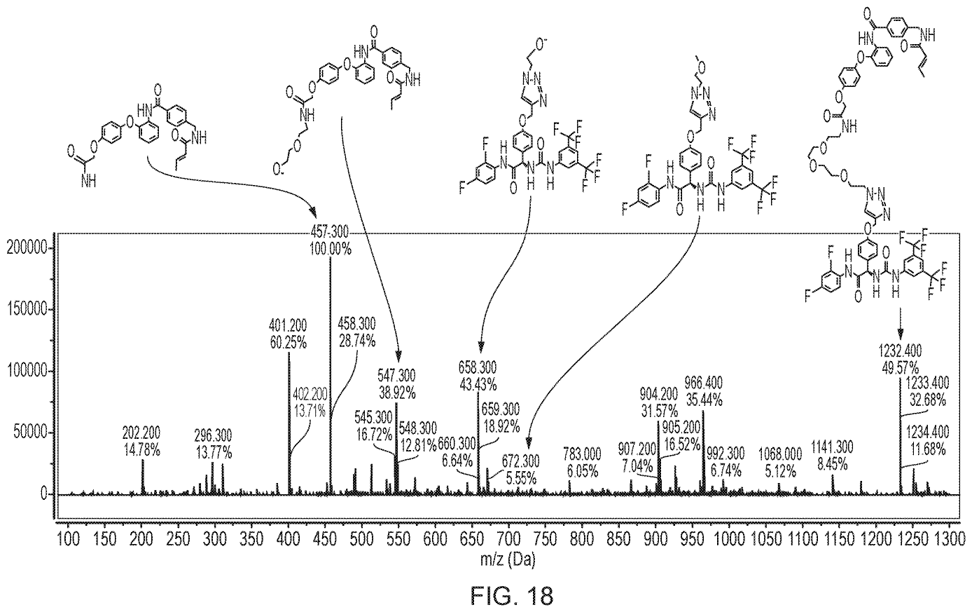

A colony formation assay was performed in A549 and H1299 cells, which were treated with DMSO or NZ-175 at 100 nM for 72 hours and stained with crystal violet (FIG. 16A). Fewer colonies formed in cells treated with NZ-175, suggesting reduced cell proliferation.

Cell viability assay was performed in A549 and H1299 cells, which were treated with different concentrations of NZ-147 at 100 nM for 72 hours (FIG. 16B). This showed a dose-dependent decrease in the percentage of live cells.

### REFERENCES

1. Aman, Y. et al. Autophagy in healthy aging and disease. Nature Aging 2021 1:8 1, 634-650 (2021).
2. Klionsky, D. J. et al. Guidelines for the use and interpretation of assays for monitoring autophagy (4th edition) 1. Autophagy 17, 1-382 (2021).
3. Klionsky, D. J. et al. Autophagy in major human diseases. EMBO J 40, (2021).
4. Li, Y. et al. A mitophagy inhibitor targeting p62 attenuates the leukemia-initiation potential of acute myeloid leukemia cells. Cancer Lett 510, 24-36 (2021).
5. Tang, F. et al. SBI0206965, a novel inhibitor of Ulk1, suppresses non-small cell lung cancer cell growth by modulating both autophagy and apoptosis pathways. Oncol Rep 37, 3449-3458 (2017).
6. Ren, H. et al. Design, Synthesis, and Characterization of an Orally Active Dual-Specific ULK1/2 Autophagy Inhibitor that Synergizes with the PARP Inhibitor Olaparib for the Treatment of Triple-Negative Breast Cancer. J Med Chem 63, 14609-14625 (2020).
7. Zhang, L. et al. Discovery of a small molecule targeting ULK1-modulated cell death of triple negative breast cancer in vitro and in vivo. Chem Sci 8, 2687-2701 (2017).
8. Ouyang, L. et al. Small-Molecule Activator of UNC-51-Like Kinase 1 (ULK1) That Induces Cytoprotective Autophagy for Parkinson's Disease Treatment. J Med Chem 61, 2776-2792 (2018).
9. Akin, D. et al. A novel ATG4B antagonist inhibits autophagy and has a negative impact on osteosarcoma tumors. Autophagy 10, 2021 (2014).
10. Agrotis, A. & Ketteler, R. On ATG4B as Drug Target for Treatment of Solid Tumours-The Knowns and the Unknowns. Cells 9, (2019).
11. Whitmarsh-Everiss, T. & Laraia, L. Small molecule probes for targeting autophagy. Nat Chem Biol 17, 653-664 (2021).
12. Samarasinghe, K. T. G. & Crews, C. M. Targeted protein degradation: A promise for undruggable proteins. Cell Chem Biol 28, 934-951 (2021).
13. Paiva, S. L. & Crews, C. M. Targeted protein degradation: elements of PROTAC design. Curr Opin Chem Biol 50, 111-119 (2019).
14. Bekes, M., Langley, D. R. & Crews, C. M. PROTAC targeted protein degraders: the past is prologue. Nature Reviews Drug Discovery 2022 21:3 21, 181-200 (2022).
15. Li, Z. et al. Allele-selective lowering of mutant HTT protein by HTT-LC3 linker compounds. Nature 575, 203-209 (2019).
16. Takahashi, D. et al. AUTACs: Cargo-Specific Degraders Using Selective Autophagy. Mol Cell 76, 797-810.e10 (2019).
17. Pei, J. et al. Developing potent LC3-targeting AUTAC tools for protein degradation with selective autophagy. Chem Commun (Camb) 57, 13194-13197 (2021).
18. Fu, Y. et al. Degradation of lipid droplets by chimeric autophagy-tethering compounds. Cell Research 2021 31:9 31, 965-979 (2021).
19. Ji, C. H. et al. The AUTOTAC chemical biology platform for targeted protein degradation via the autophagy-lysosome system. Nat Commun 13, (2022).
20. Paudel, R. R., Lu, D., Roy Chowdhury, S., Monroy, E. Y. & Wang, J. Targeted Protein Degradation via Lysosomes. Biochemistry (2022) doi: 10.1021/ACS.BIOCHEM.2C00310.
21. Xue, G. et al. Discovery of a Drug-like, Natural Product-Inspired DCAF11 Ligand Chemotype. (2023) doi: 10.26434/CHEMRXIV-2023-ZMH4F.
22. Russell, R. C. et al. ULK1 induces autophagy by phosphorylating Beclin-1 and activating VPS34 lipid kinase. Nature Cell Biology 2013 15:7 15, 741-750 (2013).
23. Osawa, T. et al. Atg2 mediates direct lipid transfer between membranes for autophagosome formation. Nat Struct Mol Biol 26, 281-288 (2019).
24. Gomez-Sanchez, R., Tooze, S. A. & Reggiori, F. Membrane supply and remodelling during autophagosome biogenesis. Curr Opin Cell Biol 71, 112-119 (2021).
25. Alemu, E. A. et al. ATG8 Family Proteins Act as Scaffolds for Assembly of the ULK Complex: SEQUENCE REQUIREMENTS FOR LC3-INTERACTING REGION (LIR) MOTIFS *. J Biol Chem 287, 39275 (2012).
26. Vargas, J. N. S. et al. Spatiotemporal Control of ULK1 Activation by NDP52 and TBK1 during Selective Autophagy. Mol Cell 74, 347-362.e6 (2019).
27. Mcalpine, F., Williamson, L. E., Tooze, S. A. & Chan, E. Y. W. Regulation of nutrient-sensitive autophagy by uncoordinated 51-like kinases 1 and 2. Autophagy 9, 361 (2013).
28. Wang, C. A Sensitive and Quantitative mKeima Assay for Mitophagy via FACS. Curr Protoc Cell Biol (2020) doi:10.1002/cpcb.99.
29. Tsefou, E. et al. Investigation of USP30 inhibition to enhance Parkin-mediated mitophagy: tools and approaches. Biochemical Journal 478, 4099-4118 (2021).
30. Agrotis, A., Pengo, N., Burden, J. J. & Ketteler, R. Redundancy of human ATG4 protease isoforms in autophagy and LC3/GABARAP processing revealed in cells. Autophagy 15, 976-997 (2019).
31. Koyama-Honda, I., Itakura, E., Fujiwara, T. K. & Mizushima, N. Temporal analysis of recruitment of mammalian ATG proteins to the autophagosome formation site. Autophagy 9, 1491-1499 (2013).
32. Daniele, S. et al. Lead Optimization of 2-Phenylindolylglyoxylyldipeptide Murine Double Minute (MDM) 2/Translocator Protein (TSPO) Dual Inhibitors for the Treatment of Gliomas. J Med Chem 59, 4526-4538 (2016).
33. Rupprecht, R. et al. Translocator protein (18 kDa) (TSPO) as a therapeutic target for neurological and psychiatric disorders. Nature Reviews Drug Discovery 2010 9:12 9, 971-988 (2010).
34. Cohen, A. S. et al. TSPO-targeted PET and Optical Probes for the Detection and Localization of Premalignant and Malignant Pancreatic Lesions. Clinical Cancer Research 26, 5914-5925 (2020).
35. Primofiore, G. et al. N,N-dialkyl-2-phenylindol-3-ylglyoxylamides. A new class of potent and selective ligands at the peripheral benzodiazepine receptor. J Med Chem 47, 1852-1855 (2004).
36. Little, D. et al. A single cell high content assay detects mitochondrial dysfunction in iPSC-derived neurons with mutations in SNCA. Sci Rep 8, (2018).
37. Creed, S. & Mckenzie, M. Measurement of Mitochondrial Membrane Potential with the Fluorescent Dye Tetramethylrhodamine Methyl Ester (TMRM). Methods Mol Biol 1928, 69-76 (2019).
38. Huisgen, R., Mloston, G. & Polborn, K. 1,3-Dipolar Activity in Cycloadditions of an Aliphatic Sulfine (,) (1). J Org Chem 61, 6570-6574 (1996).
39. Wang, Q. et al. Bioconjugation by copper (I)-catalyzed azide-alkyne [3+2] cycloaddition. J Am Chem Soc 125, 3192-3193 (2003).
40. Kolb, H. C. & Sharpless, K. B. The growing impact of click chemistry on drug discovery. Drug Discov Today 8, 1128-1137 (2003).
41. Bohmer, V. I. et al. Modular Medical Imaging Agents Based on Azide-Alkyne Huisgen Cycloadditions: Synthesis and Pre-Clinical Evaluation of 18 F-Labeled PSMA-Tracers for Prostate Cancer Imaging. Chemistry 26, 10871-10881 (2020).
42. Jaremko, Ł., Jaremko, M., Giller, K., Becker, S. & Zweckstetter, M. Structure of the mitochondrial translocator protein in complex with a diagnostic ligand. Science (1979) 343, 1363-1366 (2014).
43. Guo, Y. et al. Structure and activity of tryptophan-rich TSPO proteins. Science (1979) 347, 551-555 (2015).
44. Ge, P., Dawson, V. L. & Dawson, T. M. PINK1 and Parkin mitochondrial quality control: a source of regional vulnerability in Parkinson's disease. Molecular Neurodegeneration 2020 15:1 15, 1-18 (2020).
45. Aman, Y. et al. Enhancing mitophagy as a therapeutic approach for neurodegenerative diseases. Int Rev Neurobiol 155, 169-202 (2020).
46. Klein, C. & Westenberger, A. Genetics of Parkinson's disease. Cold Spring Harb Perspect Med 2, a008888 (2012).
47. Morais, V. A. et al. Parkinson's disease mutations in PINK1 result in decreased Complex I activity and deficient synaptic function. EMBO Mol Med 1, 99 (2009).
48. Quinn, P. M. J., Moreira, P. I., Ambrosio, A. F. & Alves, C. H. PINK1/PARKIN signalling in neurodegeneration and neuroinflammation. Acta Neuropathologica Communications 2020 8:1 8, 1-20 (2020).
49. Lu, S. L. et al. Endothelial cells are intrinsically defective in xenophagy of Streptococcus pyogenes. PLOS Pathog 13, (2017).
50. Kamber, R. A., Shoemaker, C. J. & Denic, V. Receptor-Bound Targets of Selective Autophagy Use a Scaffold Protein to Activate the Atg1 Kinase. Mol Cell 59, 372-381 (2015).
51. Torggler, R. et al. Two Independent Pathways within Selective Autophagy Converge to Activate Atg1 Kinase at the Vacuole. Mol Cell 64, 221-235 (2016).
52. Liu, W. et al. BL-918, a small-molecule activator of ULK1, induces cytoprotective autophagy for amyotrophic lateral sclerosis therapy. Acta Pharmacol Sin (2022) doi: 10.1038/S41401-022-00972-W.
53. Hijaz, B. A. & Volpicelli-Daley, L. A. Initiation and propagation of α-synuclein aggregation in the nervous system. Mol Neurodegener 15, 1-12 (2020).
54. Liu, L. et al. Mitochondrial outer-membrane protein FUNDC1 mediates hypoxiainduced mitophagy in mammalian cells. Nature Cell Biology 2012 14:2 14, 177-185 (2012).
55. Li, Y. et al. PEDF protects cardiomyocytes by promoting FUNDC1-mediated mitophagy via PEDF-R under hypoxic condition. Int J Mol Med 41, 3394-3404 (2018).
56. Zhang, W. et al. Hypoxic mitophagy regulates mitochondrial quality and platelet activation and determines severity of I/R heart injury. Elife 5, (2016).
57. Wu, W. et al. ULK1 translocates to mitochondria and phosphorylates FUNDC1 to regulate mitophagy. EMBO Rep 15, 566 (2014).
58. Kobayashi, S. et al. Mitochondrial Fission and Mitophagy Coordinately Restrict High Glucose Toxicity in Cardiomyocytes. Front Physiol 11, 1596 (2020).
59. Twig, G. & Shirihai, O. S. The Interplay Between Mitochondrial Dynamics and Mitophagy. Antioxid Redox Signal 14, 1939 (2011).
60. Shirihai, O. S., Song, M. & Dorn, G. W. How mitochondrial dynamism orchestrates mitophagy. Circ Res 116, 1835 (2015).

### NUMBERED PARAGRAPHS

The invention is described in the following numbered paragraphs:
1. A chimeric molecule for inducing degradation of a target in a cell, the chimeric molecule comprising an autophagy-inducing moiety and a targeting moiety.
2. The chimeric molecule according to paragraph 1 further comprising a linker between the autophagy-inducing moiety and the targeting moiety.
3. The chimeric molecule according to paragraph 1 or paragraph 2, wherein the chimeric molecule is represented by the formula:

A-B—C
wherein
A is the autophagy-inducing moiety,
B is an optional linker, and
C is the targeting moiety.
4. The chimeric molecule according to any one of paragraphs 1 to 3, wherein the autophagy-inducing moiety acts on Unc51 Like autophagy activating Kinase 1 (ULK1), Atg13, Atg101, FIP200, ULK2, PI3K class III, mTOR, P110 delta, FUNDC1, BNIP3, NIX, VPS34, Beclin-1 or AMPK; preferably ULK1.
5. The chimeric molecule according to paragraph 4, wherein the autophagy-inducing moiety is selected from an ULK1 binding moiety, an ULK1 agonist, an ULK2 agonist, a PI3K class Ill agonist, an mTOR inhibitor, a P110 delta antagonist, a FUNDC1 agonist, a BNIP3 agonist, a NIX agonist, a VPS34 agonist, a Beclin-1 agonist and an AMPK activator.
6. The chimeric molecule according to paragraph 5, wherein the autophagy-inducing moiety is selected from BL-918, a derivative of BL-918, LYN-1604 and a derivative of LYN-1604.
7. The chimeric molecule according to paragraph 6, wherein the ULK1 agonist is NZ-60, which has the following structure:

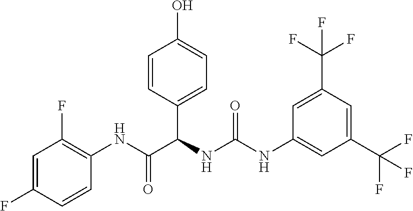 [SMILES: O=C(Nc1cc(C(F)(F)F)cc(C(F)(F)F)c1)N[C@@H](C(=O)Nc1ccc(F)cc1F)c1ccc(O)cc1]

8. The chimeric molecule according to any one of paragraphs 1 to 7, wherein the targeting moiety is selected from a mitochondria-targeting moiety, a peroxisome-targeting moiety, a soluble protein-targeting moiety, a virus-targeting moiety, a transcription factor-targeting moiety, a protein-aggregate targeting moiety, a bacterium-targeting moiety and a lipid droplet-targeting moiety.
9. The chimeric molecule according to paragraph 8, wherein the targeting moiety is a mitochondria-targeting moiety.
10. The chimeric molecule according to paragraph 9, wherein the mitochondria-targeting moiety is an outer mitochondrial membrane protein ligand, suitably a translocation protein (TSPO) ligand, a translocon of the OM (TOM) ligand, a SAM complex ligand, a voltage-dependent anion ion channel ligand or a Mitochondrial import complex (MIM) ligand.
11. The chimeric molecule according to paragraph 10, wherein the mitochondria-targeting moiety is selected from a TSPO ligand as shown in Table 1.
12. The chimeric molecule according to paragraph 11, wherein the TSPO ligand is

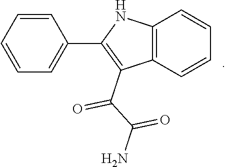 [SMILES: NC(=O)C(=O)c1c(-c2ccccc2)[nH]c2ccccc12]

13. The chimeric molecule according to any one of paragraphs 1 to 12, wherein the chimeric molecule comprises a linker between the autophagy-inducing moiety and the targeting moiety, and wherein the linker is selected from a PEG linker, an alkyl linker, an aromatic linker, a (mono) piperidine linker, a bi-piperidine linker, a (mono) piperazine and a piperidine-piperazine linker.
14. The chimeric molecule according to paragraph 13, wherein the linker is:

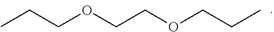

15. The chimeric molecule according to paragraph 13, wherein the linker is a 6-carbon alkyl chain.
16. The chimeric molecule according to any one of paragraphs 1 to 13, wherein the chimeric molecule is:

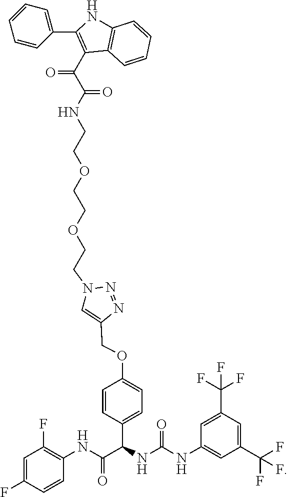 [SMILES: O=C(Nc1cc(C(F)(F)F)cc(C(F)(F)F)c1)N[C@@H](C(=O)Nc1ccc(F)cc1F)c1ccc(OCc2cn(CCOCCOCCNC(=O)C(=O)c3c(-c4ccccc4)[nH]c4ccccc34)nn2)cc1]

17. The chimeric molecule according to any one of paragraphs 1 to 13, wherein the chimeric molecule is:

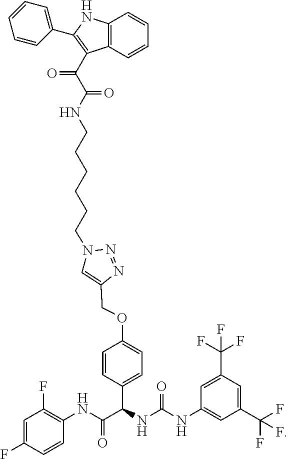 [SMILES: O=C(Nc1cc(C(F)(F)F)cc(C(F)(F)F)c1)N[C@@H](C(=O)Nc1ccc(F)cc1F)c1ccc(OCc2cn(CCCCCCNC(=O)C(=O)c3c(-c4ccccc4)[nH]c4ccccc34)nn2)cc1]

18. A pharmaceutical composition comprising the molecule according to any one of paragraphs 1 to 17 and one or more of a pharmaceutically acceptable carrier, diluent or excipient.
19. A method of treating or preventing a disease, the method comprising administering to a subject the chimeric molecule according to any one of paragraphs 1 to 17 or the pharmaceutical composition according to paragraph 18.
20. The chimeric molecule according to any one of paragraphs 1 to 17 or the pharmaceutical composition according to paragraph 18 for use in treating or preventing a disease in a subject.
21. Use of the chimeric molecule according to any one of paragraphs 1 to 17 or of the pharmaceutical composition according to paragraph 18 in the manufacture of a medicament for treating or preventing a disease in a subject.
22. The method, the chimeric molecule or pharmaceutical composition for use or the use according to any one of paragraphs 19 to 21, wherein the disease is selected from a neurodegenerative disease, cancer, viral infection, cardiovascular disease, NIBA disorders, parasitic disease, diabetes and obesity.
23. The method, the chimeric molecule or pharmaceutical composition for use or the use according to paragraph 22, wherein the disease is a neurodegenerative disease.
24. The method, the chimeric molecule or pharmaceutical composition for use or the use according to paragraph 23, wherein the neurodegenerative disease is selected from Parkinson's disease, Alzheimer's disease, amyotrophic lateral sclerosis (ALS), Friedrich's Ataxia, multiple sclerosis, Fronto-Temporal Dementia and Huntington's disease.
25. The method, the chimeric molecule or pharmaceutical composition for use or the use according to paragraph 24, wherein the neurodegenerative disease is Parkinson's disease.
26. The method, the chimeric molecule or pharmaceutical composition for use or the use according to paragraph 22, wherein the disease is cancer.
27. The method, the chimeric molecule or pharmaceutical composition for use or the use according to paragraph 26, wherein the cancer is selected from bladder cancer, gastric cancer, oesophageal cancer, breast cancer, colorectal cancer, cervical cancer, ovarian cancer, endometrial cancer, kidney cancer (renal cell), lung cancer (small cell, non-small cell and mesothelioma), brain cancer (e.g. gliomas, astrocytomas, glioblastomas), melanoma, lymphoma, small bowel cancers (duodenal and jejunal), leukemia, pancreatic cancer, hepatobiliary tumours, germ cell cancers, prostate cancer, head and neck cancers, thyroid cancer and sarcomas.
28. A chimeric molecule precursor comprising an autophagy-inducing moiety conjugated to an attachment group.
29. The chimeric molecule precursor according to paragraph 28, wherein the attachment group is selected from alkyne, amine, ester, ether, ketone, aldehyde, amide, alcohol (i.e. hydroxyl), carboxylic acid, urea, thiourea, isocyanate, isothiocynate and sulphur functional groups.
30. The chimeric molecule precursor according to paragraph 29, which is

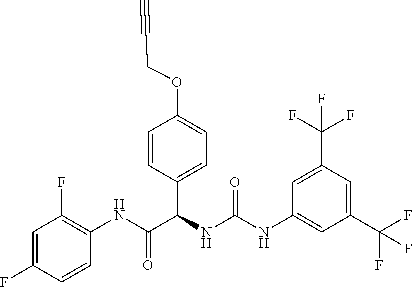 [SMILES: C#CCOc1ccc([C@@H](NC(=O)Nc2cc(C(F)(F)F)cc(C(F)(F)F)c2)C(=O)Nc2ccc(F)cc2F)cc1]

31. An in vitro or in vivo method of inducing degradation of a target in a cell, the method comprising introducing into the cell the chimeric molecule according to any one of paragraphs 1 to 17.
32. The method according to paragraph 31, wherein the target is selected from mitochondria peroxisome, protein, virus, transcription factor, protein-aggregate, bacterium and a lipid droplet.
33. The method according to paragraph 31 or paragraph 32, wherein the cell is a neuronal cell, glial cell, microglial cell, haematopoietic stem cell, red blood cell, lymphocyte, platelet, cancer cell, cardiac smooth muscle cell, skeletal muscle cell, endothelial cell, epithelial cell, hepatocyte and pancreatic beta cell.
34. An ULK1 agonist, which has the following chemical structure:

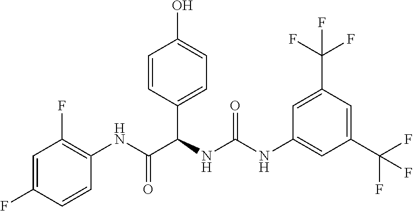 [SMILES: O=C(Nc1cc(C(F)(F)F)cc(C(F)(F)F)c1)N[C@@H](C(=O)Nc1ccc(F)cc1F)c1ccc(O)cc1]

### FURTHER NUMBERED PARAGRAPHS

The invention is also described in the following numbered paragraphs:
1. A chimeric molecule for inducing degradation of a target in a cell, the chimeric molecule comprising an autophagy-inducing moiety and a targeting moiety.
2. The chimeric molecule according to claim 1 further comprising a linker between the autophagy-inducing moiety and the targeting moiety.
3. The chimeric molecule according to claim 1 or claim 2, wherein the chimeric molecule is represented by the formula:

A-B—C
wherein
A is the autophagy-inducing moiety,
B is an optional linker, and
C is the targeting moiety.
4. The chimeric molecule according to any one of claims 1 to 3, wherein the autophagy-inducing moiety acts on Unc51 Like autophagy activating Kinase 1 (ULK1), Atg13, Atg101, FIP200, ULK2, PI3K class III, mTOR, P110 delta, FUNDC1, BNIP3, NIX, VPS34, Beclin-1 or AMPK; preferably ULK1.
5. The chimeric molecule according to claim 4, wherein the autophagy-inducing moiety is selected from an ULK1 binding moiety, an ULK1 agonist, an ULK2 agonist, a PI3K class III agonist, an mTOR inhibitor, a P110 delta antagonist, a FUNDC1 agonist, a BNIP3 agonist, a NIX agonist, a VPS34 agonist, a Beclin-1 agonist and an AMPK activator.
6. The chimeric molecule according to claim 5, wherein the autophagy-inducing moiety is selected from BL-918, a derivative of BL-918, LYN-1604 and a derivative of LYN-1604.
7. The chimeric molecule according to claim 6, wherein the ULK1 agonist is NZ-60, which has the following structure:

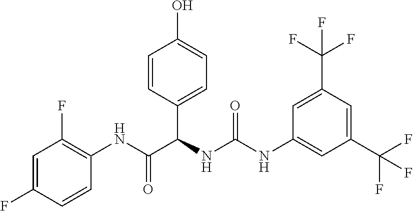 [SMILES: O=C(Nc1cc(C(F)(F)F)cc(C(F)(F)F)c1)N[C@@H](C(=O)Nc1ccc(F)cc1F)c1ccc(O)cc1]

8. The chimeric molecule according to any one of claims 1 to 7, wherein the targeting moiety is selected from a mitochondria-targeting moiety, a peroxisome-targeting moiety, a soluble protein-targeting moiety, a virus-targeting moiety, a transcription factor-targeting moiety, a protein-aggregate targeting moiety, a bacterium-targeting moiety and a lipid droplet-targeting moiety.
9. The chimeric molecule according to claim 8, wherein the targeting moiety is a mitochondria-targeting moiety, preferably wherein the mitochondria-targeting moiety is an outer mitochondrial membrane protein ligand, suitably a translocation protein (TSPO) ligand, a translocon of the OM (TOM) ligand, a SAM complex ligand, a voltage-dependent anion ion channel ligand or a Mitochondrial import complex (MIM) ligand.
10. The chimeric molecule according to claim 9, wherein the mitochondria-targeting moiety is selected from a TSPO ligand as shown in Table 1, preferably wherein the TSPO ligand is

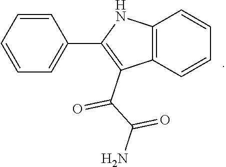 [SMILES: NC(=O)C(=O)c1c(-c2ccccc2)[nH]c2ccccc12]

11. The chimeric molecule according to any one of claims 1 to 10, wherein the chimeric molecule comprises a linker between the autophagy-inducing moiety and the targeting moiety, and wherein the linker is selected from a PEG linker, an alkyl linker, an aromatic linker, a (mono) piperidine linker, a bi-piperidine linker, a (mono) piperazine and a piperidine-piperazine linker.
12. The chimeric molecule according to claim 11, wherein the linker is:

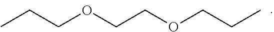

13. The chimeric molecule according to claim 11, wherein the linker is a 6-carbon alkyl chain.
14. The chimeric molecule according to any one of claims 1 to 13, wherein the chimeric molecule is:

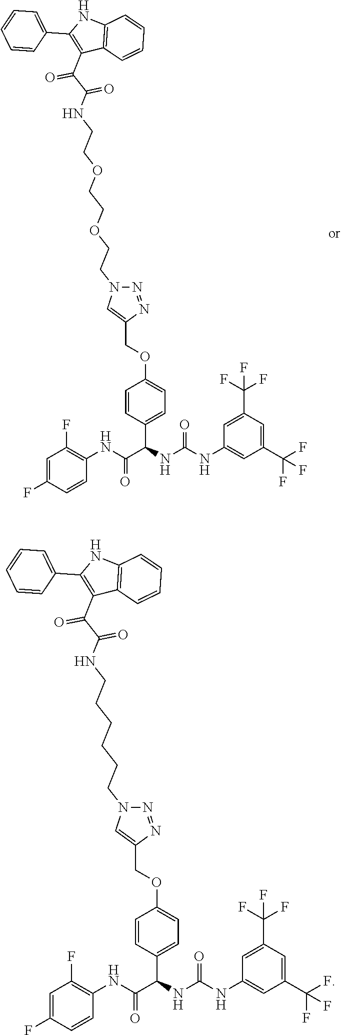 [SMILES: O=C(Nc1cc(C(F)(F)F)cc(C(F)(F)F)c1)N[C@@H](C(=O)Nc1ccc(F)cc1F)c1ccc(OCc2cn(CCOCCOCCNC(=O)C(=O)c3c(-c4ccccc4)[nH]c4ccccc34)nn2)cc1 ; O=C(Nc1cc(C(F)(F)F)cc(C(F)(F)F)c1)N[C@@H](C(=O)Nc1ccc(F)cc1F)c1ccc(OCc2cn(CCCCCCNC(=O)C(=O)c3c(-c4ccccc4)[nH]c4ccccc34)nn2)cc1]

15. A pharmaceutical composition comprising the molecule according to any one of claims 1 to 14 and one or more of a pharmaceutically acceptable carrier, diluent or excipient.
16. The chimeric molecule according to any one of claims 1 to 14 or the pharmaceutical composition according to claim 18 for use in treating or preventing a disease in a subject.
17. The chimeric molecule or pharmaceutical composition for useaccording to claim 16, wherein the disease is selected from a neurodegenerative disease, cancer, viral infection, cardiovascular disease, NIBA disorders, parasitic disease, diabetes and obesity, preferably wherein the disease is a neurodegenerative disease.
18. The chimeric molecule or pharmaceutical composition for use according to claim 17, wherein the neurodegenerative disease is selected from Parkinson's disease, Alzheimer's disease, amyotrophic lateral sclerosis (ALS), Friedrich's Ataxia, multiple sclerosis, Fronto-Temporal Dementia and Huntington's disease, preferably wherein the neurodegenerative disease is Parkinson's disease.
19. The method, the chimeric molecule or pharmaceutical composition for use or the use according to claim 16, wherein the disease is cancer, preferably wherein the cancer is selected from bladder cancer, gastric cancer, oesophageal cancer, breast cancer, colorectal cancer, cervical cancer, ovarian cancer, endometrial cancer, kidney cancer (renal cell), lung cancer (small cell, non-small cell and mesothelioma), brain cancer (e.g. gliomas, astrocytomas, glioblastomas), melanoma, lymphoma, small bowel cancers (duodenal and jejunal), leukemia, pancreatic cancer, hepatobiliary tumours, germ cell cancers, prostate cancer, head and neck cancers, thyroid cancer and sarcomas.
20. A chimeric molecule precursor comprising an autophagy-inducing moiety conjugated to an attachment group.
21. The chimeric molecule precursor according to claim 20, wherein the attachment group is selected from alkyne, amine, ester, ether, ketone, aldehyde, amide, alcohol (i.e. hydroxyl), carboxylic acid, urea, thiourea, isocyanate, isothiocynate and sulphur functional groups.
22. The chimeric molecule precursor according to claim 21, which is

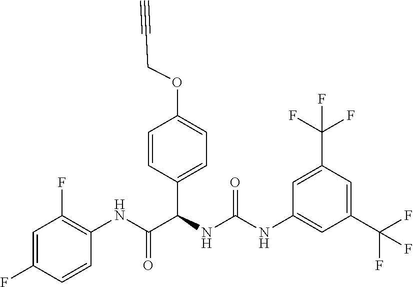 [SMILES: C#CCOc1ccc([C@@H](NC(=O)Nc2cc(C(F)(F)F)cc(C(F)(F)F)c2)C(=O)Nc2ccc(F)cc2F)cc1]

23. An in vitro or in vivo method of inducing degradation of a target in a cell, the method comprising introducing into the cell the chimeric molecule according to any one of claims 1 to 14.
24. The method according to claim 23, wherein the cell is a neuronal cell, glial cell, microglial cell, haematopoietic stem cell, red blood cell, lymphocyte, platelet, cancer cell, cardiac smooth muscle cell, skeletal muscle cell, endothelial cell, epithelial cell, hepatocyte and pancreatic beta cell.
25. An ULK1 agonist, which has the following chemical structure:

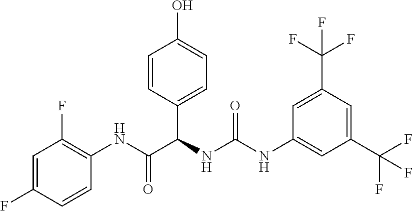 [SMILES: O=C(Nc1cc(C(F)(F)F)cc(C(F)(F)F)c1)N[C@@H](C(=O)Nc1ccc(F)cc1F)c1ccc(O)cc1]

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in virology, molecular biology or related fields are intended to be within the scope of the following claims.

1. A chimeric molecule for inducing degradation of a target in a cell, the chimeric molecule comprising an autophagy-inducing moiety and a targeting moiety.

2. The chimeric molecule according to claim 1 further comprising a linker between the autophagy-inducing moiety and the targeting moiety.

3. The chimeric molecule according to claim 1 or claim 2, wherein the chimeric molecule is represented by the formula:
A-B—C
wherein
A is the autophagy-inducing moiety,
B is an optional linker, and
C is the targeting moiety.

4. The chimeric molecule according to any one of claims 1 to 3, wherein the autophagy-inducing moiety acts on Unc51 Like autophagy activating Kinase 1 (ULK1), Atg13, Atg101, FIP200, ULK2, PI3K class III, mTOR, P110 delta, FUNDC1, BNIP3, NIX, VPS34, Beclin-1 or AMPK; preferably ULK1.

5. The chimeric molecule according to claim 4, wherein the autophagy-inducing moiety is selected from an ULK1 binding moiety, an ULK1 agonist, an ULK2 agonist, a PI3K class III agonist, an mTOR inhibitor, a P110 delta antagonist, a FUNDC1 agonist, a BNIP3 agonist, a NIX agonist, a VPS34 agonist, a Beclin-1 agonist and an AMPK activator.

6. The chimeric molecule according to claim 5, wherein the autophagy-inducing moiety is selected from BL-918, a derivative of BL-918, LYN-1604 and a derivative of LYN-1604.

7. The chimeric molecule according to claim 6, wherein the ULK1 agonist is NZ-60, which has the following structure:

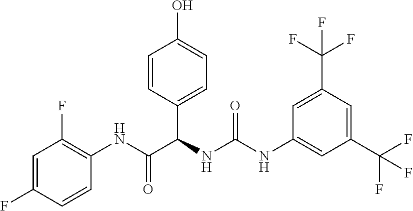 [SMILES: O=C(Nc1cc(C(F)(F)F)cc(C(F)(F)F)c1)N[C@@H](C(=O)Nc1ccc(F)cc1F)c1ccc(O)cc1]

8. The chimeric molecule according to any one of claims 1 to 7, wherein the chimeric molecule comprises a linker between the autophagy-inducing moiety and the targeting moiety, and wherein the linker is selected from a PEG linker, an alkyl linker, an aromatic linker, a (mono) piperidine linker, a bi-piperidine linker, a (mono) piperazine and a piperidine-piperazine linker.

9. The chimeric molecule according to claim 8, wherein the linker comprises or consists of: 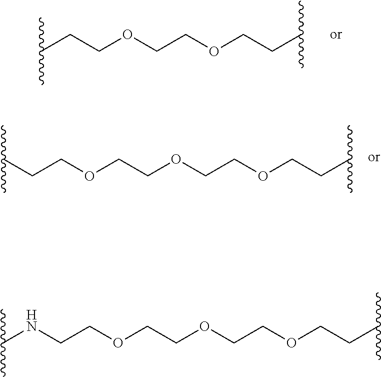 [SMILES: *CCOCCOCC* ; *CCOCCOCCOCC* ; *CCOCCOCCOCCN*]
or
a 6-carbon alkyl chain.

10. The chimeric molecule according to any one of claims 1 to 9, wherein the targeting moiety is selected from a mitochondria-targeting moiety, a peroxisome-targeting moiety, a soluble protein-targeting moiety, a virus-targeting moiety, a transcription factor-targeting moiety, a protein-aggregate targeting moiety, a bacterium-targeting moiety and a lipid droplet-targeting moiety.

11. The chimeric molecule according to any one of claims 1 to 10, wherein the targeting moiety is a BRD4-targeting moiety.

12. The chimeric molecule according to claim 11, wherein the BRD4-targeting moiety is (+)-JQ1:

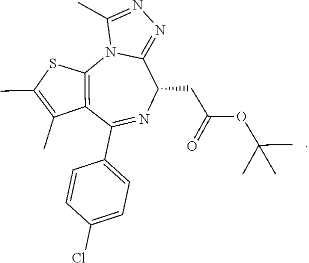 [SMILES: Cc1sc2c(c1C)C(c1ccc(Cl)cc1)=N[C@@H](CC(=O)OC(C)(C)C)c1nnc(C)n1-2]

13. The chimeric molecule according to any one of claims 1 to 12, wherein the chimeric molecule is:

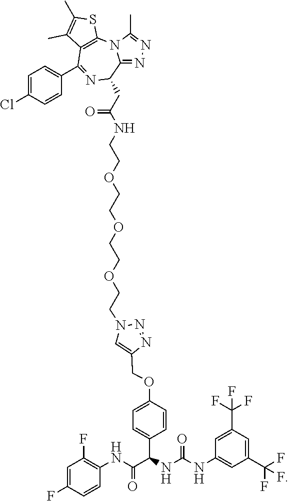 [SMILES: Cc1sc2c(c1C)C(c1ccc(Cl)cc1)=N[C@@H](CC(=O)NCCOCCOCCOCCn1cc(COc3ccc([C@@H](NC(=O)Nc4cc(C(F)(F)F)cc(C(F)(F)F)c4)C(=O)Nc4ccc(F)cc4F)cc3)nn1)c1nnc(C)n1-2]

14. The chimeric molecule according to any one of claims 1 to 10, wherein the targeting moiety is a c-MYC targeting moiety.

15. The chimeric molecule according to claim 14, wherein the c-MYC targeting moiety is selected from the molecules shown in Table 6.

16. The chimeric molecule according to claim 14, wherein the c-MYC-targeting moiety is the following derivative of EN4-2:

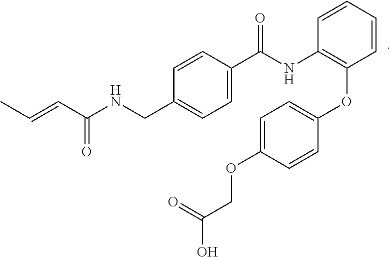 [SMILES: C/C=C/C(=O)NCc1ccc(C(=O)Nc2ccccc2Oc2ccc(OCC(=O)O)cc2)cc1]

17. The chimeric molecule according to claim 16, wherein the chimeric molecule is:

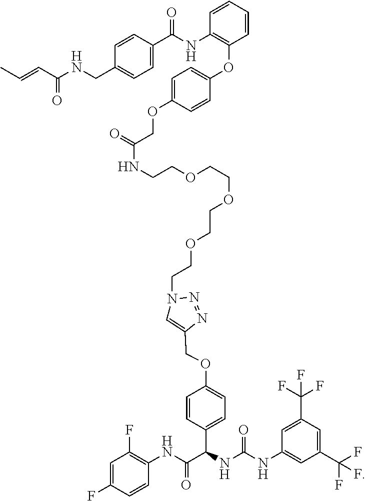 [SMILES: C/C=C/C(=O)NCc1ccc(C(=O)Nc2ccccc2Oc2ccc(OCC(=O)NCCOCCOCCOCCn3cc(COc4ccc([C@@H](NC(=O)Nc5cc(C(F)(F)F)cc(C(F)(F)F)c5)C(=O)Nc5ccc(F)cc5F)cc4)nn3)cc2)cc1]

18. The chimeric molecule according to claim 10, wherein the targeting moiety is a mitochondria-targeting moiety, preferably wherein the mitochondria-targeting moiety is an outer mitochondrial membrane protein ligand, suitably a translocation protein (TSPO) ligand, a translocon of the OM (TOM) ligand, a SAM complex ligand, a voltage-dependent anion ion channel ligand or a Mitochondrial import complex (MIM) ligand.

19. The chimeric molecule according to claim 18, wherein the mitochondria-targeting moiety is selected from a TSPO ligand as shown in Table 1, preferably wherein the TSPO ligand is

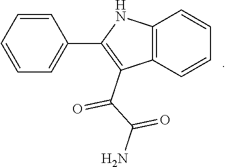 [SMILES: NC(=O)C(=O)c1c(-c2ccccc2)[nH]c2ccccc12]

20. The chimeric molecule according to claim 19, wherein the chimeric molecule is:

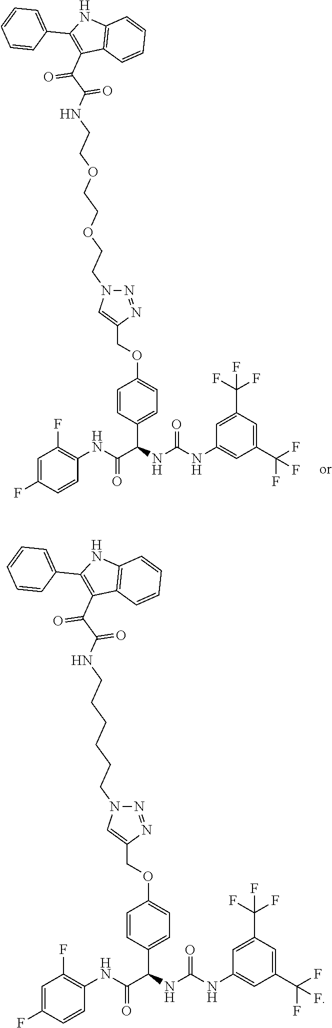 [SMILES: O=C(Nc1cc(C(F)(F)F)cc(C(F)(F)F)c1)N[C@@H](C(=O)Nc1ccc(F)cc1F)c1ccc(OCc2cn(CCOCCOCCNC(=O)C(=O)c3c(-c4ccccc4)[nH]c4ccccc34)nn2)cc1 ; O=C(Nc1cc(C(F)(F)F)cc(C(F)(F)F)c1)N[C@@H](C(=O)Nc1ccc(F)cc1F)c1ccc(OCc2cn(CCCCCCNC(=O)C(=O)c3c(-c4ccccc4)[nH]c4ccccc34)nn2)cc1]

21. A pharmaceutical composition comprising the molecule according to any one of claims 1 to 20 and one or more of a pharmaceutically acceptable carrier, diluent or excipient.

22. The chimeric molecule according to any one of claims 1 to 20 or the pharmaceutical composition according to claim 21 for use in treating or preventing a disease in a subject.

23. The chimeric molecule or pharmaceutical composition for use according to claim 22, wherein the disease is selected from cancer, a neurodegenerative disease, viral infection, cardiovascular disease, NIBA disorders, parasitic disease, diabetes and obesity, preferably wherein the disease is a neurodegenerative disease.

24. The chimeric molecule or pharmaceutical composition for use according to claim 22, wherein the disease is cancer, preferably wherein the cancer is selected from bladder cancer, gastric cancer, oesophageal cancer, breast cancer, colorectal cancer, cervical cancer, ovarian cancer, endometrial cancer, kidney cancer (renal cell), lung cancer (small cell, non-small cell and mesothelioma), brain cancer (e.g. gliomas, astrocytomas, glioblastomas), melanoma, lymphoma, small bowel cancers (duodenal and jejunal), leukemia, pancreatic cancer, hepatobiliary tumours, germ cell cancers, prostate cancer, head and neck cancers, thyroid cancer and sarcomas.

25. The chimeric molecule or pharmaceutical composition for use according to claim 23 wherein the disease is any of the following cancers: endometrial, non-small cell lung (including adenocarcinoma and squamous cell lung carcinoma), breast (including luminal A, luminal B, HER2+ and TNBC breast cancer), ovarian, oesophageal, liver, prostate, head-and-neck squamous cell carcinoma (HNSCC), liver, pancreatic, colorectal, neuroblastoma, B-cell lymphomas, renal clear cell carcinoma, adrenal cell carcinoma, medulloblastoma, and haematological cancers (especially T-ALL, B-ALL, AML, myeloma, and lymphoma, especially Burkitt's lymphoma (MYC-IgH+) and MYC positive DLBCL).

26. The chimeric molecule or pharmaceutical composition for use according to claim 23, wherein the neurodegenerative disease is selected from Parkinson's disease, Alzheimer's disease, amyotrophic lateral sclerosis (ALS), Friedrich's Ataxia, multiple sclerosis, Fronto-Temporal Dementia and Huntington's disease, preferably wherein the neurodegenerative disease is Parkinson's disease.

27. A chimeric molecule precursor comprising an autophagy-inducing moiety conjugated to an attachment group.

28. The chimeric molecule precursor according to claim 27, wherein the attachment group is selected from alkyne, amine, ester, ether, ketone, aldehyde, amide, alcohol (i.e. hydroxyl), carboxylic acid, urea, thiourea, isocyanate, isothiocynate and sulphur functional groups.

29. The chimeric molecule precursor according to claim 28, which is

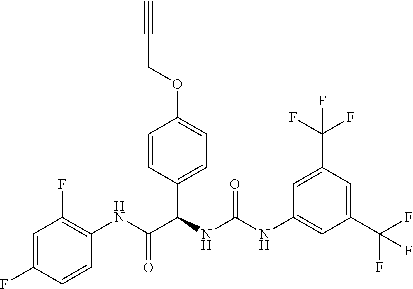 [SMILES: C#CCOc1ccc([C@@H](NC(=O)Nc2cc(C(F)(F)F)cc(C(F)(F)F)c2)C(=O)Nc2ccc(F)cc2F)cc1]

30. An in vitro or in vivo method of inducing degradation of a target in a cell, the method comprising introducing into the cell the chimeric molecule according to any one of claims 1 to 20.

31. The method according to claim 30, wherein the cell is a neuronal cell, glial cell, microglial cell, haematopoietic stem cell, red blood cell, lymphocyte, platelet, cancer cell, cardiac smooth muscle cell, skeletal muscle cell, endothelial cell, epithelial cell, hepatocyte or pancreatic beta cell.

32. An ULK1 agonist, which has the following chemical structure: 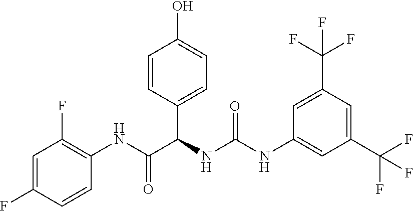 [SMILES: O=C(Nc1cc(C(F)(F)F)cc(C(F)(F)F)c1)N[C@@H](C(=O)Nc1ccc(F)cc1F)c1ccc(O)cc1]
